(19) United States
(12) Patent Application Publication
(10) Pub. No.: US 2025/0136559 A1
(43) Pub. Date: May 1, 2025

(54) SMO MODULATOR COMPOUNDS

(71) Applicant: OMASS THERAPEUTICS LIMITED, Oxford (GB)

(72) Inventors: Giles Albert Brown, Oxford (GB); Laia Malet Sanz, Oxford (GB); Benjamin Tehan, Oxford (GB); Omprakash Bande, Bangalore (IN)

(21) Appl. No.: 18/834,797

(22) PCT Filed: Feb. 1, 2023
(86) PCT No.: PCT/GB2023/050218

§ 371 Date: Jul. 31, 2024

(30) Foreign Application Priority Data
Feb. 1, 2022 (GB) 2201283.5

(51) Int. Cl. C07D 249/08; A61K 31/541; C07D 231/12; C07D 261/08; C07D 263/32; C07D 271/06; C07D 271/113; C07D 401/04; C07D 401/06; C07D 403/04; C07D 405/06; C07D 409/04; C07D 413/04; C07D 417/12

(52) U.S. Cl. CPC C07D 249/08; A61K 31/541; C07D 231/12; C07D 261/08; C07D 263/32; C07D 271/06; C07D 271/113; C07D 401/04; C07D 401/06; C07D 403/04; C07D 405/06; C07D 409/04; C07D 413/04; C07D 417/12

(57) ABSTRACT

The disclosures herein relate to novel compounds of Formula (1a) or a salt thereof, wherein X¹, X², X³, X⁴, Y¹, Y², Y³, Y⁴, Z¹, Z², Z³, Z⁴, W, R¹, R⁴, R⁵, R⁶ and R⁷ are defined herein, and their use in treating, preventing, ameliorating, controlling or reducing the risk of disorders associated with SMO receptors.

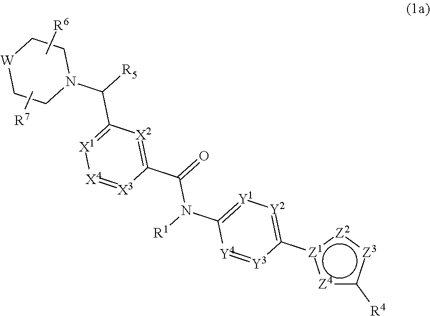

This application relates to novel compounds and their use as Smoothened (SMO) receptor antagonists. Compounds described herein may be useful in the treatment or prevention of diseases in which SMO receptors are involved or in the treatment or prevention of hedgehog (Hh) signalling pathway-associated diseases, such as cancer. This application is also directed to pharmaceutical compositions comprising these compounds and the manufacture and use of these compounds and compositions in the prevention or treatment of such diseases in which SMO receptors are involved.

### BACKGROUND OF THE INVENTION

The Hedgehog (Hh) pathway is a highly conserved signalling pathway, exerting pivotal roles in cell proliferation and patterning during embryogenesis in metazoa. During adulthood, the Hh pathway is silenced in most cell types and tissues, however it continues to control homeostasis and tissue repair in stem cells and the skin. Abnormal activation of the Hh pathway can lead to a number of pathological conditions, including fibrosis and cancer.

Regulation of the Hh pathway is achieved through a complex cascade of biochemical events involving multiple components. A central role in Hh signal transduction is played by the Smoothened (SMO) receptor, a Class F 7-transmembrane G protein-coupled receptor (GPCR) that functions to transmit extracellular signals across the cell membrane and activate the Hh signalling pathway within the cell. SMO receptor activity is controlled by the 12-transmembrane domain receptor Patched 1 (PTCH1), which catalytically represses SMO in the absence of its endogenous ligand Sonic Hedgehog (SHh). Sonic hedgehog is the best understood and studied protein of a total of three hedgehog ligands (Sonic, Desert and Indian), which are synthesised as precursor proteins that undergo autocatalytic processing to yield active N-terminal signalling domains. Upon SHH binding to PTCH1, SMO inhibition is lifted, thereby permitting the activation of SMO and the Hh signalling pathway. The mechanism of SMO inhibition by PTCH1 is not yet fully understood, however it is thought that PTCH1 uses its cholesterol transporter function to regulate the accessibility of cholesterol and oxysterols to the SMO receptor. It has been reported that cholesterol activates SMO, and inhibition of PTCH1 would increase the accessibility of cholesterol and thereby its concentration around SMO, allowing receptor activation.

As the SMO receptor is activated, it translocates and accumulates in primary cilia (and is phosphorylated by PKA, CK1/2 and GRK2 to induce conformational changes. The exact sequence of molecular downstream events in the Hh signalling pathway following SMO receptor activation is still being elucidated, however a key step in the activation of the Hh pathway via SMO involves in the dissociation of glioma-associated (GLI) transcription factors from the suppressor-of-fused protein (Sufu) and, to a lesser extent, kinesin-family protein (Kif7). The accumulation of GLI transcription factors represent the terminal effectors of the Hh signalling cascade, with GLI proteins being differentially phosphorylated and processed into transcriptional activators (GLI1, GLI2) or repressors (GLI3). Upon their translocation into the nucleus, they regulate the expression of target genes, including PTCH1, cyclin, FOX and GLI genes.

In humans, genetic mutations that result in constitutively active Hh pathway signalling contribute to the development of various malignancies including pancreatic, breast, colon, ovarian and small-cell lung carcinomas, with mutations in this pathway most consistently having been observed in medulloblastomas and basal cell carcinomas (BCCs).

BCCs represent the most common type of skin cancer, and although the majority of BCCs are characterised by low metastatic potential, advanced BCCs can lead to enhanced metastasis and mortality. BCC development has been linked to environmental factors, such as age or increased exposure to radiation, as well as an underlying genetic predisposition. BCC tumours are highly heterogeneous, displaying a plethora of histological subtypes, each carrying multiple mutations; the average mutation cargo in a BCC tumour has been estimated to be as high as 2,000 somatic coding mutations per lesion. The most prevalent genetic changes in BCC patients are inactivating mutations to Hh suppressors (most commonly PTCH1) and/or gain-of-function mutations to pathway activators (such as SMO or SHh), both resulting in constitutively active Hh signalling and high expression levels of GLI genes.

Targeting SMO with small-molecule antagonists has been a particularly attractive screening strategy for the development of BCC therapeutics as such therapies are also be beneficial for targeting tumours harbouring inactivating PTCH1 mutations. Screening efforts have resulted in the discovery and clinical approval of two small molecule inhibitors, namely vismodegib (GDC-0449) and sonidegib (LDE-225) approved by the FDA in 2012 and 2015, respectively, for the treatment of locally advanced or metastatic BCCs. Glasdegib (PF-0444991) was approved by the FDA in 2018 and is currently being used for the treatment of acute myeloid leukemia. Although all three drugs offer clinical benefits, patient response rates do not typically surpass 50%, and the occurrence of multiple adverse effects such as muscle spasms, alopecia, dysgeusia and weight loss, in combination with fast-induced acquired resistance and tumour re-growth ultimately leads to high discontinuation rates, highlighting a clear need for improved therapeutics for these patients.

The SMO receptor has a large N-terminal cysteine-rich domain in addition to the classical 7-transmembrane domain (TMD) characteristic of GPCRs. Both, the CRD and a pocket within the TMD have been identified as binding site for both cholesterol and oxysterol which are also able to induce activation of the Hh signalling pathway increasing GLI1 transcription levels.

Recent SMO structural studies have provided some insight into potential receptor conformations in the absence and presence of activating and inhibiting ligands. As such, Vismodegib has been reported to inhibit SMO through a conformational change that prevents cholesterol from binding and has been shown to bind at the extracellular end of the TM bundle, whilst also making contacts with the ligand binding pocket of the TMD.

The aitiology behind primary and secondary (acquired) resistance to available SMO antagonists is not yet fully elucidated. A number of studies describe that the emergence of genetic alterations to downstream effectors of the pathway (e.g. SUFU, GLI2, CyclinD1) can confer primary resistance to SMO inhibitors. However, SMO is the most recurrently mutated gene found in resistant BCCs and medulloblastomas. Reported mechanistic insights show that the SMO mutant variants either directly impair drug binding or lead to a constitutively active SMO receptor, enabling the carrier cells to grow faster thereby displacing neighbouring cells that express wild-type SMO receptors. Over the years, numerous SMO mutations have been identified in the clinic, such as mutations affecting residues that are located either directly within the ligand binding pocket of the transmembrane domain directly affecting ligand binding properties or TMD mutations that cause conformational changes affecting the ligand binding pocket and ligand binding indirectly. These mutations for example include D473, W28I, Q477 and I408, and usually appear in post-treatment specimens of Vismodegib-resistant BCCs patients. Oncogenic mutations affecting other crucial residues of the SMO transmembrane region outside the ligand binding pocket, such as T241M, W535L, L421, A459V, V321M, F460L and S533N have been found both in both untreated and vismodegib-resistant BCCs, as they confer constitutive activity in the absence of SHh ligand. Mutations S533N, L421F and W535L have been classified as activating mutations, characterised by very high baseline activity levels that evade SMO antagonist treatment. On the other hand, mutations such as G497W have been linked to primary resistance, as this receptor variant undergoes a conformational change resulting in an obstruction of the antagonist entry site.

In an attempt to address resistance-related issues linked to available therapeutics, preclinical studies have delineated a number of additional SMO antagonists with promising pharmacological profiles, including the preclinical candidates PF-5274857, MRT-83, SANT-1 and MRT-92, as well as the clinically tested BMS-833923, CUR-61414, cyclopamine, saridegib/IPI-926, itraconazole, LEQ-506 and taladegib/LY-2940680. However, none of these molecules have as yet been clinically approved.

The extended repertoire of SMO mutations in BCCs found in the clinic in combination with the high prevalence of acquired resistance to existing inhibitors highlights the necessity of additional therapies effective against multiple SMO receptor variants. The compounds of the present invention have been identified as SMO inhibitors able to target a number of clinically relevant SMO mutations and could be a useful approach to treatment of BCC and/or other diseases/conditions associated with abnormal function of the hedgehog signalling pathway.

In addition to basal cell carcinoma and medulloblastic cancer, many other cancers are also associated with abnormal activation of the Hh signaling pathway, including esophageal cancer, gastric cancer, pancreatic cancer, and lung cancer. Moreover, studies have shown that the activity of the Hh signaling pathway is closely related to the problem of acquired resistance that can be problematic in various cancer treatments. For example, augmentation of the SMO gene and activation of the Hh pathway are considered to be one of the main reasons for the loss of therapeutic effect on the cell epidermal growth factor receptor (EGFR) inhibitor of non-small cell lung cancer. Therefore, the development of Hedgehog/SMO inhibitors as anticancer drugs to treat various cancers is very promising.

### THE INVENTION

The present invention provides compounds having activity as Smoothened (SMO) receptor antagonists. Provided is a compound of Formula (1a):

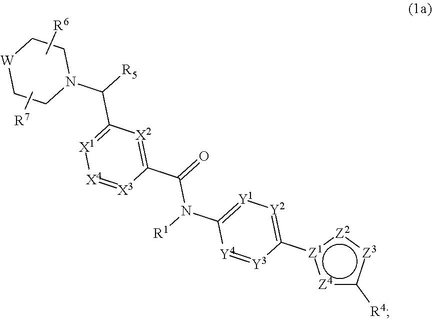

or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋₃ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted C₃₋₆ cycloalkyl, optionally substituted phenyl or an optionally substituted 5- or 6-membered heterocyclic ring;
wherein, when

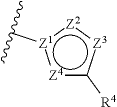

is:

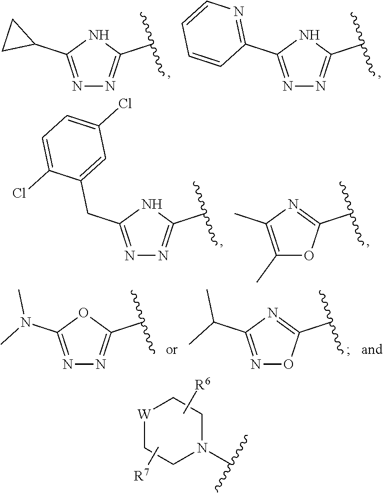

is

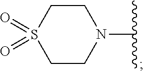 [SMILES: *N1CCS(=O)(=O)CC1]

X¹, X², X³, X⁴, Y¹, Y², Y³ and Y⁴ are not all CH, or R¹ and R⁵ are not both H.

Compounds of the present invention may be used as Smoothened (SMO) receptor modulators. Compounds of the present invention may be used as SMO inhibitors. Compounds of the present invention may be used as SMO antagonists. Compounds of the present invention may be used in the treatment of a disease or disorder characterised by activation of SMO receptors or by activation of the Hedgehog (Hh) signaling pathway. Compounds of the present invention may be used in the manufacture of medicaments. The compounds or medicaments may be for use in treating, preventing, ameliorating, controlling or reducing the risk of diseases or disorders in which SMO receptors are involved. Compounds of the present invention may be for use as a single agent or in combination with one or more additional pharmaceutical agents. Compounds of the present invention may be useful in the treatment of cancer.

Compounds of the present invention may be useful in the treatment of colorectal cancer, pancreatic cancer, breast cancer, prostate cancer, esophageal cancer, gastric cancer, blood cancer, lung cancer, brain cancer, medulloblastic cancer, skin cancer, basal cell carcinoma, head and neck cancer, ovarian cancer, bladder cancer, kidney cancer, lung cancer, breast cancer, pancreatic cancer and stomach cancer and symptoms related thereto.

### DETAILED DESCRIPTION OF THE INVENTION

The invention relates to novel compounds. The invention also relates to the use of novel compounds as modulators of SMO receptors, in particular as SMO receptor antagonists. The invention further relates to the use of novel compounds in the manufacture of medicaments for use as SMO antagonists. The invention further relates to compounds, compositions and medicaments that may be useful in the treatment of a disease or disorder characterised by activation of SMO receptors or by activation of the Hedgehog (Hh) signaling pathway, including cancer.

Provided is a compound of Formula (1a):

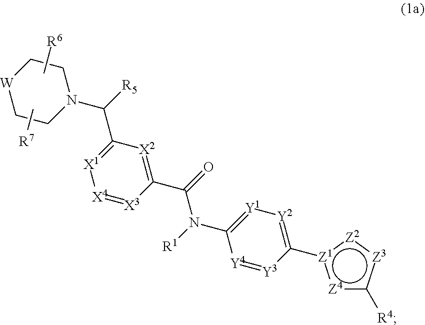

or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋₃ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted Ca.& cycloalkyl, optionally substituted phenyl or an optionally substituted 5- or 6-membered heterocyclic ring.

Provided is a compound of Formula (1a):

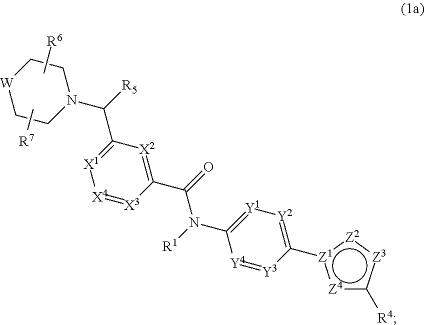

or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋ₛ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted C₃₋₆ cycloalkyl, optionally substituted phenyl or an optionally substituted 5- or 6-membered heterocyclic ring;
wherein, when

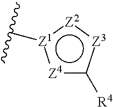

is:

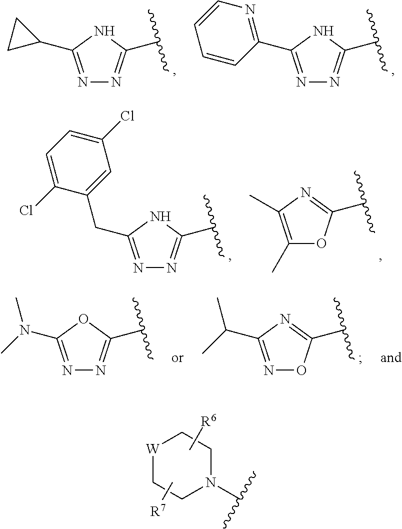

is

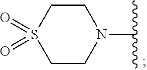 [SMILES: *N1CCS(=O)(=O)CC1]

X¹, X², X³, X⁴, Y¹, Y², Y³ and Y⁴ are not all CH, or R¹ and R⁵ are not both H.

Also provided is a compound of Formula (1a):

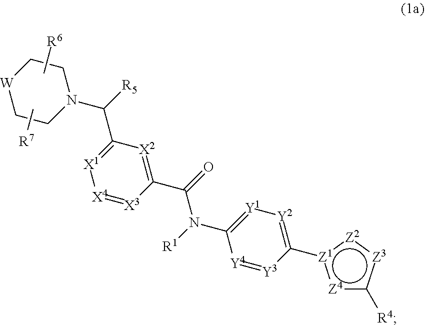

or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋₃ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted C₃a cycloalkyl, optionally substituted phenyl or an optionally substituted 5- or 6-membered heterocyclic ring;
wherein said compound is not:

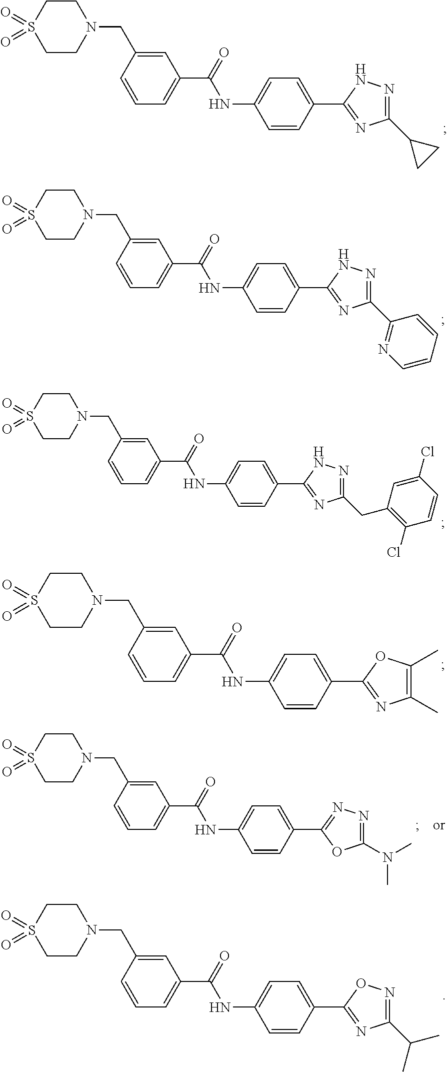 [SMILES: O=C(Nc1ccc(-c2nc(C3CC3)n[nH]2)cc1)c1cccc(CN2CCS(=O)(=O)CC2)c1 ; O=C(Nc1ccc(-c2nc(-c3ccccn3)n[nH]2)cc1)c1cccc(CN2CCS(=O)(=O)CC2)c1 ; O=C(Nc1ccc(-c2nc(Cc3cc(Cl)ccc3Cl)n[nH]2)cc1)c1cccc(CN2CCS(=O)(=O)CC2)c1 ; Cc1nc(-c2ccc(NC(=O)c3cccc(CN4CCS(=O)(=O)CC4)c3)cc2)oc1C ; CN(C)c1nnc(-c2ccc(NC(=O)c3cccc(CN4CCS(=O)(=O)CC4)c3)cc2)o1 ; CC(C)c1noc(-c2ccc(NC(=O)c3cccc(CN4CCS(=O)(=O)CC4)c3)cc2)n1]

Also provided is a compound of Formula (1a):

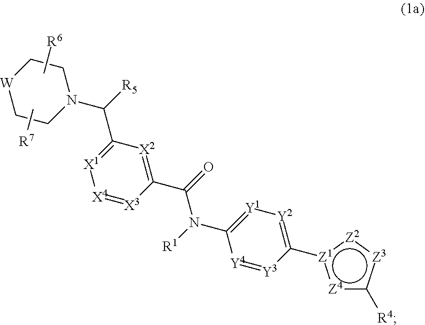

or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋₃ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted C₃₋₆ cycloalkyl, optionally substituted phenyl or an optionally substituted 5- or 6-membered heterocyclic ring;
wherein, when

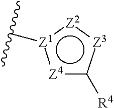

is:

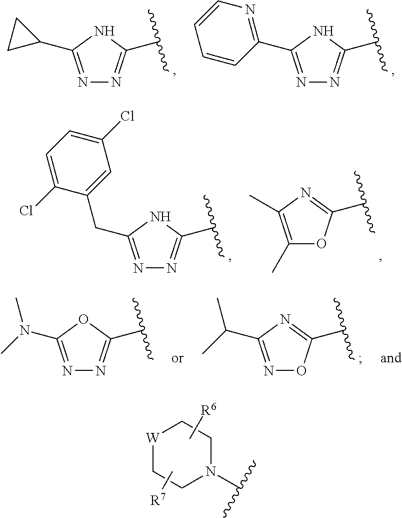

is

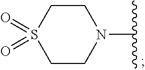 [SMILES: *N1CCS(=O)(=O)CC1]

one of X¹, X², X³ and X⁴ is N or CR², where R² is CN, halo, C₁₋₃ alkyl optionally substituted with 1-3 fluorine atoms or C₁₋₃ alkoxy optionally substituted with 1-3 fluorine atoms; and/or
one of Y¹, Y², Y³ and Y⁴ is N or CR³, where R³ is halo or methyl; and/or
R¹ is methyl; and/or
R⁵ is C₁₋₃ alkyl optionally substituted with 1-3 fluorine atoms.

In the compounds herein W can be S, SO or SO₂. W can be S. W can be SO. W can be SO₂.

In the compounds herein, the moiety:

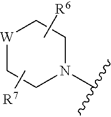

can be selected from:

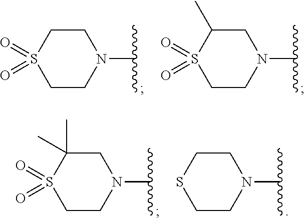

The moiety:

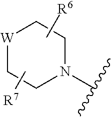

can be

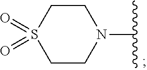 [SMILES: *N1CCS(=O)(=O)CC1]

In the compounds herein, X¹, X², X³ and X⁴ are N, CH or CR², where one or none of X¹, X², X³ and X⁴ are N and one or none of X¹, X², X³ and X⁴ are CR². X¹, X², X³ and X⁴ can all be CH. Three of X¹, X², X³ and X⁴ can be CH and one of X¹, X², X³ and X⁴ can be CR². Three of X¹, X², X³ and X⁴ can be CH and one of X¹, X², X³ and X⁴ can be CF or CCl. X¹ can be CH, CF, CCl or N. X² can be CH, CF, CCl or N. X³ can be CH, CF, CCl or N. X⁴ can be CH, CF, CCl or N.

The moiety:

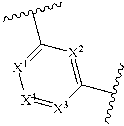

can be selected from:

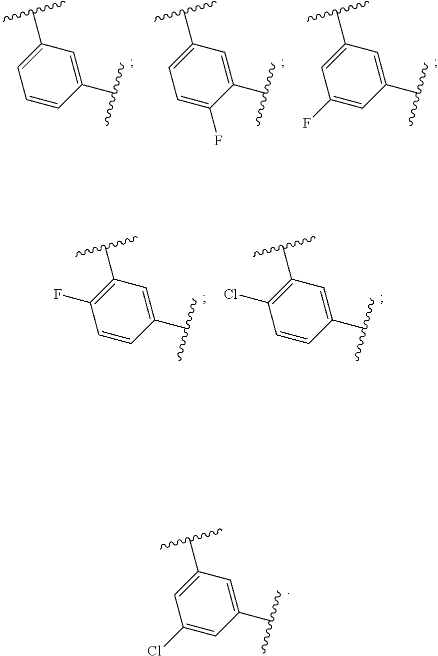 [SMILES: *c1cccc(*)c1 ; *c1ccc(F)c(*)c1 ; *c1cc(*)cc(F)c1 ; *c1ccc(F)c(*)c1 ; *c1ccc(Cl)c(*)c1 ; *c1cc(*)cc(Cl)c1]

In the compounds herein, Y¹, Y², Y³ and Y⁴ are N, CH or CR³, where one or none of Y¹, Y², Y³ and Y⁴ are N and one or none of Y, Y², Y³ and Y⁴ are CR³. Y¹, Y², Y³ and Y⁴ can all be CH. Three of Y, Y², Y³ and Y⁴ can be CH and one of Y¹, Y², Y³ and Y⁴ can be N. Y¹ can be CH or N. Y² can be CH or N. Y can be CH or N. Y⁴ can be CH or N.

The moiety:

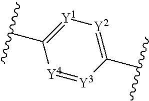

can be selected from:

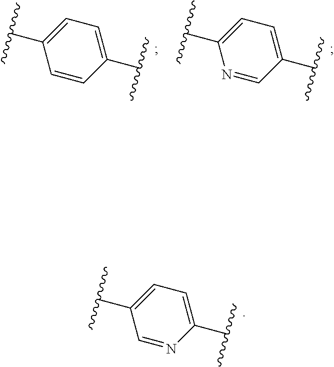 [SMILES: *c1ccc(*)cc1 ; ";" ; *c1ccc(*)nc1 ; ";" ; *c1ccc(*)nc1 ; "."]

In the compounds herein, Z¹ is C or N. Z¹ can be C. Z¹ can be N.

In the compounds herein, Z² is O, N, NR⁸, CR⁸ or S. Z² can be N, O, CH or NH. Z² can be N.

Z² can be O. Z² can be CH. Z² can be NH.

In the compounds herein, Z³ is O, N, NR⁹, CR⁹ or S. Z³ can be NH, N, CCH₃, O, NCH₃ or CH. Z³ can be NH. Z³ can be N. Z³ can be CCH₃. Z³ can be O. Z³ can be NCH₃. Z³ can be CH.

In the compounds herein, Z⁴ is O, N, NR¹⁰, CR¹⁰ or S. Z⁴ can be N, NH, CH, O or CCH₃. Z⁴ can be N. Z⁴ can be NH. Z⁴ can be CH. Z⁴ can be O. Z⁴ can be CCH₃.

In the compounds herein, the moiety:

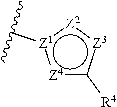

can be selected from:

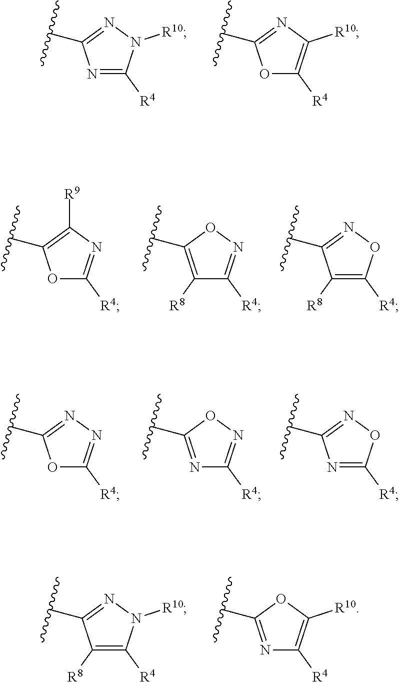

In the compounds herein, R¹ can be H or methyl. R¹ can be H. R¹ can be methyl.

In the compounds herein, R² can be H, CN, halo, C₁₋₃ alkyl optionally substituted with 1-3 fluorine atoms or C₁₋₃ alkoxy optionally substituted with 1-3 fluorine atoms. R² can be H, CN, halo, C₁₋₃ alkyl or C₁₋₃ alkoxy. R² can be H, methyl, Cl, F, methoxy, trifluoromethyl or CN. R² can be H, F or Cl. R² can be H. R² can be F. R² can be Cl.

In the compounds herein, R³ can be H, halo or methyl. R³ can be H, F, Cl or methyl. R³ can be H, methyl or F. R³ can be H. R³ can be F. R³ can be methyl.

In the compounds herein, R⁴ can be H, halo, NR¹¹R¹², a group —(CH₂)ₙR¹³, where n is 0-3, or linear or branched C₁₋₆ alkyl optionally substituted with 1-3 fluorine atoms.

R⁴ can be H, halo, NR¹¹R¹², a group —(CH₂)ₙR¹³, where n is 0-3, or linear or branched C₁₋₆ alkyl optionally substituted with 1-3 fluorine atoms;

R¹³ is C₃₋₆ cycloalkyl, phenyl optionally substituted with one or more R¹⁴ groups, a 5- or 6-membered heterocyclic group optionally substituted with one or more R¹⁴ groups, a 1,4-benzodioxane ring system optionally substituted with one or more R¹⁴ groups, or a 1,3 benzodioxole ring system optionally substituted with one or more R¹⁴ groups;
wherein R¹⁴ is halo, C₁₋₃ alkyl optionally substituted with 1-3 fluorine atoms, C₁₋₃ alkoxy optionally substituted with 1-3 fluorine atoms, or a group —(CH₂)ₘ(O)ₚR¹⁵;
where R¹⁵ is phenyl optionally substituted with 1-3 fluorine atoms or thiazole;
m is 0-3; and
p is 0 or 1.

In the compounds herein, R¹¹ and R¹² can independently be H or C₁₋₃ alkyl. R¹¹ and R¹² can independently be H or methyl. R¹¹ and R¹² can both be methyl. R¹¹ can be H or C₁₋₃ alkyl. R¹¹ can be H. R¹¹ can be methyl. R¹² can be H or C₁₋₃ alkyl. R¹² can be H. R¹² can be methyl.

In the compounds herein, R¹³ can be optionally substituted C₃₋₄ cycloalkyl, optionally substituted phenyl or an optionally substituted 5- or 6-membered heterocyclic ring.

R¹³ can be C₃₋₆ cycloalkyl, phenyl optionally substituted with one or more R¹⁴ groups, a 5- or 6-membered heterocyclic group optionally substituted with one or more R¹⁴ groups, a 1,4-benzodioxane ring system optionally substituted with one or more R¹⁴ groups, or a 1,3 benzodioxole ring system optionally substituted with one or more R¹⁴ groups.

R¹³ can be C₃₋₆ cycloalkyl, optionally substituted phenyl, an optionally substituted 5- or 6-membered heterocyclic group, an optionally substituted 1,4-benzodioxane ring system, or an optionally substituted 1,3 benzodioxole ring system;
wherein the optional substituents are selected from halo, C₁₋₃ alkyl optionally substituted with 1-3 fluorine atoms, C₁₋₃ alkoxy optionally substituted with 1-3 fluorine atoms and a group —(CH₂)ₘ(O)ₚR¹⁵.

R¹³ can be C₃₋₆ cycloalkyl, optionally substituted phenyl, an optionally substituted 5- or 6-membered heterocyclic group, an optionally substituted 1,4-benzodioxane ring system, or an optionally substituted 1,3 benzodioxole ring system;
wherein the optional substituents are selected from halo, C₁₋₃ alkyl optionally substituted with 1-3 fluorine atoms and C₁₋₃ alkoxy optionally substituted with 1-3 fluorine atoms.

R¹³ can be cyclopropyl, cyclobutyl, cyclopentyl or cyclohexyl.

In the compounds herein, R¹⁴ can be halo, C₁₋₃ alkyl optionally substituted with 1-3 fluorine atoms, C₁₋₃ alkoxy optionally substituted with 1-3 fluorine atoms, or a group —(CH₂)ₘ(O)ₚR¹⁵. R¹⁴ can be halo, C₁₋₃ alkyl optionally substituted with 1-3 fluorine atoms or C₁₋₃ alkoxy optionally substituted with 1-3 fluorine atoms.

R¹⁴ can be F, Cl, Br, methyl, CF₃, methoxy, phenyl, benzyl,

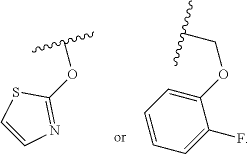 [SMILES: *Oc1nccs1 ; "or" ; *COc1ccccc1F]

In the compounds herein, R¹⁵ can be phenyl optionally substituted with 1-3 fluorine atoms or thiazole. R¹⁵ can be phenyl substituted with 1 fluorine atom or thiazole.

In the compounds herein, n can be 0-3. n can be 0. n can be 1. n can be 2. n can be 3.

In the compounds herein, m can be 0-3. m can be 0. m can be 1. m can be 2. m can be 3.

In the compounds herein, p can be 0 or 1. p can be 0. p can be 1.

R⁴ can be selected from:

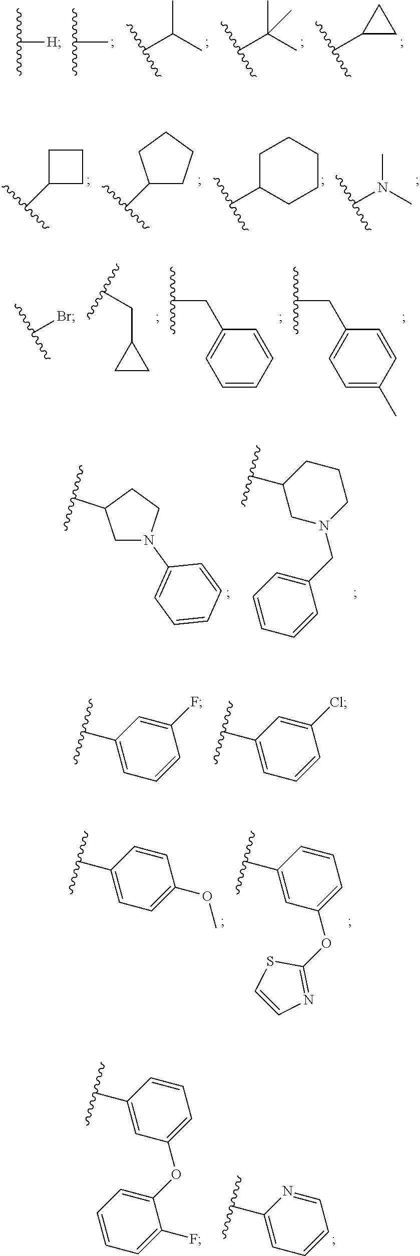

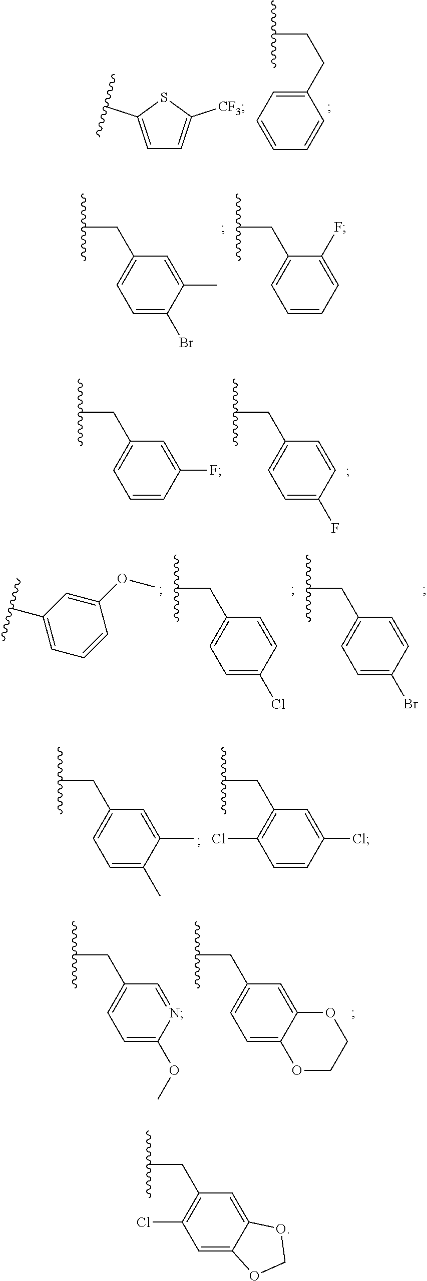

In the compounds herein, the moiety:

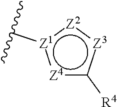

can be selected from:

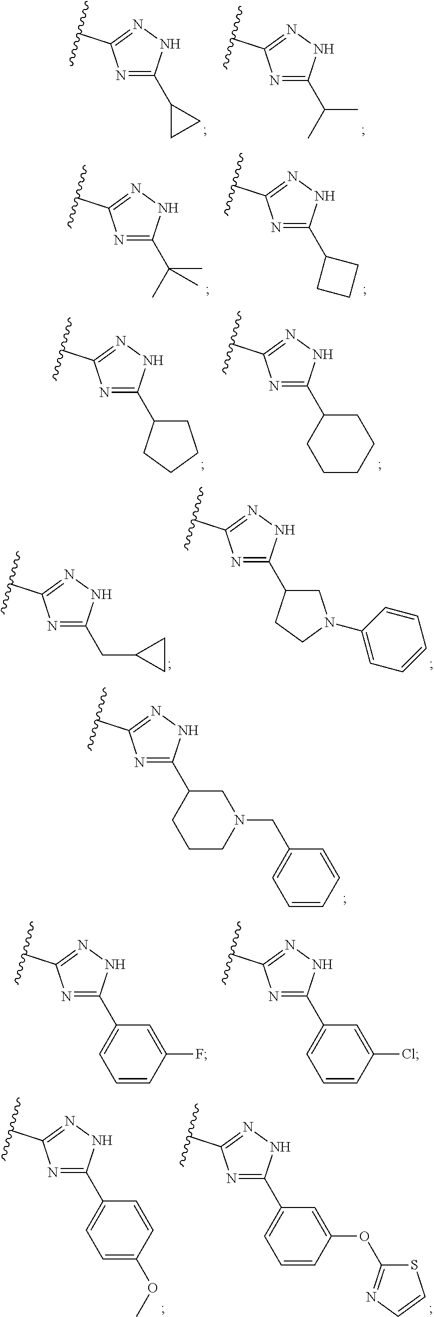

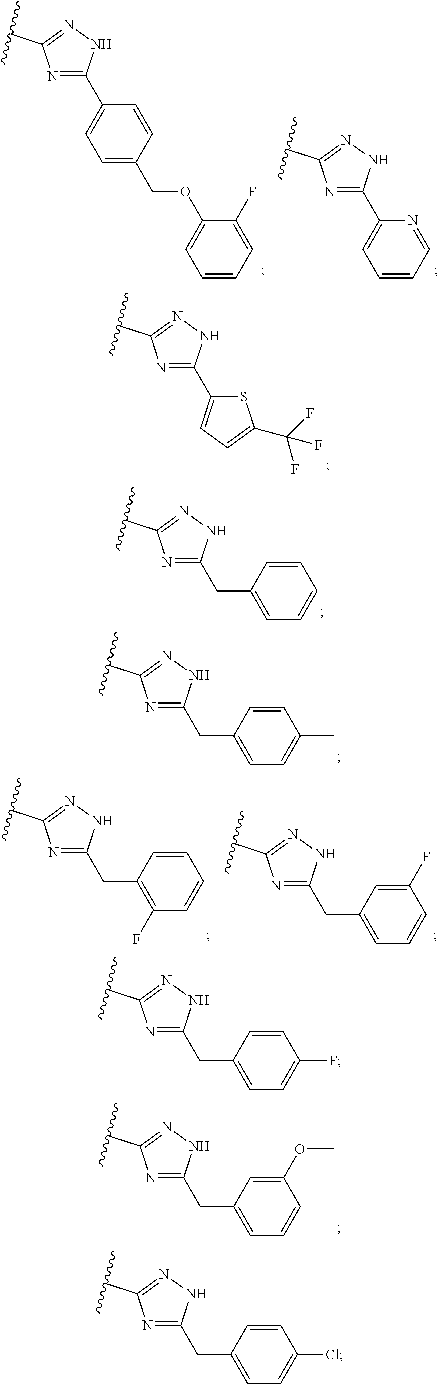

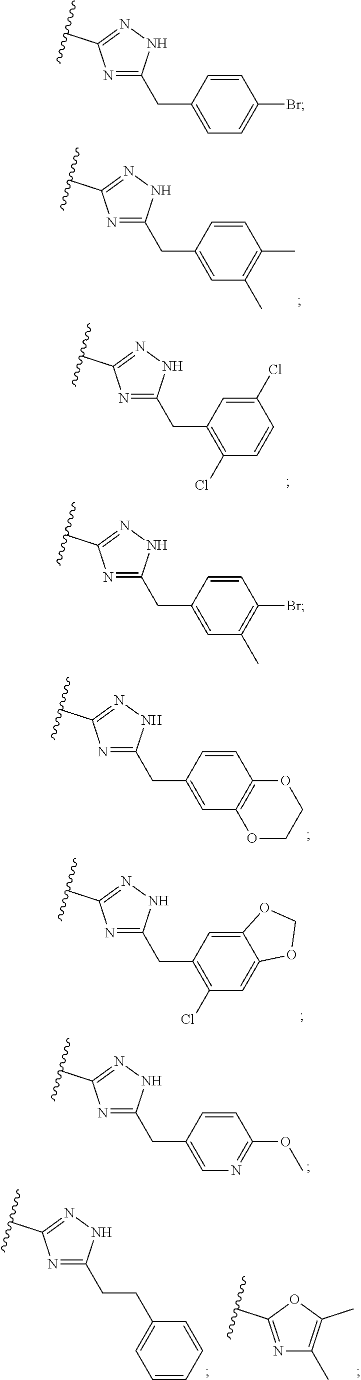

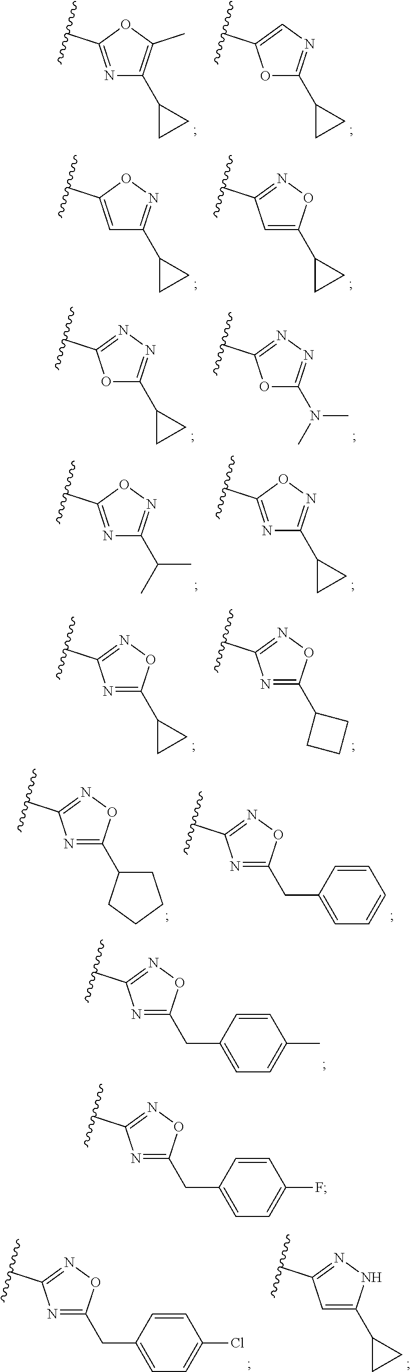

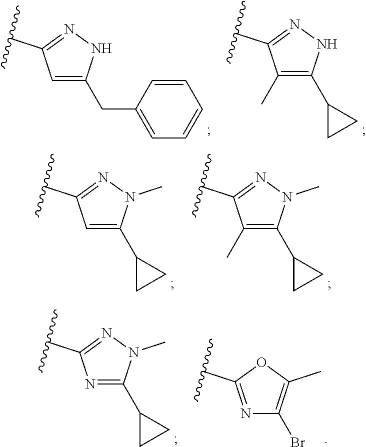

The moiety:

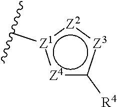

can be selected from:

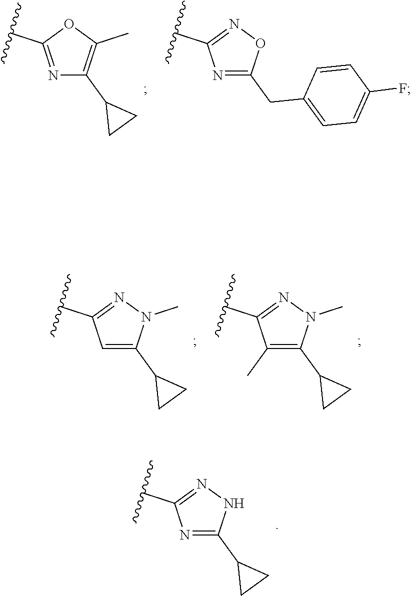

In the compounds herein, R⁵, R⁶ and R⁷ can independently be H or C₁₋₃ alkyl optionally substituted with 1-3 fluorine atoms. R⁵, R⁶ and R⁷ can independently be H or C₁₋₃ alkyl. R⁵, R⁶ and R⁷ can independently be H or methyl. R⁵, R⁶ and R⁷ can all be H.

R⁵ can be H or methyl. R⁵ can be H. R⁵ can be methyl.

R⁶ can be H or methyl. R⁶ can be H. R⁶ can be methyl.

R⁷ can be H or methyl. R⁷ can be H. R⁷ can be methyl.

In the compounds herein, R⁶, R⁹ and R¹⁰ can independently be H or C₁₋₃ alkyl optionally substituted with 1-3 fluorine atoms. R⁶, R⁹ and R¹⁰ can independently be H or C₁₋₃ alkyl. R⁶, R⁹ and R¹⁰ can independently be H or methyl.

R⁶ can be H or methyl. R⁶ can be H. R⁶ can be methyl.

R⁹ can be H or methyl. R⁹ can be H. R⁹ can be methyl.

R¹⁰ can be H or methyl. R¹⁰ can be H. R¹⁰ can be methyl.

In the compounds herein, when

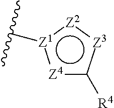

is:

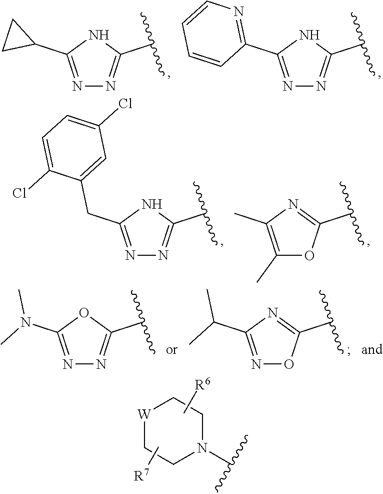

is

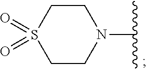 [SMILES: *N1CCS(=O)(=O)CC1]

X¹, X², X³, X⁴, Y¹, Y², Y³ and Y⁴ are not all CH, or R¹ and R⁵ are not both H.

For example, when

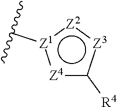

is:

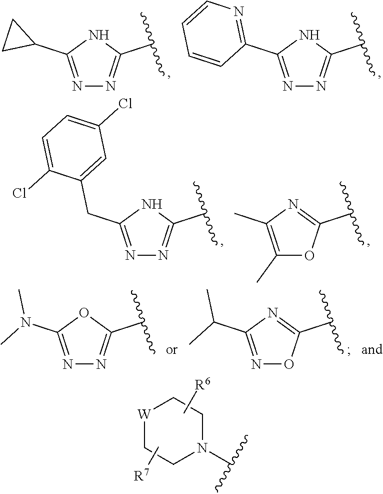

is

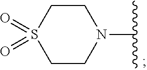 [SMILES: *N1CCS(=O)(=O)CC1]

one of X¹, X², X³ and X⁴ is N or CR², where R² is not H; and/or
one of Y¹, Y², Y³ and Y⁴ is N or CR³, where R³ is not H; and/or
one of R¹ and R⁵ is not H.

For example, when

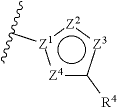

is:

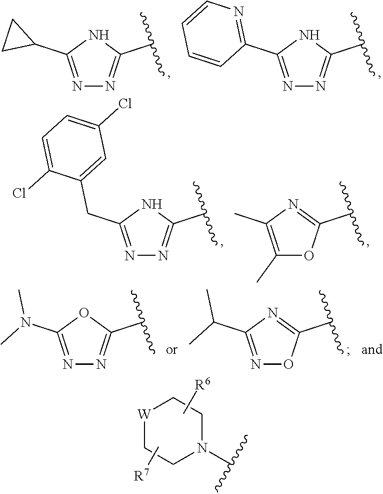

is

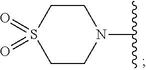 [SMILES: *N1CCS(=O)(=O)CC1]

one of X¹, X², X³ and X⁴ is N or CR², where R² is CN, halo, C₁₋₃ alkyl optionally substituted with 1-3 fluorine atoms or C₁₋₃ alkoxy optionally substituted with 1-3 fluorine atoms; and/or
one of Y¹, Y², Y³ and Y⁴ is N or CR³, where R³ is halo or methyl; and/or
R¹ is methyl; and/or
R⁵ is C₁₋₃ alkyl optionally substituted with 1-3 fluorine atoms.

For example, when

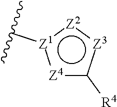

is:

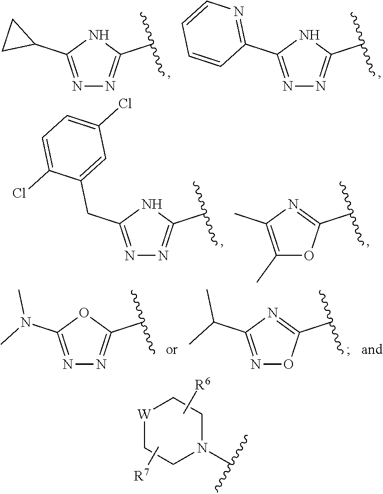

is:

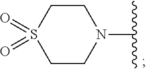 [SMILES: *N1CCS(=O)(=O)CC1]

one of X¹, X², X³ and X⁴ is N, CF or CCl; and/or
one of Y¹, Y², Y³ and Y⁴ is N; and/or
one of R¹ and R⁵ is methyl.

The compound can be a compound of formula (2a):

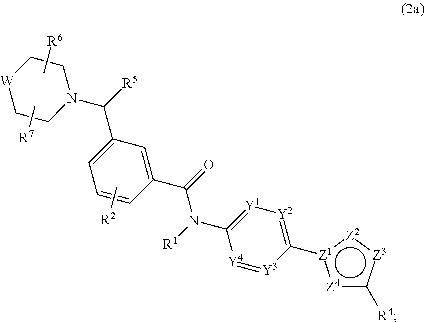

or a salt thereof.

The compound can be a compound of formula (2b), (2c) or (2d):

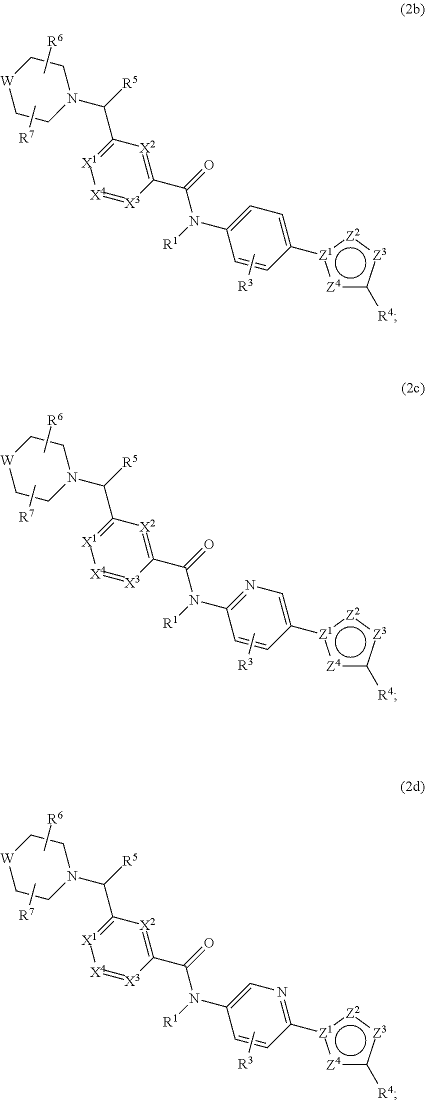

or a salt thereof.

The compound can be a compound of formula (3a), (3b) or (3c):

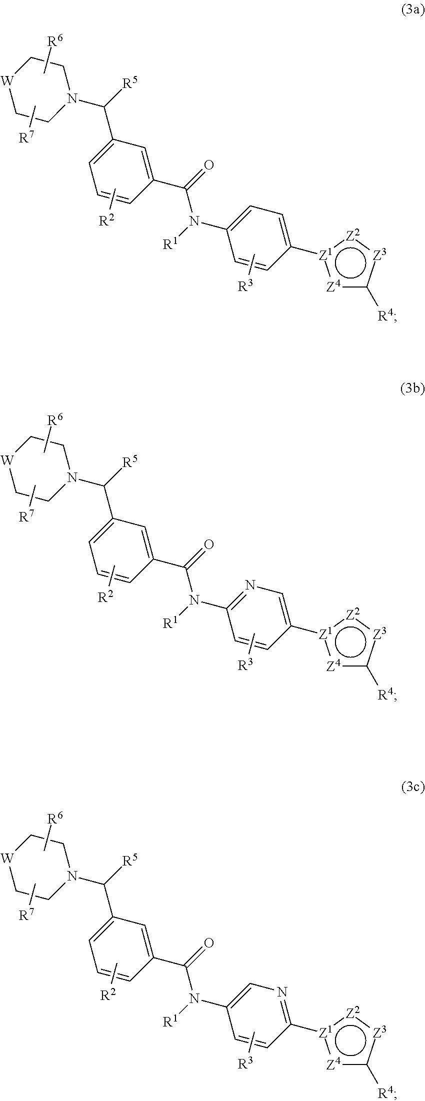

or a salt thereof.

The compound can be a compound of Formula (4a), (4b) or (4c):

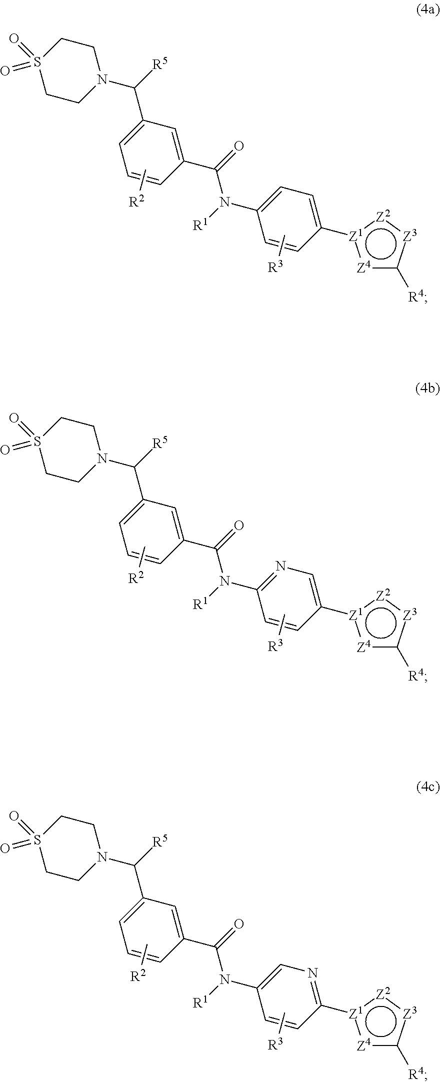

a salt thereof.

The compound can be a compound of Formula (4ai), (4bi) or (4ci):

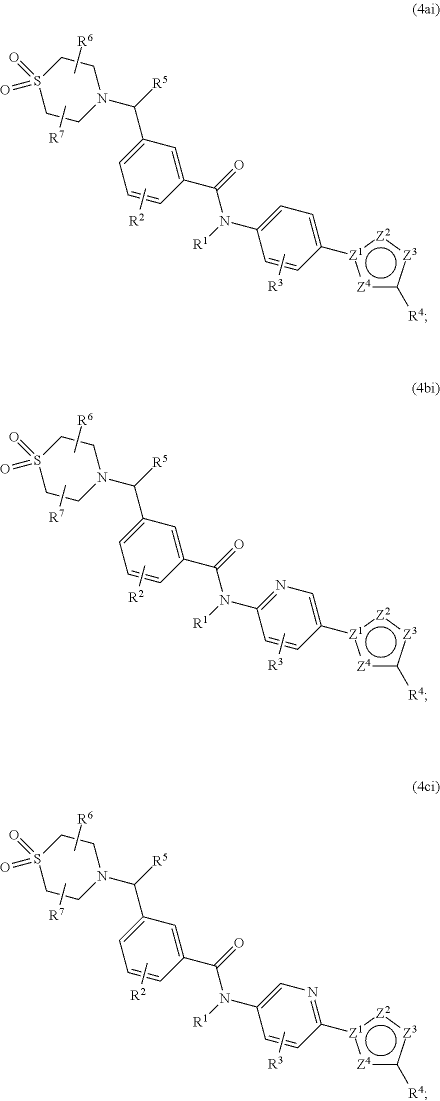

or a salt thereof.

The compound can be a compound of Formula (4d), (4e) or (4f):

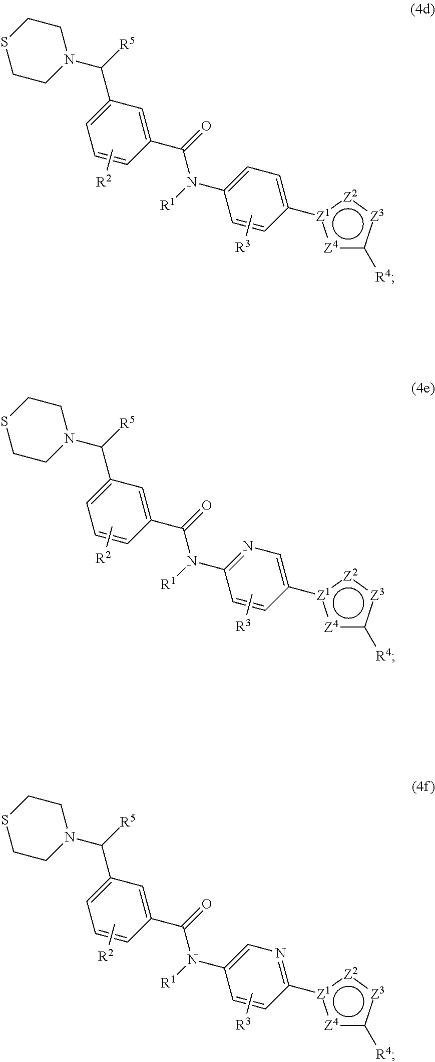

or a salt thereof.

The compound can be a compound of Formula (4di), (4ei) or (4fi):

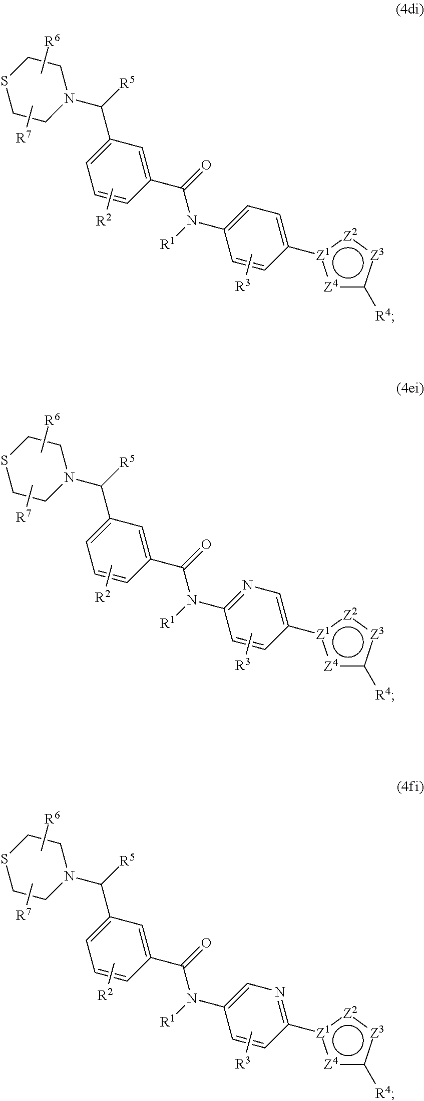

or a salt thereof.

The compound can be a compound of Formula (5a), (5b), (5c), (5d) or (5e):

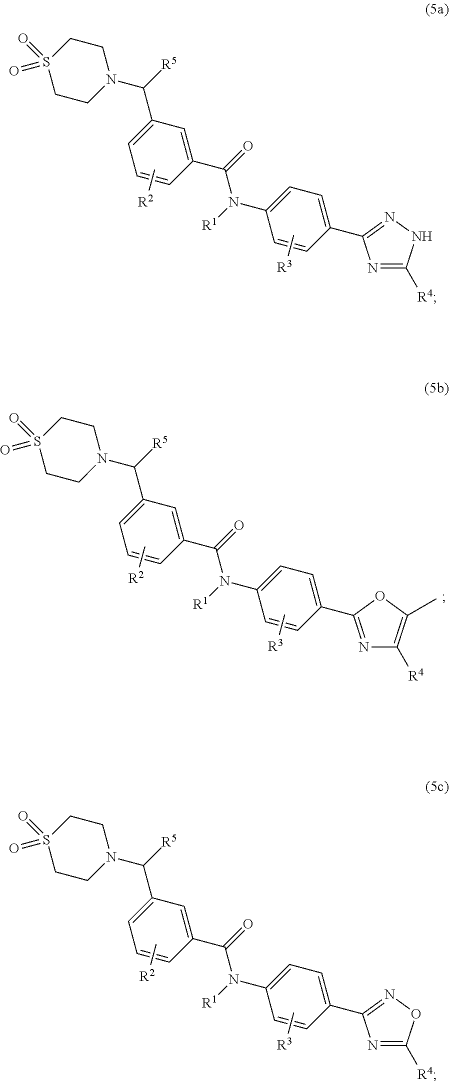

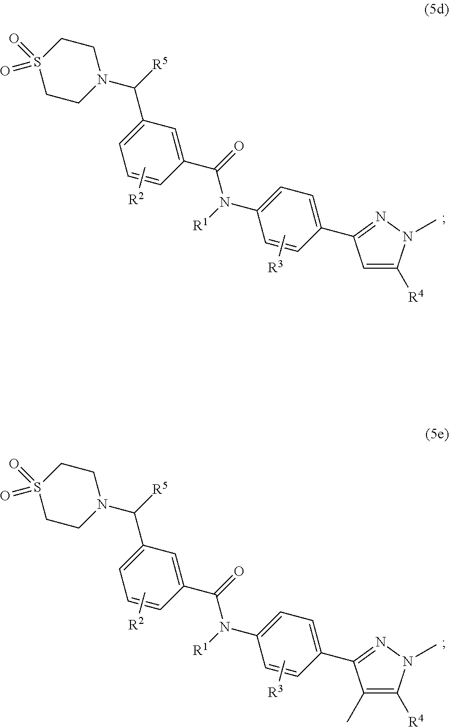

or a salt thereof.

The compound can be a compound of Formula (5ai), (5bi), (5ci), (5di) or (5ei):

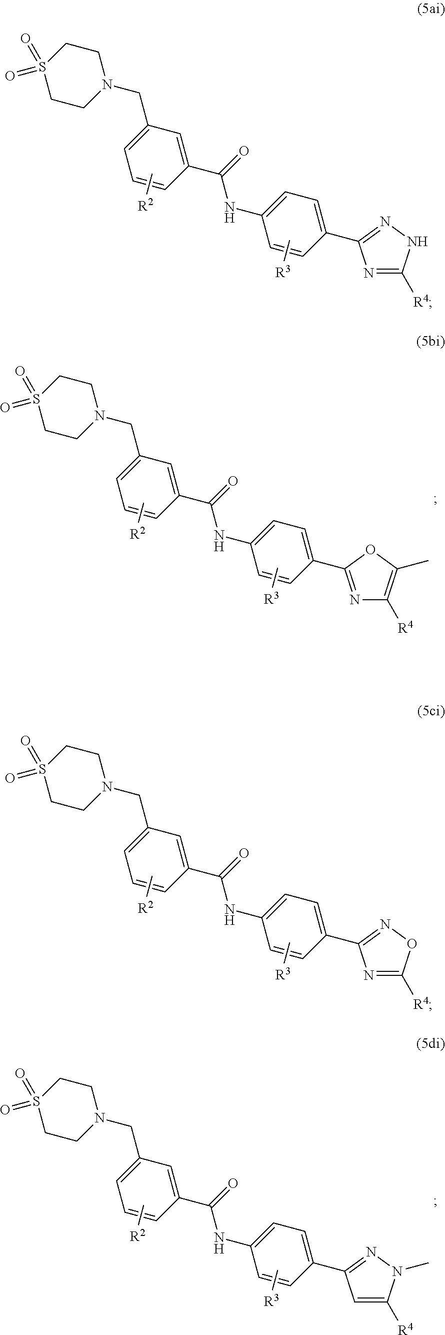

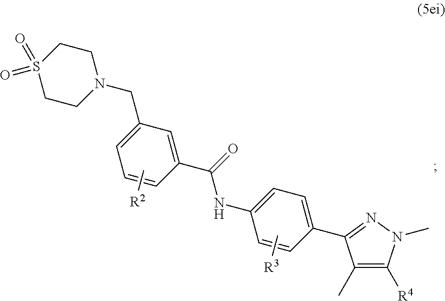

or a salt thereof.

The compound can be a compound of Formula (6a), (6b) or (6c):

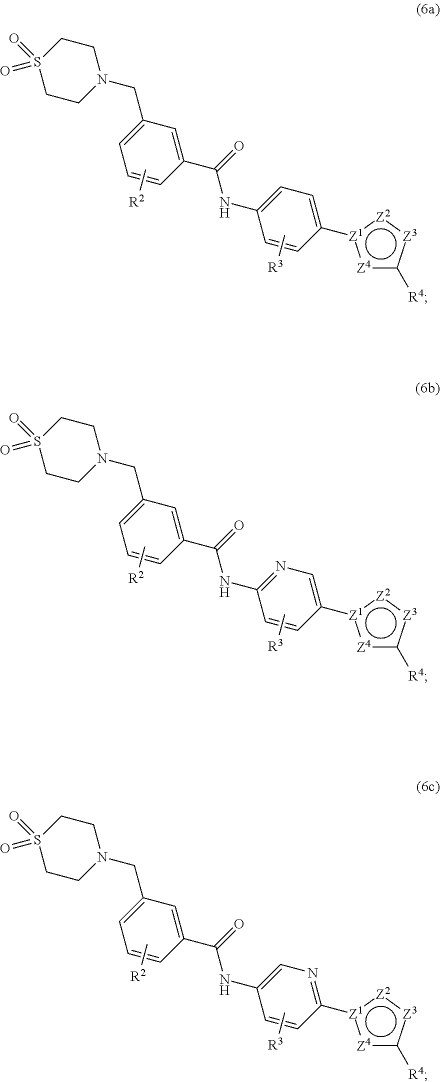

or a salt thereof.

The compound can be selected from any one of Examples 1-1 to 5-5 as shown in Table 1 or a salt thereof.

The compound can be selected from the group consisting of:
N-[4-(3-Cyclopropyl-1H-1,2,4-triazol-5-yl)phenyl]-3-(thiomorpholin-4-ylmethyl)benzamide;
N-[4-(3-Cyclopropyl-1H-1,2,4-triazol-5-yl)phenyl]-3-[(2-methyl-1,1-dioxo-1,4-thiazinan-4-yl)methyl]benzamide;
N-[4-(3-Cyclopropyl-1H-1,2,4-triazol-5-yl)phenyl]-3-[(2,2-dimethyl-1,1-dioxo-1,4-thiazinan-4-yl)methyl]benzamide;
N-[4-(5-Cyclopropyl-1H-1,2,4-triazol-3-yl)phenyl]-5-[(1,1-dioxo-1,4-thiazinan-4-yl)methyl]-2-fluorobenzamide;
N-[4-(5-Cyclopropyl-1H-1,2,4-triazol-3-yl)phenyl]-3-[(1,1-dioxo-1,4-thiazinan-4-yl)methyl]-5-fluorobenzamide;
N-[4-(5-Cyclopropyl-1H-1,2,4-triazol-3-yl)phenyl]-3-[(1,1-dioxo-1,4-thiazinan-4-yl)methyl]-4-fluorobenzamide;
3-Chloro-N-[4-(5-cyclopropyl-1H-1,2,4-triazol-3-yl)phenyl]-5-[(1,1-dioxo-1,4-thiazinan-4-yl)methyl]benzamide;
4-Chloro-N-[4-(5-cyclopropyl-1H-1,2,4-triazol-3-yl)phenyl]-3-[(1,1-dioxo-1,4-thiazinan-4-yl)methyl]benzamide;
3-[(1,1-Dioxo-1,4-thiazinan-4-yl)methyl]-N-[4-(3-propan-2-yl-1H-1,2,4-triazol-5-yl)phenyl]benzamide;
N-[4-(5-tert-Butyl-1H-1,2,4-triazol-3-yl)phenyl]-3-[(1,1-dioxo-1,4-thiazinan-4-yl)methyl]benzamide;
N-[4-(3-Cyclopropyl-1H-1,2,4-triazol-5-yl)phenyl]-3-[(1,1-dioxo-1,4-thiazinan-4-yl)methyl]benzamide;
N-[4-(3-Cyclobutyl-1H-1,2,4-triazol-5-yl)phenyl]-3-[(1,1-dioxo-1,4-thiazinan-4-yl)methyl]benzamide;
N-[4-(3-Cyclopentyl-1H-1,2,4-triazol-5-yl)phenyl]-3-[(1,1-dioxo-1,4-thiazinan-4-yl)methyl]benzamide;
N-[4-(3-Cyclohexyl-1H-1,2,4-triazol-5-yl)phenyl]-3-[(1,1-dioxo-1,4-thiazinan-4-yl)methyl]benzamide;
N-[4-[3-(Cyclopropylmethyl)-1H-1,2,4-triazol-5-yl]phenyl]-3-[(1,1-dioxo-1,4-thiazinan-4-yl)methyl]benzamide;
3-[(1,1-Dioxo-1,4-thiazinan-4-yl)methyl]-N-[4-[3-(1-phenylpyrrolidin-3-yl)-1H-1,2,4-triazol-5-yl]phenyl]benzamide;
N-[4-[3-(1-Benzylpiperidin-3-yl)-1H-1,2,4-triazol-5-yl]phenyl]-3-[(1,1-dioxo-1,4-thiazinan-4-yl)methyl]benzamide;
3-[(1,1-Dioxo-1,4-thiazinan-4-yl)methyl]-N-[4-[3-(3-fluorophenyl)-1H-1,2,4-triazol-5-yl]phenyl]benzamide;
N-[4-[3-(3-Chlorophenyl)-1H-1,2,4-triazol-5-yl]phenyl]-3-[(1,1-dioxo-1,4-thiazinan-4-yl)methyl]benzamide;
3-[(1,1-Dioxo-1,4-thiazinan-4-yl)methyl]-N-[4-[5-(4-methoxyphenyl)-1H-1,2,4-triazol-3-yl]phenyl]benzamide;
3-[(1,1-Dioxo-1,4-thiazinan-4-yl)methyl]-N-[4-[3-[3-(1,3-thiazol-2-yloxy)phenyl]-1H-1,2,4-triazol-5-yl]phenyl]benzamide;
3-[(1,1-Dioxo-1,4-thiazinan-4-yl)methy]-N-[4-[3-[4-[(2-fluorophenoxy)methyl]phenyl]-1H-1,2,4-triazol-5-yl]phenyl]benzamide;
3-[(1,1-Dioxo-1,4-thiazinan-4-yl)methyl]-N-[4-(3-pyridin-2-yl-1H-1,2,4-triazol-5-yl)phenyl]benzamide;
3-[(1,1-Dioxo-1,4-thiazinan-4-yl)methyl]-N-[4-[3-[5-(trifluoromethyl)thiophen-2-yl]-1H-1,2,4-triazol-5-yl]phenyl]benzamide;
N-[4-(3-Benzyl-1H-1,2,4-triazol-5-yl)phenyl]-3-[(1,1-dioxo-1,4-thiazinan-4-yl)methyl]benzamide;
3-[(1,1-Dioxo-1,4-thiazinan-4-yl)methyl]-N-[4-[5-[(4-methylphenyl)methyl]-1H-1,2,4-triazol-3-yl]phenyl]benzamide;
3-[(1,1-Dioxo-1,4-thiazinan-4-yl)methy]-N-[4-[3-[(2-fluorophenyl)methyl]-1H-1,2,4-triazol-5-yl]phenyl]benzamide;
3-[(1,1-Dioxo-1,4-thiazinan-4-yl)methy]-N-[4-[3-[(3-fluorophenyl)methyl]-1H-1,2,4-triazol-5-yl]phenyl]benzamide;
3-[(1,1-Dioxo-1,4-thiazinan-4-yl)methy]-N-[4-[3-[(4-fluorophenyl)methyl]-1H-1,2,4-triazol-5-yl]phenyl]benzamide;
3-[(1,1-Dioxo-1,4-thiazinan-4-yl)methy]-N-[4-[5-[(3-methoxyphenyl)methyl]-1H-1,2,4-triazol-3-yl]phenyl]benzamide;
N-[4-[3-[(4-Chlorophenyl)methyl]-1H-1,2,4-triazol-5-yl]phenyl]-3-[(1,1-dioxo-1,4-thiazinan-4-yl)methyl]benzamide;
N-[4-[3-[(4-bromophenyl)methy]-1H-1,2,4-triazol-5-yl]phenyl]-3-[(1,1-dioxo-1,4-thiazinan-4-yl)methyl]benzamide;
N-[4-[5-[(3,4-Dimethylphenyl)methyl]-1H-1,2,4-triazol-3-yl]phenyl]-3-[(1,1-dioxo-1,4-thiazinan-4-yl)methyl]benzamide;
N-[4-[3-[(2,5-Dichlorophenyl)methyl]-1H-1,2,4-triazol-5-yl]phenyl]-3-[(1,1-dioxo-1,4-thiazinan-4-yl)methyl]benzamide;
N-[4-[5-[(4-Bromo-3-methylphenyl)methyl]-1H-1,2,4-triazol-3-yl]phenyl]-3-[(1,1-dioxo-1,4-thiazinan-4-yl)methyl]benzamide;
N-[4-[3-(2,3-Dihydro-1,4-benzodioxin-6-ylmethyl)-1H-1,2,4-triazol-5-yl]phenyl]-3-[(1,1-dioxo-1,4-thiazinan-4-yl)methyl]benzamide;
N-[4-[3-[(6-Chloro-1,3-benzodioxol-5-yl)methyl]-1H-1,2,4-triazol-5-yl]phenyl]-3-[(1,1-dioxo-1,4-thiazinan-4-yl)methyl]benzamide;
3-[(1,1-Dioxo-1,4-thiazinan-4-yl)methyl]-N-[4-[5-[(6-methoxypyridin-3-yl)methyl]-1H-1,2,4-triazol-3-yl]phenyl]benzamide;
3-[(1,1-Dioxo-1,4-thiazinan-4-yl)methyl]-N-[4-[3-(2-phenylethyl)-1H-1,2,4-triazol-5-yl]phenyl]benzamide;
N-[4-(4,5-Dimethyl-1,3-oxazol-2-yl)phenyl]-3-[(1,1-dioxo-1,4-thiazinan-4-yl)methyl]benzamide;
N-[4-(4-Cyclopropyl-5-methyl-1,3-oxazol-2-yl)phenyl]-3-[(1,1-dioxo-1,4-thiazinan-4-yl)methyl]benzamide;
N-[4-(4-Cyclopropyl-5-methyl-1,3-oxazol-2-yl)phenyl]-3-[(1,1-dioxo-1,4-thiazinan-4-yl)methyl]-4-fluorobenzamide;
4-Chloro-N-[4-(4-cyclopropyl-5-methyl-1,3-oxazol-2-yl)phenyl]-3-[(1,1-dioxo-1,4-thiazinan-4-yl)methyl]benzamide;
N-[4-(2-Cyclopropyl-1,3-oxazol-5-yl)phenyl]-3-[(1,1-dioxo-1,4-thiazinan-4-yl)methyl]benzamide;
N-[4-(5-Cyclopropyl-1,2-oxazol-3-yl)phenyl]-3-[(1,1-dioxo-1,4-thiazinan-4-yl)methyl]benzamide;
N-[4-(5-Cyclopropyl-1,2-oxazol-3-yl)phenyl]-3-[(1,1-dioxo-1,4-thiazinan-4-yl)methyl]benzamide;
N-[4-(5-Cyclopropyl-1,3,4-oxadiazol-2-yl)phenyl]-3-[(1,1-dioxo-1,4-thiazinan-4-yl)methyl]benzamide;
N-[4-[5-(Dimethylamino)-1,3,4-oxadiazol-2-yl]phenyl]-3-[(1,1-dioxo-1,4-thiazinan-4-yl)methyl]benzamide;
3-[(1,1-Dioxo-1,4-thiazinan-4-yl)methyl]-N-[4-(3-propan-2-yl-1,2,4-oxadiazol-5-yl)phenyl]benzamide;
N-[4-(3-Cyclopropyl-1,2,4-oxadiazol-5-yl)phenyl]-3-[(1,1-dioxo-1,4-thiazinan-4-yl)methyl]benzamide;
N-[4-(5-Cyclopropyl-1,2,4-oxadiazol-3-yl)phenyl]-3-[(1,1-dioxo-1,4-thiazinan-4-yl)methyl]benzamide;
N-[4-(5-Cyclobutyl-1,2,4-oxadiazol-3-yl)phenyl]-3-[(1,1-dioxo-1,4-thiazinan-4-yl)methyl]benzamide;
N-[4-(5-Cyclopentyl-1,2,4-oxadiazol-3-yl)phenyl]-3-[(1,1-dioxo-1,4-thiazinan-4-yl)methyl]benzamide;
N-[4-(5-Benzyl-1,2,4-oxadiazol-3-yl)phenyl]-3-[(1,1-dioxo-1,4-thiazinan-4-yl)methyl]benzamide;
3-[(1,1-Dioxo-1,4-thiazinan-4-yl)methyl]-N-[4-[5-[(4-methylphenyl)methyl]-1,2,4-oxadiazol-3-yl]phenyl]benzamide;
3-[(1,1-Dioxo-1,4-thiazinan-4-yl)methyl]-N-[4-[5-[(4-fluorophenyl)methyl]-1,2,4-oxadiazol-3-yl]phenyl]benzamide;
N-[4-[5-[(4-Chlorophenyl)methyl]-1,2,4-oxadiazol-3-yl]phenyl]-3-[(1,1-dioxo-1,4-thiazinan-4-yl)methyl]benzamide;
N-[4-(5-Cyclopropyl-1H-pyrazol-3-yl)phenyl]-3-[(1,1-dioxo-1,4-thiazinan-4-yl)methyl]benzamide;
4-Chloro-N-[4-(5-cyclopropyl-1H-pyrazol-3-yl)phenyl]-3-[(1,1-dioxo-1,4-thiazinan-4-yl)methyl]benzamide;
N-[4-(3-Benzyl-1H-pyrazol-5-yl)phenyl]-3-[(1,1-dioxo-1,4-thiazinan-4-yl)methyl]benzamide;
4-Chloro-N-[4-(5-cyclopropyl-4-methyl-1H-pyrazol-3-yl)phenyl]-3-[(1,1-dioxo-1,4-thiazinan-4-yl)methyl]benzamide;
N-[4-(5-Cyclopropyl-2-methylpyrazol-3-yl)phenyl]-3-[(1,1-dioxo-1,4-thiazinan-4-yl)methyl]benzamide;
4-Chloro-N-[4-(5-cyclopropyl-1-methylpyrazol-3-yl)phenyl]-3-[(1,1-dioxo-1,4-thiazinan-4-yl)methyl]benzamide;
N-[4-(5-Cyclopropyl-1,4-dimethylpyrazol-3-yl)phenyl]-3-[(1,1-dioxo-1,4-thiazinan-4-yl)methyl]benzamide;
4-Chloro-N-[4-(5-cyclopropyl-1,4-dimethylpyrazol-3-yl)phenyl]-3-[(1,1-dioxo-1,4-thiazinan-4-yl)methyl]benzamide;
N-[4-(5-Cyclopropyl-1-methyl-1,2,4-triazol-3-yl)phenyl]-3-[(1,1-dioxo-1,4-thiazinan-4-yl)methyl]benzamide;
N-[6-(4-Bromo-5-methyl-1,3-oxazol-2-yl)pyridin-3-yl]-4-chloro-3-[(1,1-dioxo-1,4-thiazinan-4-yl)methyl]benzamide;
4-Chloro-N-[6-(4-cyclopropyl-5-methyl-1,3-oxazol-2-yl)pyridin-3-yl]-3-[(1,1-dioxo-1,4-thiazinan-4-yl)methyl]benzamide;
4-Chloro-N-[6-(5-cyclopropyl-1H-pyrazol-3-yl)pyridin-3-yl]-3-[(1,1-dioxo-1,4-thiazinan-4-yl)methyl]benzamide;
N-[6-(5-Cyclopropyl-1-methylpyrazol-3-yl)pyridin-3-yl]-3-[(1,1-dioxo-1,4-thiazinan-4-yl)methyl]benzamide;
N-[5-(5-Cyclopropyl-1-methylpyrazol-3-yl)pyridin-2-yl]-3-[(1,1-dioxo-1,4-thiazinan-4-yl)methyl]benzamide;
   or a salt thereof.

The salt thereof can be a pharmaceutically acceptable salt. The salt thereof can be an HCl salt. The compound can be selected from:
3-[(1,1-Dioxo-1,4-thiazinan-4-yl)methyl]-N-[4-(3-propan-2-yl-1H-1,2,4-triazol-5-yl)phenyl]benzamide HCl salt;
N-[4-(3-Cyclobutyl-1H-1,2,4-triazol-5-yl)phenyl]-3-[(1,1-dioxo-1,4-thiazinan-4-yl)methyl]benzamide HCl salt;
N-[4-(3-Benzyl-1H-1,2,4-triazol-5-yl)phenyl]-3-[(1,1-dioxo-1,4-thiazinan-4-yl)methyl]benzamide HCl salt;
N-[4-(5-Cyclopropyl-1,3,4-oxadiazol-2-yl)phenyl]-3-[(1,1-dioxo-1,4-thiazinan-4-yl)methyl]benzamide HCl salt;
N-[4-(3-Cyclopropyl-1,2,4-oxadiazol-5-yl)phenyl]-3-[(1,1-dioxo-1,4-thiazinan-4-yl)methyl]benzamide HCl salt;
N-[4-(5-Cyclopropyl-1,2,4-oxadiazol-3-yl)phenyl]-3-[(1,1-dioxo-1,4-thiazinan-4-yl)methyl]benzamide HCl salt;
N-[4-(5-Cyclopropyl-1-methyl-1,2,4-triazol-3-yl)phenyl]-3-[(1,1-dioxo-1,4-thiazinan-4-yl)methyl]benzamide HCl salt.

Further embodiments of the invention include the use of a compound of Formula (1a) or a salt thereof or a pharmaceutical composition comprising a compound of Formula (1a) as a SMO receptor modulator. Compounds of the present invention may be used as SMO receptor modulators. Compounds of the present invention may be used as SMO receptor inhibitors or antagonists. Compounds of the present invention may be used in the treatment of a disease or disorder characterised by activation of SMO receptors or by activation of the Hedgehog (Hh) signaling pathway.

Compounds of the present invention may be used in the treatment of cancer or symptoms related thereto. Compounds of the present invention may be used in the treatment of colorectal cancer, pancreatic cancer, breast cancer, prostate cancer, esophageal cancer, gastric cancer, blood cancer, lung cancer, brain cancer, medulloblastic cancer, skin cancer, basal cell carcinoma, head and neck cancer, ovarian cancer, bladder cancer, kidney cancer, lung cancer, breast cancer, pancreatic cancer and stomach cancer and symptoms related thereto.

In some embodiments, compounds and compositions detailed herein are used as modulators of SMO receptors. Provided herein is a method of treating a disease in an individual comprising administering an effective amount of a compound of Formula (1a) or any embodiment, variation or aspect thereof.

Compounds as SMO receptor modulators as disclosed herein can be useful as a prophylactic or therapeutic agent for SMO associated diseases such as cancer:

In some embodiments, a compound or salt thereof described herein or a composition described herein may be used in a method of treating cancer in an individual, wherein the individual has had a prior treatment. In some embodiments, the cancer is resistant or refractory to the prior treatment. In some embodiments, the cancer has progressed on the prior treatment. The cancer may be recurrent cancer.

In some embodiments, the administration of the compound, salt, or composition reduces tumor growth, tumor proliferation, or tumorigenicity in the individual. In some embodiments, the compound, salt, or composition may be used in a method of reducing tumor growth, tumor proliferation, or tumorigenicity in an individual in need thereof. In some embodiments, tumor growth is slowed or arrested. In some embodiments, tumor growth is reduced at least about 10%, 20%, 30%, 40%, 50%, 60%, 70%, 80% k, or 90%. In some embodiments, the tumor is reduced in size. In some embodiments, tumor size is reduced at least about 10%, 20%, 30%, 40%, 50%, 60%, 70%, 80%, or 90%. In some embodiments, tumor metastasis is prevented or slowed. In some embodiments, the tumor growth, tumor proliferation, or tumorigenicity is compared to the tumor growth, tumor proliferation, or tumorigenicity in the individual prior to the administration of the compound, salt, or composition. In some embodiments, the tumor growth, tumor proliferation, or tumorigenicity is compared to the tumor growth, tumor proliferation, or tumorigenicity in a similar individual or group of individuals. Methods of measuring tumor growth, tumor proliferation, and tumorigenicity are known in the art, for example by repeated imaging of the individual.

In accordance with the present disclosure, in some embodiments, the individual is a mammal. In some embodiments, the individual is a primate, bovine, ovine, porcine, equine, canine, feline, rabbit, or rodent. In some embodiments, the individual is a human. In some embodiments, the individual has any of the diseases or disorders disclosed herein. In some embodiments, the individual is a risk for developing any of the diseases or disorders disclosed herein.

Also provided herein are uses of a compound described herein or a pharmaceutically acceptable salt thereof, or a pharmaceutical composition described herein, in the manufacture of a medicament. In some embodiments, the manufacture of a medicament is for the treatment of a disorder or disease described herein. In some embodiments, the manufacture of a medicament is for the prevention and/or treatment of a disorder or disease mediated by SMO receptors or by the Hedgehog (Hh) signaling pathway.

In some embodiments, a compound or salt thereof described herein or a composition described herein may be used in a method as either a stand-alone therapy, or as a conjunctive therapy with other agents that are either palliative (e.g., agents that relieve the symptoms of the disorder to be treated), and/or agents that target the etiology of the disorder. Compounds or compositions of the present invention may be used or administered in combination with a second therapeutic agent. Compounds or compositions of the present invention may be used or administered in combination with an anticancer agent.

In some embodiments, (a) a compound described herein, or a pharmaceutically acceptable salt thereof, or a pharmaceutical composition described herein and (b) an agent are sequentially administered, concurrently administered or simultaneously administered. In certain embodiments, (a) a compound described herein, or a pharmaceutically acceptable salt thereof, or a pharmaceutical composition described herein and (b) an agent are administered with a time separation of about 15 minutes or less, such as about any of 10, 5, or 1 minutes or less. In certain embodiments, (a) a compound described herein, or a pharmaceutically acceptable salt thereof, or a pharmaceutical composition described herein and (b) an agent are administered with a time separation of about 15 minutes or more, such as about any of 20, 30, 40, 50, 60, or more minutes. Either (a) a compound described herein, or a pharmaceutically acceptable salt thereof, or a pharmaceutical composition described herein and (b) an agent may be administered first. In certain embodiments, (a) a compound described herein, or a pharmaceutically acceptable salt thereof, or a pharmaceutical composition described herein and (b) an agent are administered simultaneously.

### Definitions

In this application, the following definitions apply, unless indicated otherwise.

The term “SMO receptor modulator” as used herein refers to any compound which binds to and modulates the function of SMO receptors. The term “modulator” should be interpreted to include modulation by modalities including, but not limited to, antagonists and inverse agonists.

The term “treatment”, in relation to the uses of any of the compounds described herein, including those of Formula (1a) is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” (for example in relation to methods of treatment of a disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

Terms such as “phenyl”, “heterocyclic”, “alkyl”, “alkoxy”, “cycloalkyl” and “halo” are all used in their conventional sense (e.g. as defined in the IUPAC Gold Book), unless indicated otherwise. “optionally substituted” as applied to any group means that the said group may if desired be substituted with one or more substituents, which may be the same or different.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms.

The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁸O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound.

Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in some embodiments of the invention, there is provided a pharmaceutical composition comprising at least one compound of Formula (1a) as defined above together with at least one pharmaceutically acceptable excipient.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the Formula (1a) can be formulated in accordance with known techniques, see for example, Remington's Pharmaceutical Sciences, Mack Publishing Company, Easton, PA, USA. The pharmaceutical compositions can be in any form suitable for oral, parenteral, intravenous, intramuscular, intrathecal, subcutaneous, topical, intranasal, intrabronchial, sublingual, buccal, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

The composition may be a tablet composition or a capsule composition. Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments.

Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

The composition may be a parenteral composition. Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds of the Formula (1a) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### EXAMPLES

The following examples shown in Table 1 have been prepared.

TABLE 1
Examples

| | |
|---|---|
| 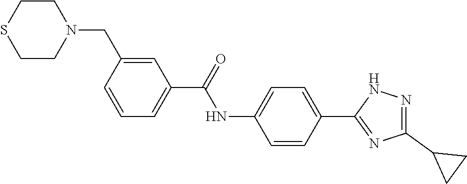 [SMILES: O=C(Nc1ccc(-c2nc(C3CC3)n[nH]2)cc1)c1cccc(CN2CCSCC2)c1] | Example 1-1 |
| 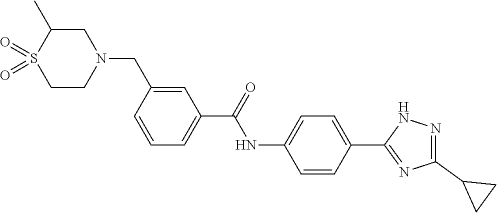 [SMILES: CC1CN(Cc2cccc(C(=O)Nc3ccc(-c4nc(C5CC5)n[nH]4)cc3)c2)CCS1(=O)=O] | Example 1-2 |
| 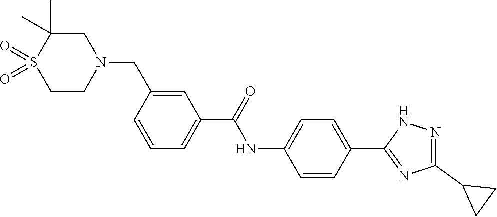 [SMILES: CC1(C)CN(Cc2cccc(C(=O)Nc3ccc(-c4nc(C5CC5)n[nH]4)cc3)c2)CCS1(=O)=O] | Example 1-3 |
| 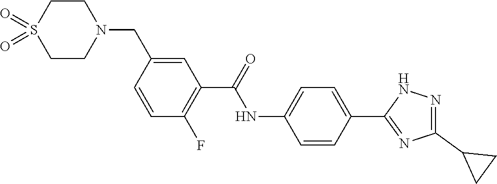 [SMILES: O=C(Nc1ccc(-c2nc(C3CC3)n[nH]2)cc1)c1cc(CN2CCS(=O)(=O)CC2)ccc1F] | Example 2-1 |
| 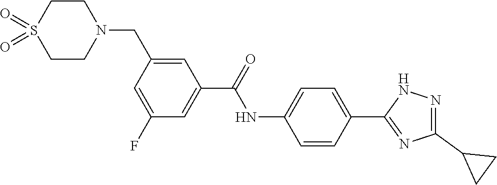 [SMILES: O=C(Nc1ccc(-c2nc(C3CC3)n[nH]2)cc1)c1cc(F)cc(CN2CCS(=O)(=O)CC2)c1] | Example 2-2 |
| 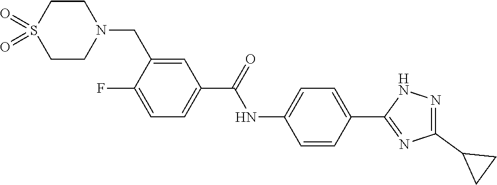 [SMILES: O=C(Nc1ccc(-c2nc(C3CC3)n[nH]2)cc1)c1ccc(F)c(CN2CCS(=O)(=O)CC2)c1] | Example 2-3 |
| 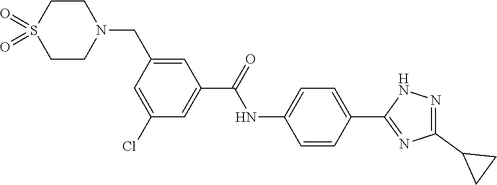 [SMILES: O=C(Nc1ccc(-c2nc(C3CC3)n[nH]2)cc1)c1cc(Cl)cc(CN2CCS(=O)(=O)CC2)c1] | Example 2-4 |
| 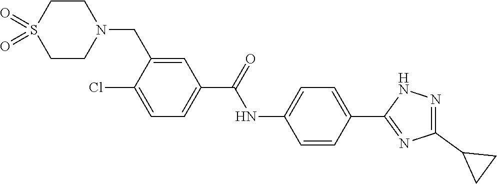 [SMILES: O=C(Nc1ccc(-c2nc(C3CC3)n[nH]2)cc1)c1ccc(Cl)c(CN2CCS(=O)(=O)CC2)c1] | Example 2-5 |
| 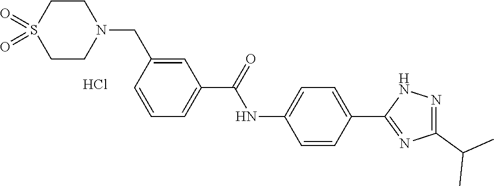 [SMILES: CC(C)c1n[nH]c(-c2ccc(NC(=O)c3cccc(CN4CCS(=O)(=O)CC4)c3)cc2)n1 ; Cl] | Example 3-1 |
| 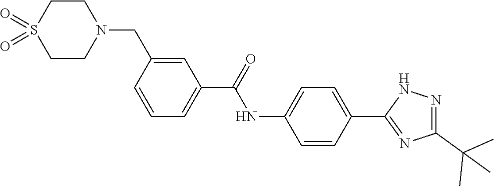 [SMILES: CC(C)(C)c1n[nH]c(-c2ccc(NC(=O)c3cccc(CN4CCS(=O)(=O)CC4)c3)cc2)n1] | Example 3-2 |
| 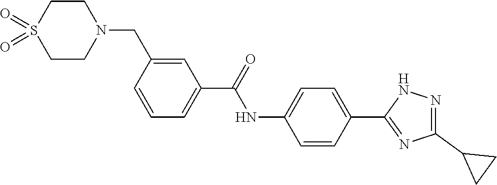 [SMILES: O=C(Nc1ccc(-c2nc(C3CC3)n[nH]2)cc1)c1cccc(CN2CCS(=O)(=O)CC2)c1] | Example 3-3 |
| 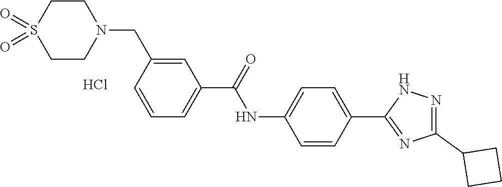 [SMILES: O=C(Nc1ccc(-c2nc(C3CCC3)n[nH]2)cc1)c1cccc(CN2CCS(=O)(=O)CC2)c1 ; Cl] | Example 3-4 |
| 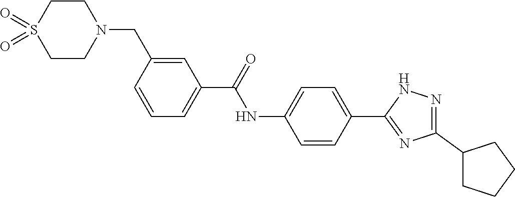 [SMILES: O=C(Nc1ccc(-c2nc(C3CCCC3)n[nH]2)cc1)c1cccc(CN2CCS(=O)(=O)CC2)c1] | Example 3-5 |
| 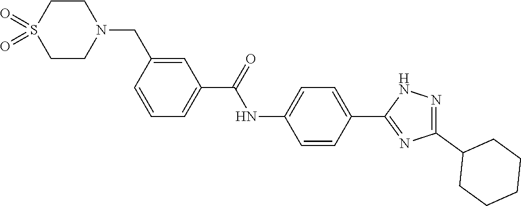 [SMILES: O=C(Nc1ccc(-c2nc(C3CCCCC3)n[nH]2)cc1)c1cccc(CN2CCS(=O)(=O)CC2)c1] | Example 3-6 |
| 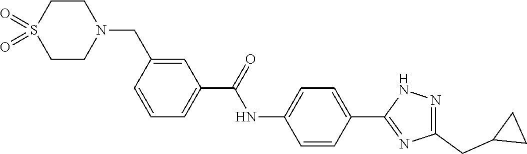 [SMILES: O=C(Nc1ccc(-c2nc(CC3CC3)n[nH]2)cc1)c1cccc(CN2CCS(=O)(=O)CC2)c1] | Example 3-7 |
| 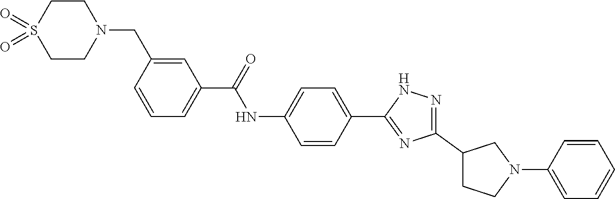 [SMILES: O=C(Nc1ccc(-c2nc(C3CCN(c4ccccc4)C3)n[nH]2)cc1)c1cccc(CN2CCS(=O)(=O)CC2)c1] | Example 3-8 |
| 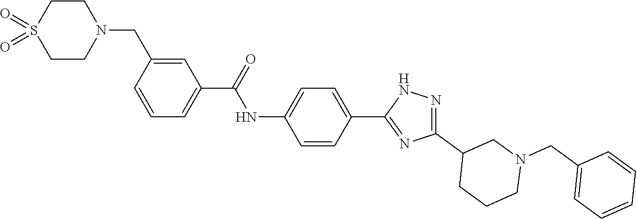 [SMILES: O=C(Nc1ccc(-c2nc(C3CCCN(Cc4ccccc4)C3)n[nH]2)cc1)c1cccc(CN2CCS(=O)(=O)CC2)c1] | Example 3-9 |
| 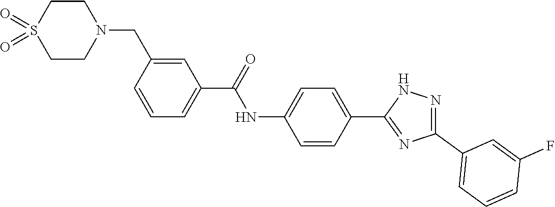 [SMILES: O=C(Nc1ccc(-c2nc(-c3cccc(F)c3)n[nH]2)cc1)c1cccc(CN2CCS(=O)(=O)CC2)c1] | Example 3-10 |
| 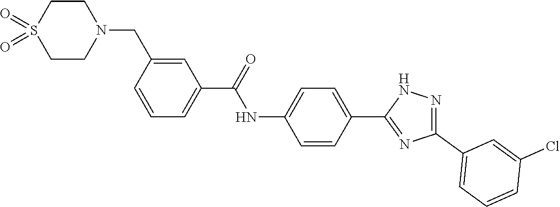 [SMILES: O=C(Nc1ccc(-c2nc(-c3cccc(Cl)c3)n[nH]2)cc1)c1cccc(CN2CCS(=O)(=O)CC2)c1] | Example 3-11 |
| 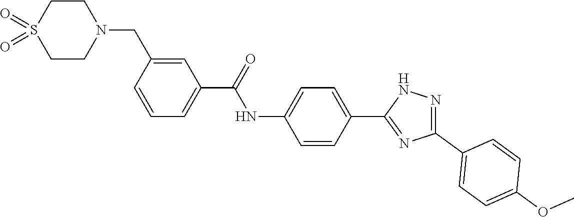 [SMILES: COc1ccc(-c2n[nH]c(-c3ccc(NC(=O)c4cccc(CN5CCS(=O)(=O)CC5)c4)cc3)n2)cc1] | Example 3-12 |
| 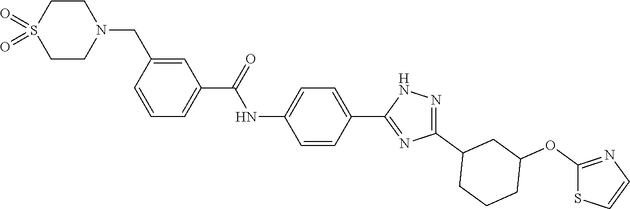 [SMILES: O=C(Nc1ccc(-c2nc(C3CCCC(Oc4nccs4)C3)n[nH]2)cc1)c1cccc(CN2CCS(=O)(=O)CC2)c1] | Example 3-13 |
| 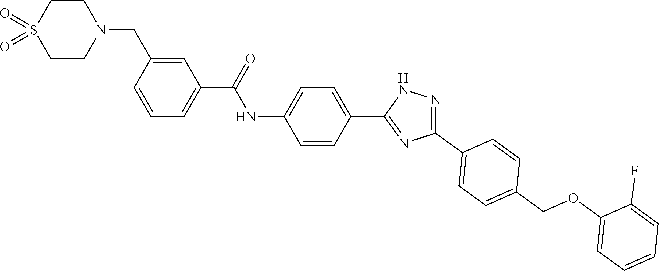 [SMILES: O=C(Nc1ccc(-c2nc(-c3ccc(COc4ccccc4F)cc3)n[nH]2)cc1)c1cccc(CN2CCS(=O)(=O)CC2)c1] | Example 3-14 |
| 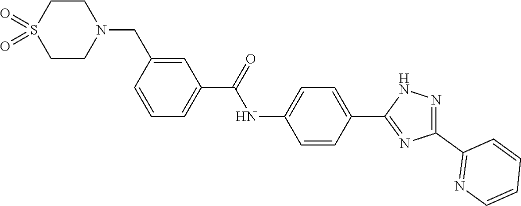 [SMILES: O=C(Nc1ccc(-c2nc(-c3ccccn3)n[nH]2)cc1)c1cccc(CN2CCS(=O)(=O)CC2)c1] | Example 3-15 |
| 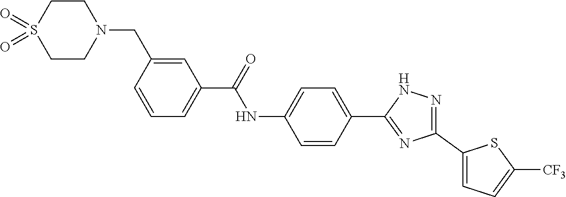 [SMILES: O=C(Nc1ccc(-c2nc(-c3ccc(C(F)(F)F)s3)n[nH]2)cc1)c1cccc(CN2CCS(=O)(=O)CC2)c1] | Example 3-16 |
| 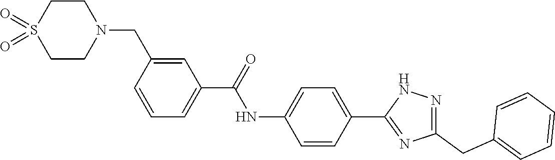 [SMILES: O=C(Nc1ccc(-c2nc(Cc3ccccc3)n[nH]2)cc1)c1cccc(CN2CCS(=O)(=O)CC2)c1] | Example 3-17 |
| 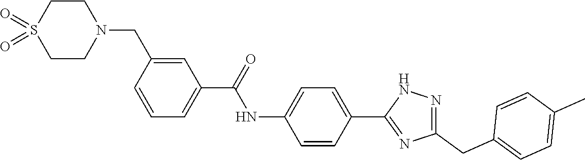 [SMILES: Cc1ccc(Cc2n[nH]c(-c3ccc(NC(=O)c4cccc(CN5CCS(=O)(=O)CC5)c4)cc3)n2)cc1] | Example 3-18 |
| 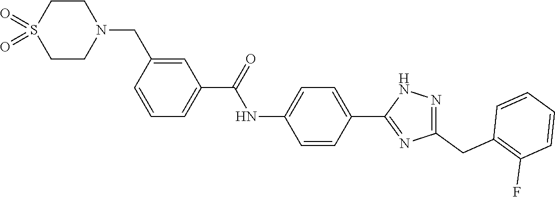 [SMILES: O=C(Nc1ccc(-c2nc(Cc3ccccc3F)n[nH]2)cc1)c1cccc(CN2CCS(=O)(=O)CC2)c1] | Example 3-19 |
| 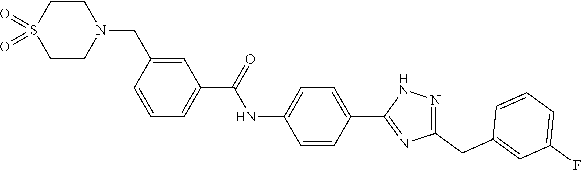 [SMILES: O=C(Nc1ccc(-c2nc(Cc3cccc(F)c3)n[nH]2)cc1)c1cccc(CN2CCS(=O)(=O)CC2)c1] | Example 3-20 |
| 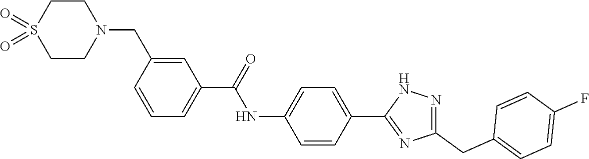 [SMILES: O=C(Nc1ccc(-c2nc(Cc3ccc(F)cc3)n[nH]2)cc1)c1cccc(CN2CCS(=O)(=O)CC2)c1] | Example 3-21 |
| 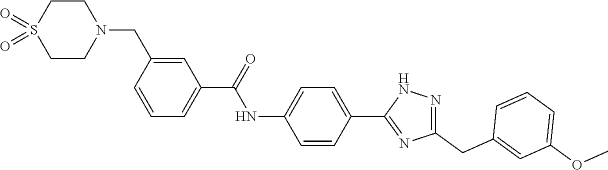 [SMILES: COc1cccc(Cc2n[nH]c(-c3ccc(NC(=O)c4cccc(CN5CCS(=O)(=O)CC5)c4)cc3)n2)c1] | Example 3-22 |
| 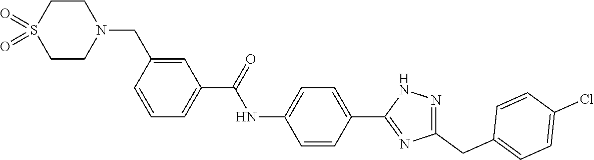 [SMILES: O=C(Nc1ccc(-c2nc(Cc3ccc(Cl)cc3)n[nH]2)cc1)c1cccc(CN2CCS(=O)(=O)CC2)c1] | Example 3-23 |
| 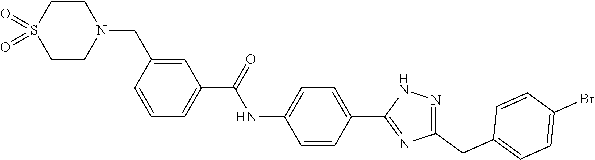 [SMILES: O=C(Nc1ccc(-c2nc(Cc3ccc(Br)cc3)n[nH]2)cc1)c1cccc(CN2CCS(=O)(=O)CC2)c1] | Example 3-24 |
| 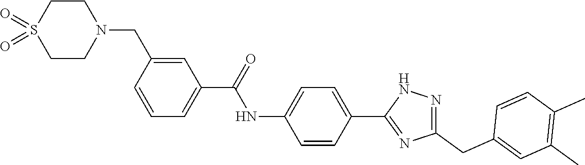 [SMILES: Cc1ccc(Cc2n[nH]c(-c3ccc(NC(=O)c4cccc(CN5CCS(=O)(=O)CC5)c4)cc3)n2)cc1C] | Example 3-25 |
| 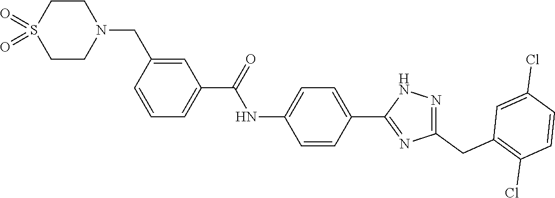 [SMILES: O=C(Nc1ccc(-c2nc(Cc3cc(Cl)ccc3Cl)n[nH]2)cc1)c1cccc(CN2CCS(=O)(=O)CC2)c1] | Example 3-26 |
| 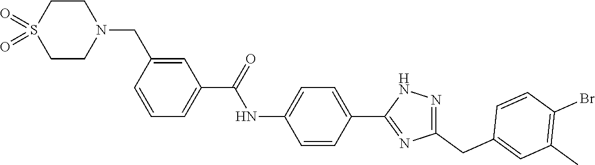 [SMILES: Cc1cc(Cc2n[nH]c(-c3ccc(NC(=O)c4cccc(CN5CCS(=O)(=O)CC5)c4)cc3)n2)ccc1Br] | Example 3-27 |
| 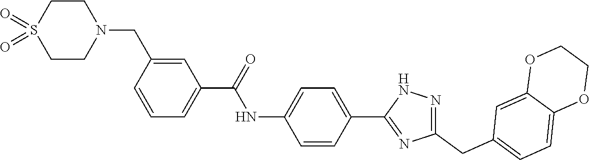 [SMILES: O=C(Nc1ccc(-c2nc(Cc3ccc4c(c3)OCCO4)n[nH]2)cc1)c1cccc(CN2CCS(=O)(=O)CC2)c1] | Example 3-28 |
| 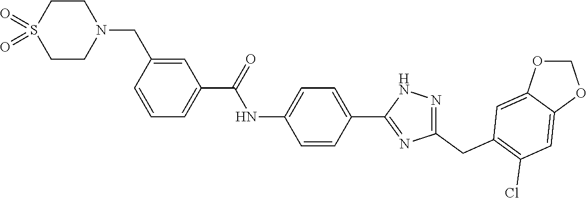 [SMILES: O=C(Nc1ccc(-c2nc(Cc3cc4c(cc3Cl)OCO4)n[nH]2)cc1)c1cccc(CN2CCS(=O)(=O)CC2)c1] | Example 3-29 |
| 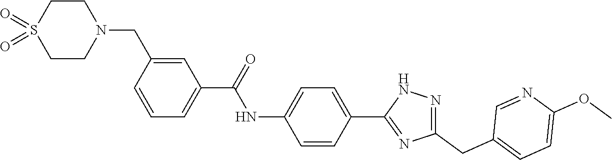 [SMILES: COc1ccc(Cc2n[nH]c(-c3ccc(NC(=O)c4cccc(CN5CCS(=O)(=O)CC5)c4)cc3)n2)cn1] | Example 3-30 |
| 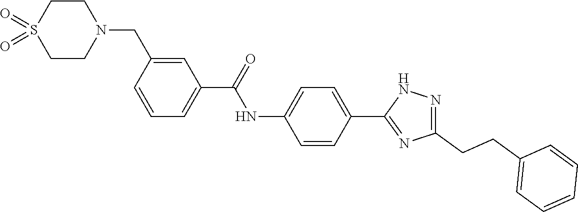 [SMILES: O=C(Nc1ccc(-c2nc(CCc3ccccc3)n[nH]2)cc1)c1cccc(CN2CCS(=O)(=O)CC2)c1] | Example 3-31 |
| 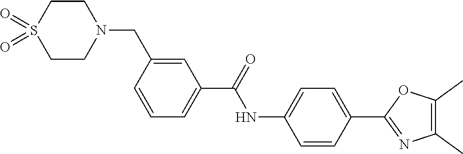 [SMILES: Cc1nc(-c2ccc(NC(=O)c3cccc(CN4CCS(=O)(=O)CC4)c3)cc2)oc1C] | Example 4-1 |
| 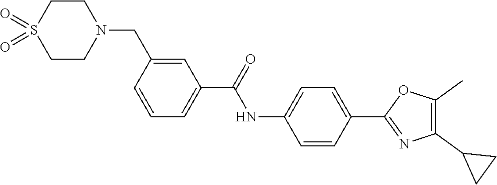 [SMILES: Cc1oc(-c2ccc(NC(=O)c3cccc(CN4CCS(=O)(=O)CC4)c3)cc2)nc1C1CC1] | Example 4-2 |
| 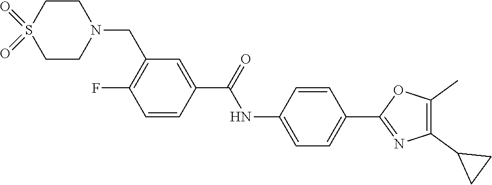 [SMILES: Cc1oc(-c2ccc(NC(=O)c3ccc(F)c(CN4CCS(=O)(=O)CC4)c3)cc2)nc1C1CC1] | Example 4-3 |
| 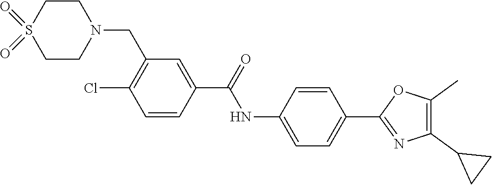 [SMILES: Cc1oc(-c2ccc(NC(=O)c3ccc(Cl)c(CN4CCS(=O)(=O)CC4)c3)cc2)nc1C1CC1] | Example 4-4 |
| 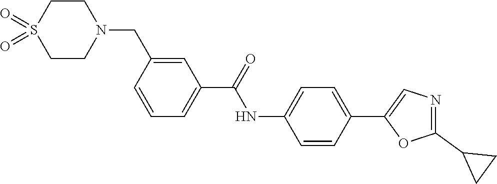 [SMILES: O=C(Nc1ccc(-c2cnc(C3CC3)o2)cc1)c1cccc(CN2CCS(=O)(=O)CC2)c1] | Example 4-5 |
| 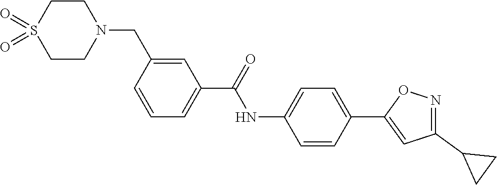 [SMILES: O=C(Nc1ccc(-c2cc(C3CC3)no2)cc1)c1cccc(CN2CCS(=O)(=O)CC2)c1] | Example 4-6 |
| 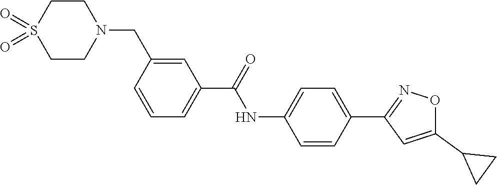 [SMILES: O=C(Nc1ccc(-c2cc(C3CC3)on2)cc1)c1cccc(CN2CCS(=O)(=O)CC2)c1] | Example 4-7 |
| 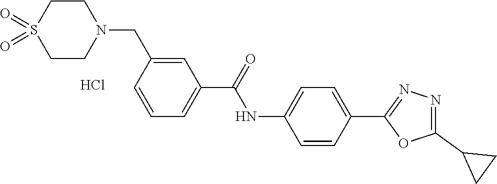 [SMILES: O=C(Nc1ccc(-c2nnc(C3CC3)o2)cc1)c1cccc(CN2CCS(=O)(=O)CC2)c1 ; Cl] | Example 4-8 |
| 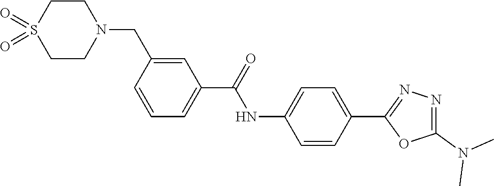 [SMILES: CN(C)c1nnc(-c2ccc(NC(=O)c3cccc(CN4CCS(=O)(=O)CC4)c3)cc2)o1] | Example 4-9 |
| 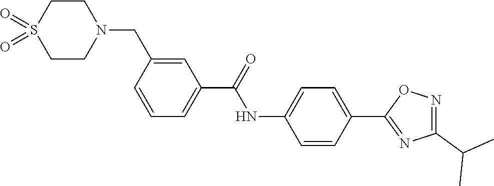 [SMILES: CC(C)c1noc(-c2ccc(NC(=O)c3cccc(CN4CCS(=O)(=O)CC4)c3)cc2)n1] | Example 4-10 |
| 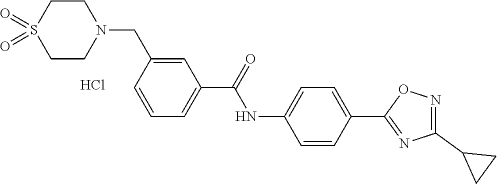 [SMILES: O=C(Nc1ccc(-c2nc(C3CC3)no2)cc1)c1cccc(CN2CCS(=O)(=O)CC2)c1 ; Cl] | Example 4-11 |
| 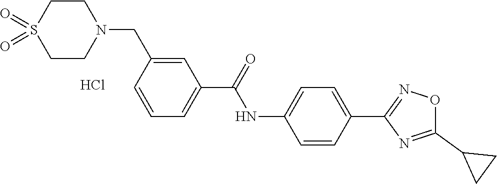 [SMILES: O=C(Nc1ccc(-c2noc(C3CC3)n2)cc1)c1cccc(CN2CCS(=O)(=O)CC2)c1 ; Cl] | Example 4-12 |
| 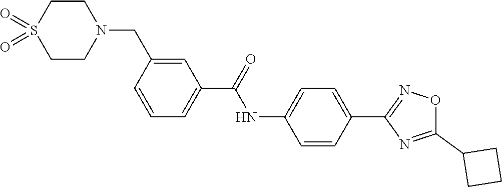 [SMILES: O=C(Nc1ccc(-c2noc(C3CCC3)n2)cc1)c1cccc(CN2CCS(=O)(=O)CC2)c1] | Example 4-13 |
| 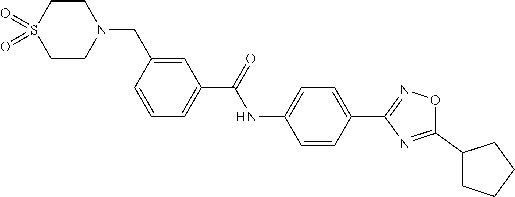 [SMILES: O=C(Nc1ccc(-c2noc(C3CCCC3)n2)cc1)c1cccc(CN2CCS(=O)(=O)CC2)c1] | Example 4-14 |
| 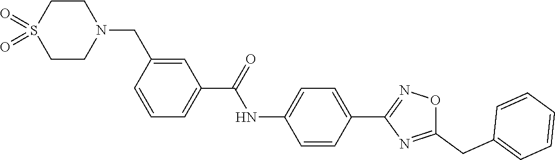 [SMILES: O=C(Nc1ccc(-c2noc(Cc3ccccc3)n2)cc1)c1cccc(CN2CCS(=O)(=O)CC2)c1] | Example 4-15 |
| 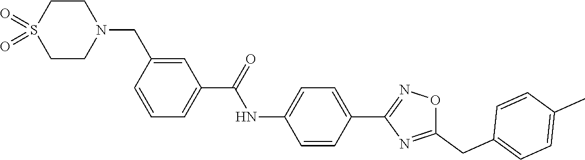 [SMILES: Cc1ccc(Cc2nc(-c3ccc(NC(=O)c4cccc(CN5CCS(=O)(=O)CC5)c4)cc3)no2)cc1] | Example 4-16 |
| 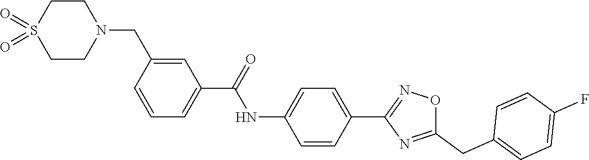 [SMILES: O=C(Nc1ccc(-c2noc(Cc3ccc(F)cc3)n2)cc1)c1cccc(CN2CCS(=O)(=O)CC2)c1] | Example 4-17 |
| 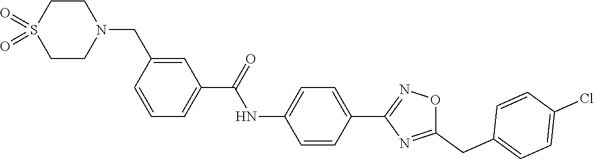 [SMILES: O=C(Nc1ccc(-c2noc(Cc3ccc(Cl)cc3)n2)cc1)c1cccc(CN2CCS(=O)(=O)CC2)c1] | Example 4-18 |
| 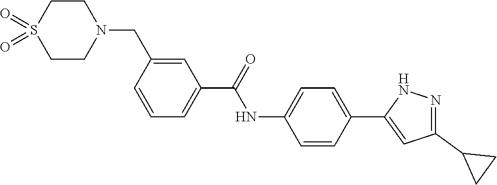 [SMILES: O=C(Nc1ccc(-c2cc(C3CC3)n[nH]2)cc1)c1cccc(CN2CCS(=O)(=O)CC2)c1] | Example 4-19 |
| 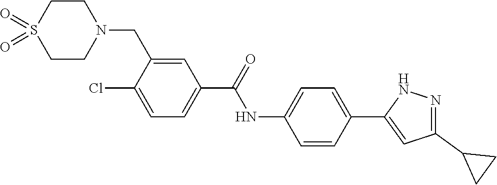 [SMILES: O=C(Nc1ccc(-c2cc(C3CC3)n[nH]2)cc1)c1ccc(Cl)c(CN2CCS(=O)(=O)CC2)c1] | Example 4-20 |
| 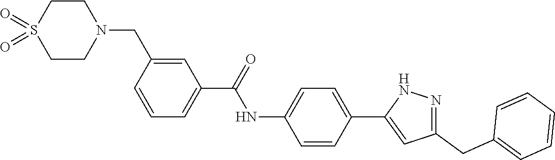 [SMILES: O=C(Nc1ccc(-c2cc(Cc3ccccc3)n[nH]2)cc1)c1cccc(CN2CCS(=O)(=O)CC2)c1] | Example 4-21 |
| 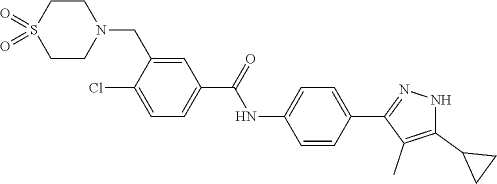 [SMILES: Cc1c(-c2ccc(NC(=O)c3ccc(Cl)c(CN4CCS(=O)(=O)CC4)c3)cc2)n[nH]c1C1CC1] | Example 4-22 |
| 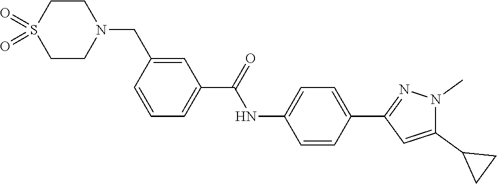 [SMILES: Cn1nc(-c2ccc(NC(=O)c3cccc(CN4CCS(=O)(=O)CC4)c3)cc2)cc1C1CC1] | Example 4-23 |
| 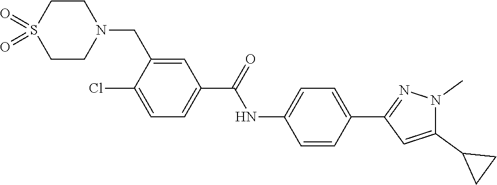 [SMILES: Cn1nc(-c2ccc(NC(=O)c3ccc(Cl)c(CN4CCS(=O)(=O)CC4)c3)cc2)cc1C1CC1] | Example 4-24 |
| 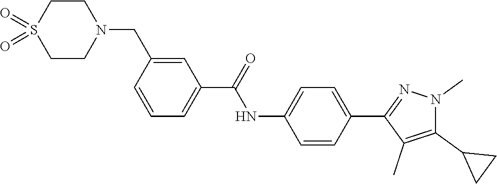 [SMILES: Cc1c(-c2ccc(NC(=O)c3cccc(CN4CCS(=O)(=O)CC4)c3)cc2)nn(C)c1C1CC1] | Example 4-25 |
| 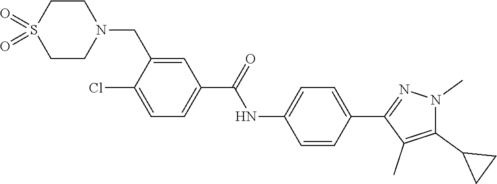 [SMILES: Cc1c(-c2ccc(NC(=O)c3ccc(Cl)c(CN4CCS(=O)(=O)CC4)c3)cc2)nn(C)c1C1CC1] | Example 4-26 |
| 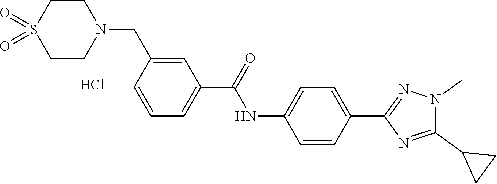 [SMILES: Cn1nc(-c2ccc(NC(=O)c3cccc(CN4CCS(=O)(=O)CC4)c3)cc2)nc1C1CC1 ; Cl] | Example 4-27 |
| 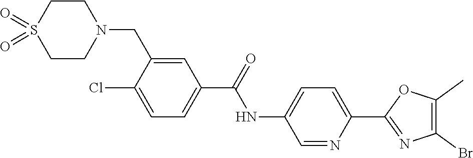 [SMILES: Cc1oc(-c2ccc(NC(=O)c3ccc(Cl)c(CN4CCS(=O)(=O)CC4)c3)cn2)nc1Br] | Example -51 |
| 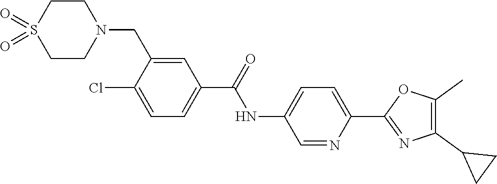 [SMILES: Cc1oc(-c2ccc(NC(=O)c3ccc(Cl)c(CN4CCS(=O)(=O)CC4)c3)cn2)nc1C1CC1] | Example 5-2 |
| 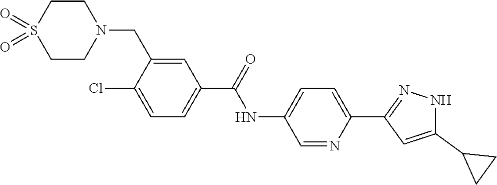 [SMILES: O=C(Nc1ccc(-c2cc(C3CC3)[nH]n2)nc1)c1ccc(Cl)c(CN2CCS(=O)(=O)CC2)c1] | Example 5-3 |
| 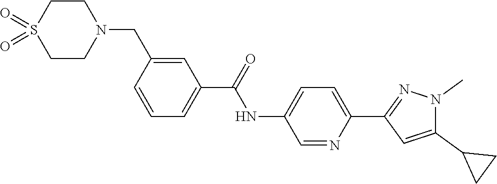 [SMILES: Cn1nc(-c2ccc(NC(=O)c3cccc(CN4CCS(=O)(=O)CC4)c3)cn2)cc1C1CC1] | Example 5-4 |
| 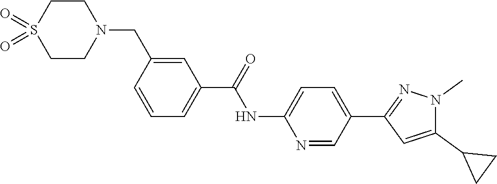 [SMILES: Cn1nc(-c2ccc(NC(=O)c3cccc(CN4CCS(=O)(=O)CC4)c3)nc2)cc1C1CC1] | Example 5-5 |

### Preparation of the Compounds of the Invention

Some compounds of Formula (1a) and derivatives or synthetic intermediates thereof can be prepared in accordance with synthetic methods known to the skilled person. In some embodiments, the invention provides a process for the preparation of a compound as defined in Formula (1a) above. Compounds of the invention may be prepared according to the methods described below.

### Abbreviations

AcOH=acetic acid
CDl=1,1′-Carbonyldiimidazole
d=day(s)
DAST=diethylaminosulfur trifluoride
DCE=dichloroethane
DCM=dichloromethane
DIPEA=diisopropylethylamine
DIAD=diisopropyl azodicarboxylate
DMAP=4-dimethylaminopyridine
DMF=dimethytformamide
DMP=Dess-Martin periodinane
DMSO=dimethylsulfoxide
EDCl=N-ethyl-N-(3-dimethylaminopropyl)carbodiimide hydrochloride
ESI=electro spray ionisation
EtOAc=ethyl acetate
EtOH=ethanol
h=hour(s)
HATU=1-[Bis(dimethylamino)methylene]-1H-1,2,3-triazolo[4,5-b]pyridinium 3-oxid hexafluorophosphate
HOBt=1-hydroxybenzotriazole
HPLC=high performance liquid chromatography
LC=liquid chromatography
LiAlH₄/LAH=Lithium aluminium hydride
MeCN=acetonitrile
MeOH=methanol
min=minute(s)
MS=mass spectrometry
NBS=N-bromosuccinimide
NCS=N-chlorosuccinimide
Et₃N=triethylamine
NMR=nuclear magnetic resonance
R=retention time
RT=room temperature
sat.=saturated
sol.=solution
STAB=sodium triacetoxyborohydride
TFA=trifluoroacetic acid
THF=tetrahydrofuran
TLC=thin layer chromatography

Prefixes n-, s-, i-, t- and tert- have their usual meanings: normal, secondary, iso, and tertiary.

### LCMS Analysis

LCMS analysis of compounds was performed under electrospray conditions using the instruments and methods given below:

| System | Instrument Name | LC Detector | Mass Detector |
|---|---|---|---|
| 1 | Agilent Technologies 1290 | Photo Diode | 6125 Quadrupole |
| | Infinity II Series LC | Array | MSD SL |
| 2 | Shimadzu LC-20AD | Photo Diode | Shimadzu MS |
| | | Array | 2010 |
| 3 | Agilent 1200 | Photo Diode | Agilent 6110B |
| | | Array | |
| 4 | Agilent Technologies 1290 | Photo Diode | 6130 Quadrupole |
| | Infinity II Series LC | Array | MSD |
| 5 | Agilent 1200 | Photo Diode | Agilent 6120B |
| | | Array/ELSD | |

TABLE 2
Table 2 - LCMS analysis methods

| | | | | | | Flow |
|---|---|---|---|---|---|---|
| Method | Solvent | Column | | UV | Column | Rate |
| Name | System | used | Gradient | Range | Temp. ° C. | ml/min |
| A | (A) 0.1% TFA in | XBridgeC8 3.5μ, | 95:5 at 0.0 min, | 210-400 | 25 | 1.5 |
| | water | 4.6 × 50 mm | 5:95 at 2.5 min, | nm | | |
| | (B) 0.1% TFA in | | 5:95 at 4.0 min, | | | |
| | MeCN | | 95:5 at 4.5 min, | | | |
| | | | 95:5 at 6.0 min | | | |
| B | (A) 0.1% formic | Zorbax eclipse | 95:5 at 0.0 min, | 210-400 | 25 | 0.8 |
| | acid in | plus C18 1.8μ, | 95:5 at 0.25 min, | nm | | |
| | water:MeCN | 2.1 × 50 mm | 0:100 at 2.5 min, | | | |
| | (95:5) | | 0:100 at 3.0 min, | | | |
| | (B) MeCN | | 95:5 at 3.1 min, | | | |
| | | | 95:5 at 4.0 min | | | |
| C | (A) 0.1% formic | Zorbax XDB | 95:5 at 0.0 min, | 210-400 | 25 | 1.5 |
| | acid in | C18 5μ, | 5:95 at 2.5 min, | nm | | |
| | water:MeCN | 4.6 × 50 mm | 5:95 at 4.0 min, | | | |
| | (95:5) | | 95:5 at 4.5 min, | | | |
| | (B) MeCN | | 95:5 at 6.0 min | | | |
| D | (A) 0.1% formic | Atlantis dC18 | 95:5 at 0.0 min, | 210-400 | 25 | 1.5 |
| | acid in | 5μ, | 5:95 at 2.5 min, | nm | | |
| | water:MeCN | 4.6 × 50 mm | 5:95 at 4.0 min, | | | |
| | (95:5) | | 95:5 at 4.5 min, | | | |
| | (B) MeCN | | 95:5 at 6.0 min | | | |
| E | (A) 0.037% TFA | Luna C18 3μ, | 90:10 at 0.01 min, | 200-400 | Ambient | 0.8 |
| | in water | 2.0 × 30 mm | 20:80 at 1.15 min, | nm | | (0.01-1.51 |
| | (B) 0.018% TFA | | 20:80 at 1.65 min, | | | min), |
| | in MeCN | | 90:10 at 1.66 min, | | | 1.2 |
| | | | 90:10 at 2.00 min, | | | (1.52-2.00 |
| | | | | | | min) |
| F | (A) 0.1% formic | Zorbax extend | 95:5 at 0.0 min, | 210-400 | 25 | 1.5 |
| | acid in | C18 5μ, | 5:95 at 2.5 min, | nm | | |
| | water:MeCN | 4.6 × 50 mm | 5:95 at 4.0 min, | | | |
| | (95:5) | | 95:5 at 4.5 min, | | | |
| | (B) MeCN | | 95:5 at 5.5 min | | | |
| G | (A) 10 mM | Acquity BEH | 95:5 at 0.0 min, | 210-400 | 25 | 1.5 |
| | ammonium | C18 1.7μ, | 95:5 at 0.25 min, | nm | | |
| | acetate | 2.1 × 50 mm | 0:100 at 2.5 min, | | | |
| | in water | | 0:100 at 3.0 min, | | | |
| | (B) MeCN | | 95:5 at 3.1 min, | | | |
| | | | 95:5 at 4.0 min | | | |
| H | (A) 10 mM | XBridgeC8 | 90:10 at 0.0 min, | 210-400 | 25 | 1.2 |
| | ammonium | 3.5μ, | 5:95 at 4.0 min, | nm | | |
| | bicarbonate | 4.6 × 50 mm | 5:95 at 5.0 min, | | | |
| | in water | | 90:10 at 5.5 min, | | | |
| | (B) MeCN | | 90:10 at 7.0 min | | | |
| I | (A) 10 mM | Xbridge Shield | 95:5 at 0.01 min, | 200-400 | Ambient | 1.0 |
| | ammonium | RP18 5μ, | 5:95 at 1.00 min, | nm | | |
| | bicarbonate | 2.1 × 50 mm | 0:100 at 1.80 min, | | | |
| | in water | | 95:5 at 1.81 min, | | | |
| | (B) MeCN | | 95:5 at 2.20 min, | | | |
| J | (A) 0.037% TFA | Kinetex | 95:5 at 0.01 min, | 200-400 | Ambient | 1.0 |
| | in water | C18 5μ, | 5:95 at 0.40 min, | nm | | |
| | (B) 0.018% TFA | 50 × 2.1 mm | 5:95 at 3.00 min, | | | |
| | in MeCN | | 5:95 at 4.00 min, | | | |
| | | | 95:5 at 4.01 min, | | | |
| | | | 95:5 at 5.00 min | | | |
| K | (A) 10 mM | Zorbax extend | 90:10 at 0.0 min, | 210-400 | 25 | 1.5 |
| | ammonium | C18 5μ, | 5:95 at 4.0 min, | nm | | |
| | acetate | 4.6 × 50 mm | 5:95 at 5.0 min, | | | |
| | in water | | 90:10 at 4.5 min, | | | |
| | (B) MeCN | | 95:5 at 5.5 min | | | |
| L | (A) 10 mM | Zorbax XDB | 90:10 at 0.0 min, | 210-400 | 25 | 1.2 |
| | ammonium | C18 5μ, | 5:95 at 3.5 min, | nm | | |
| | acetate | 4.6 × 50 mm | 5:95 at 4.5 min, | | | |
| | in water | | 90:10 at 5 min, | | | |
| | (B) MeCN | | 90:10 at 5.5 min | | | |
| M | (A) 0.1% formic | Poroshell 120 | 99:1 at 0.01 min, | 215-280 | 60 | 3.0 |
| | acid in water | SB-C18 2.7μ, | 0:100 at 1.50 min, | nm | | |
| | (B) 0.1% formic | 4.6 × 30 mm | 0:100 at 2.20 min, | | | |
| | acid in MeCN | | | | | |
| N | (A) 0.1% formic | Poroshell 120 | 99:1 at 0.01 min, | 215-280 | 60 | 1.5 |
| | acid in water | SB-C18 2.7μ, | 0:100 at 5.00 min, | nm | | |
| | (B) 0.1% formic | 4.6 × 30 mm | 0:100 at 5.99 min, | | | |
| | acid in MeCN | | | | | |

LCMS data in the experimental section and Tables 3 and 4 are given in the format: (instrument system, Method): Mass ion, retention time, UV detection.

### Compound Purification

Final purification of compounds was performed by filtration, flash column chromatography or preparative reversed phase HPLC using the instruments and methods detailed below where data is given in the following format: Purification technique: [phase (column description, column length×internal diameter, particle size), solvent flow-rate, gradient—given as % of mobile phase B in mobile phase A (over time), mobile phase (A), mobile phase (B)].

### Flash Column Chromatography

Biotage-Isolera

### Preparative HPLC Purification:

Agilent Technologies 1260 Infinity II Series LC

Shimadzu LC-20AP binary system with SPD-20A UV detector

### Purification Method A

Flash column chromatography: (silica, eluting with Pet-ether:EtOAc).

### Purification Method B

Reaction mixture was poured onto ice and the resulting solid collected by filtration and dried.

### Purification Method C

Prep HPLC: [Reverse Phase (XBridge C8, 150×19 mm, 5 μm) gradient 10%-95% (over 15 min), 95% (over 3 min), 95%-10% (over 1 min), 10% (over 2 min), mobile phase (A): 10 mM ammonium bicarbonate in water, (B): 100% acetonitrile].

### Purification Method D

Prep HPLC: [Reverse Phase (Welch Xtimate C18, 150×25 mm, 5 μm), 15 mL/min, gradient 10%-40% (over 8 min), mobile phase (A): 0.04% HCl in water, (B): 100% acetonitrile].

### Purification Method E

Prep HPLC: [Reverse Phase (Sunfire C18, 150×19 mm, 5 μm) gradient 10%-95% (over 20 min), 95% (over 3 min), 95%-10% (over 1 min), 10% (over 2 min), mobile phase (A): 0.1% TFA in water, (B): 100% acetonitrile]. The fraction containing the desired compound was concentrated and neutralised with saturated aq. NaHCO₃.

### Purification Method F

Prep HPLC: [Reverse Phase (Sunfire C18, 150×30 mm, 5 μm) gradient 10%-95% (over 20 min), 95% (over 3 min), 95%-10% (over 1 min), 10% (over 2 min), mobile phase (A): 0.1% Formic acid in water, (B): 100% acetonitrile]. The fraction containing the desired compound was concentrated and neutralised with saturated aq. NaHCO₃.

### Purification Method G

Prep HPLC: [Reverse Phase (XBridge C18, 150×19 mm, 5 μm) gradient 10%-95% (over 15 min), 95% (over 3 min), 95%-10% (over 1 min), 10% (over 2 min), mobile phase (A): 10 mM ammonium acetate in water, (B): 100% acetonitrile]. The fraction containing the desired compound was concentrated and neutralised with saturated aq. NaHCO₃.

### Purification Method H

Prep HPLC: [Reverse Phase (XBridge C18, 150×40 mm, 10 μm), gradient 35%-65% (over 8 min), mobile phase (A): 10 mM ammonium bicarbonate in water, (B): 100% acetonitrile].

### Purification Method I

Prep HPLC: [Reverse Phase (XBridge C18, 150×40 mm, 10 μm), gradient 45%-75% (over 8 min), mobile phase (A): 10 mM ammonium bicarbonate in water, (B): 100% acetonitrile].

### Purification Method J

Prep HPLC: [Reverse Phase (XBridge C18, 150×40 mm, 10 μm), gradient 40%-70% (over 8 min), mobile phase (A): 10 mM ammonium bicarbonate in water, (B): 100% acetonitrile].

### Purification Method K

Prep HPLC: [Reverse Phase (XBridge BEH C18, 100×25 mm, 5 μm), gradient 45%-75% (over 10 min), mobile phase (A): 10 mM ammonium bicarbonate in water, (B): 100% acetonitrile].

### Purification Method L

Prep HPLC: [Reverse Phase (Luna C18, 250×100 mm, 10 μm), gradient 30%-60% (over 20 min), mobile phase (A): 0.05% HCl in water, (B): 100% acetonitrile].

### Purification Method M

Prep HPLC: [Reverse Phase (Welch Xtimate C18, 150×25 mm, 5 μm), 15 mL/min, gradient 1%-30% (over 8 min), mobile phase (A): 0.04% HCl in water, (B): 100% acetonitrile].

### Synthesis of Intermediates:

### Route 1

### Typical Procedure for the Preparation of Benzolic Acids, as Exemplified by the Preparation of Intermediate 3,3-(thiomorpholinomethyl)benzoic acid

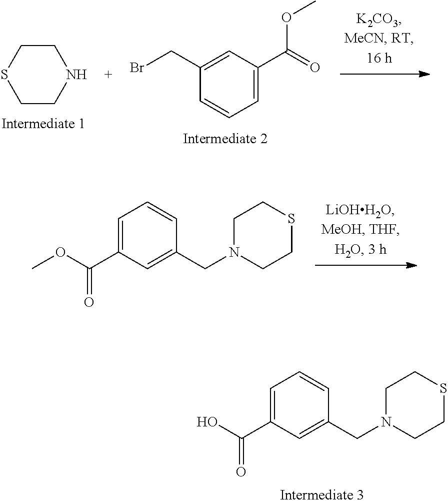 [SMILES: C1CSCCN1 ; COC(=O)c1cccc(CBr)c1 ; COC(=O)c1cccc(CN2CCSCC2)c1 ; O=C(O)c1cccc(CN2CCSCC2)c1]

To a stirred solution of Intermediate 1 (250 mg, 2.42 mmol) in MeCN (5 mL) were added K₂CO₃ (402 mg, 2.91 mmol) and Intermediate 2 (555 mg, 1.09 mmol). The reaction mixture was stirred at RT for 16 h. After the consumption of the starting material the reaction mixture was filtered through celite, the filtrate was evaporated. The residue was purified by flash column chromatography (25 g silica, eluted with Pet-ether:EtOAc (90:10 to 65:35)). The desired fractions were combined and concentrated under reduced pressure to afford methyl 3-((2-methyl-1,1-dioxidothiomorpholino)methyl)benzoate (525 mg, 86%) as colourless liquid.

LCMS (System 1, Method A): m/z 252 (M+H)⁺ (ESI⁺), Rₜ 0.54 min, 99.6%, UV active.

To a stirred solution of methyl 3-((2-methyl-1,1-dioxidothiomorpholino)methyl)benzoate (525 mg, 2.08 mmol) in THF:MeOH:water (7:2:1) (10 mL) was added LiOH·H₂O (131 mg, 3.13 mmol). The reaction mass was stirred at RT for 3 h. After the consumption of the starting material the reaction mixture was evaporated. The crude product was acidified with citric acid, the solid obtained was filtered, washed with water and dried to afford Intermediate 3 (275 mg, 55%) as a white solid.

The data for Intermediate 3 are in Table 3.

### Route 2

### Typical Procedure for the Preparation of N-Oxides, as Exemplified by the Preparation of Intermediate 9, 4-(3-((4-(3-cyclopropyl-1H-1,2,4-triazol-5-yl)phenyl)carbamoyl)benzyl)-2,2-dimethylthiomorpholine 4-oxide 1,1-dioxide

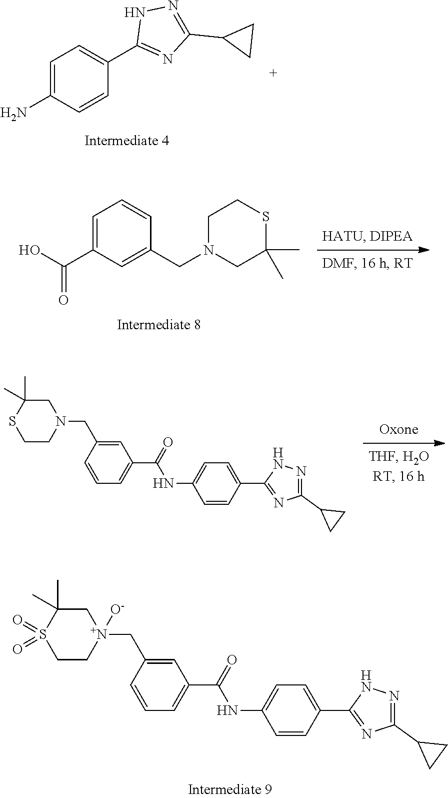 [SMILES: Nc1ccc(-c2nc(C3CC3)n[nH]2)cc1 ; CC1(C)CN(Cc2cccc(C(=O)O)c2)CCS1 ; CC1(C)CN(Cc2cccc(C(=O)Nc3ccc(-c4nc(C5CC5)n[nH]4)cc3)c2)CCS1 ; CC1(C)C[N+]([O-])(Cc2cccc(C(=O)Nc3ccc(-c4nc(C5CC5)n[nH]4)cc3)c2)CCS1(=O)=O]

To a stirred solution of Intermediate 8 (450 mg, 1.69 mmol) in DMF (10 mL) at 0° C. were added DIPEA (0.88 mL, 5.09 mmol), HATU (658 mg, 2.54 mmol) and Intermediate 4 (340 mg, 1.696 mmol). The reaction mixture was stirred at RT for 16 h. After the consumption of the starting material the reaction mixture was diluted with water (10 mL) and extracted with EtOAc (2×15 mL). The combined organic layers were washed with brine solution (15 mL), dried over anhydrous Na₂SO₄ and concentrated under reduced pressure. The residue was purified by flash column chromatography (25 g silica, eluted with Pet-ether:EtOAc (50:50 to 40:60)). The desired fractions were combined and concentrated under reduced pressure to afford N-(4-(3-cyclopropyl-1H-1,2,4-triazol-5-yl)phenyl)-3-((2,2-dimethylthiomorpholino)methyl)benzamide (200 mg, 26%) as a brown solid.

LCMS (System 1, Method C): m/z 448 (M+H)⁺ (ESI⁺), Rₜ 1.097 min, 60.9%, UV active.

To a stirred solution of N-(4-(3-cyclopropyl-1H-1,2,4-triazol-5-yl)phenyl)-3-((2,2-dimethylthiomorpholino)methyl)benzamide (150 mg, 0.335 mmol) in THF:water (7:3) (10 mL) was added Oxone (257 mg, 0.838 mmol). The resultant reaction mixture was stirred at RT for 16 h. After the consumption of the starting material the solvent was concentrated under reduced pressure. A 125 mg portion of the crude material was used for next step and the remaining portion was purified by Purification Method C. The desired fractions were combined and concentrated under reduced pressure. The product was dissolved in DCM (5 mL), washed with water (5 mL), dried over Na₂SO₄, evaporated and lyophilized to afford Intermediate 9 (10 mg, 6%) as an off white solid.

The data for Intermediate 9 are in Table 3.

### Route 3

### Typical Procedure for the Preparation of Triazole Anilines, as Exemplified by the Preparation of Intermediate 15, 4-(3-cyclobutyl-1H-1,2,4-triazol-5-yl)aniline

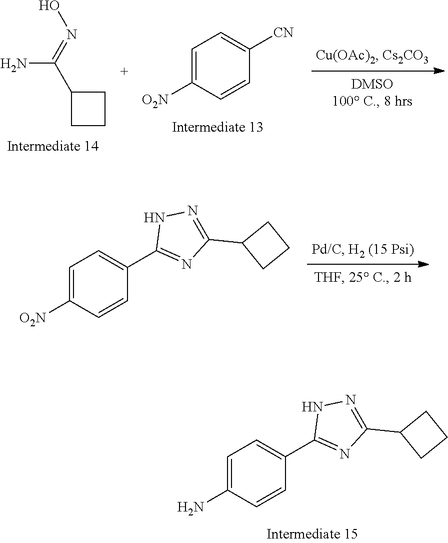 [SMILES: N/C(=N\O)C1CCC1 ; N#Cc1ccc([N+](=O)[O-])cc1 ; O=[N+]([O-])c1ccc(-c2nc(C3CCC3)n[nH]2)cc1 ; Nc1ccc(-c2nc(C3CCC3)n[nH]2)cc1]

A mixture of Intermediate 13 (1.04 g, 7.01 mmol), Intermediate 14 (800 mg, 7.01 mmol), Cs₂CO₃ (6.85 g, 21.0 mmol), Cu(OAc)₂ (381 mg, 2.10 mmol) and Na₂SO₄ (6.97 g, 49.0 mmol, 4.98 mL) in DMSO (30 mL) was degassed and purged with nitrogen 3 times. The mixture was then stirred at 100° C. for 8 h under nitrogen atmosphere before being quenched by addition water 50 mL at 0′C. This was then extracted with EtOAc (2×50 mL) and the combined organic layers were washed with water (2×25 mL), dried over Na₂SO₄, filtered and concentrated under reduced pressure. The residue was purified by Purification Method L to afford 3-cyclobutyl-5-(4-nitrophenyl)-1H-1,2,4-triazole (500 mg, 27%) as a white solid.

¹H NMR (400 MHz, DMSO-d₆) δ 8.07-8.46 (m, 4H), 3.66-3.74 (m, 1H), 2.23-2.42 (m, 4H), 1.86-2.14 (m, 2H).

A mixture of 3-cyclobutyl-5-(4-nitrophenyl)-1H-1,2,4-triazole (400 mg, 1.64 mmol) and 10% Pd/C (100 mg) in THF (20 mL) was degassed and purged with H₂ for 3 times, and then the mixture was stirred at 25° C. for 2 h under H₂ atmosphere. The reaction mixture was filtered and the filtrate was concentrated under reduced pressure. The residue was purified by Purification Method M to afford Intermediate 15 (150 mg, 40% yield) as a white solid.

The data for Intermediate 15 are in Table 3.

### Route 4

### Typical Procedure for the Preparation of Oxazole Anilines, as Exemplified by the Preparation of Intermediate 21, 4-(4-cyclopropyl-5-methyloxazol-2-yl)aniline

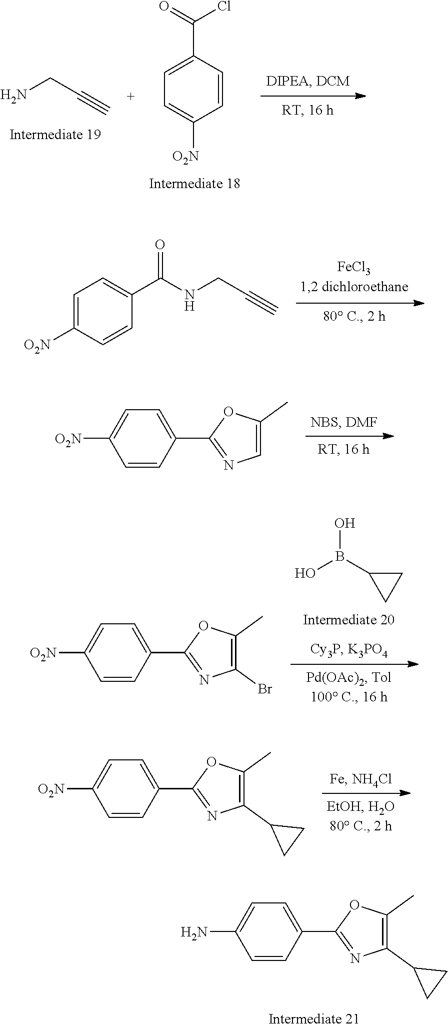

To a solution of Intermediate 18 (1.00 g, 5.39 mmol) in DCM (10 mL) at 0° C. was added DIPEA (1.41 mL, 8.08 mmol) and Intermediate 19 (0.414 mL, 6.47 mmol) and this was stirred at RT for 16 h. The reaction mixture was diluted with water (5 mL) and extracted with DCM (2×10 mL). Combined organic layers were washed with brine (5 mL), dried over Na₂SO₄, and concentrated under reduced pressure to afford 4-nitro-N-(prop-2-yn-1-yl)benzamide (800 mg, 72%) a yellow solid.

LCMS (System 1, Method D): m/z 205 (M+H)⁺ (ESI⁺), Rₜ 1.81 min, 99.6%, UV active.

To a stirred solution of 4-nitro-N-(prop-2-yn-1-yl) benzamine (800 mg, 3.92 mmol) in 1,2-dichloroethane (15 mL) was added iron(III) chloride (318 mg, 1.95 mmol) and this was refluxed at 80° C. for 2 h. The reaction mixture was concentrated under reduced pressure and the residue was purified by flash column chromatography (eluted with Pet-ether:EtOAc (100:0 to 70:30)). The desired fractions were combined and concentrated under reduced pressure to afford 5-methyl-2-(4-nitrophenyl)oxazole (490 mg, 60%) as white solid.

LCMS (System 1, Method C): m/z 205 (M+H)⁺ (ESI⁺), Rₜ 2.15 min, 99.5%, UV active.

To a stirred solution of 5-methyl-2-(4-nitrophenyl) oxazole (440 mg, 2.15 mmol) in DMF (10 mL) at 0° C. was added NBS (767 mg, 4.31 mmol) in portion wise and stirred at RT for 16 h. After the consumption of the starting material the reaction mixture was poured onto crushed ice. The solid obtained was filtered, washed with water, and dried under a high vacuum to afford 4-bromo-5-methyl-2-(4-nitrophenyl)oxazole (360 mg, 41%) as pale yellow solid.

LCMS (System 1, Method C): m/z 282 (M+H)⁺ (ESI⁺), Rₜ 2.58 min, 70.6%, UV active.

To a stirred solution of 4-bromo-5-methyl-2-(4-nitrophenyl)oxazole (335 mg, 1.18 mmol) in toluene (5 mL) were added Intermediate 20 (305 mg, 3.55 mmol), tricyclohexylphosphine (66.4 mg, 0.237 mmol) and tripotassium phosphate (502 mg, 2.36 mmol). The reaction mixture was degassed for 10 min before Pd(OAc)₂ (26.6 mg, 0.118 mmol) was added and the reaction heated at 100° C. for 16 h. After the consumption of the starting material the reaction mixture was filtered through celite. The filtrate was concentrated under reduced pressure and the residue was purified by flash column chromatography (eluted with Pet-ether:EtOAc (100:0 to 0:100)). The desired fractions were combined and concentrated under reduced pressure to afford 4-cyclopropyl-5-methyl-2-(4-nitrophenyl)oxazole (130 mg, 43%) as a yellow solid.

LCMS (System 1, Method C): m/z 245 (M+H)⁺ (ESI⁺), Rₜ 2.73 min, 96.2%, UV active.

To a stirred suspension of 4-cyclopropyl-5-methyl-2-(4-nitrophenyl)oxazole (130 mg, 0.532 mmol) in EtOH (3 mL) and water (1 mL) were added iron (149 mg, 2.66 mmol) and ammonium chloride (142 mg, 2.66 mmol). The reaction mixture was heated at 80° C. for 2 h. After the consumption of the starting material the reaction mixture was filtered through celite and washed with MeOH. The filtrate was evaporated under reduced pressure and dried under high vacuum to afford Intermediate 21 (110 mg, 94%) as a yellow gum.

The data for Intermediate 21 are in Table 3.

### Route 5

### Typical Procedure for the Preparation of 3-Alkyl Isoxazole Anilines, as Exemplified by the Preparation of Intermediate 27, 4-(3-cyclopropylisoxazol-5-yl)aniline

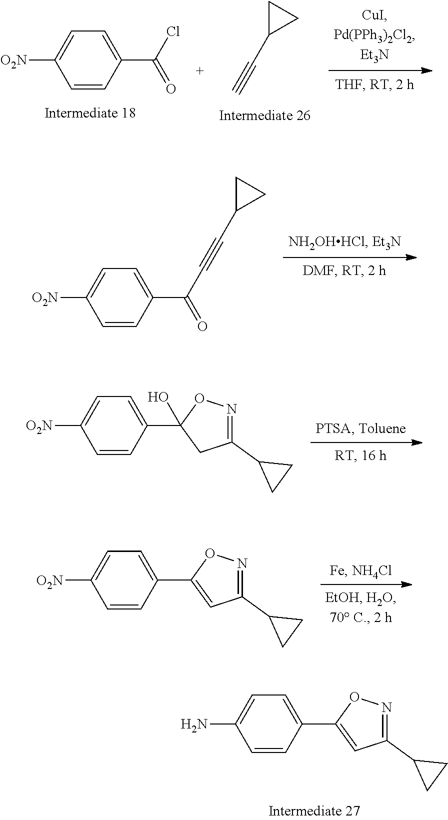

To a degassed solution of Intermediate 18 (2.00 g, 10.78 mmol) in THF (40 ml) were added Intermediate 26 (712 mg, 10.7 mmol), copper(I) iodide (82 mg, 0.431 mmol), bis(triphenylphosphine)palladium(II) chloride (302 mg, 0.431 mmol) and Et₃N (2.25 ml, 16.2 mmol) and this was stirred at RT for 2 h. After the consumption of the starting material the reaction mixture was filtered through celite and washed with ethyl acetate. The filtrate was concentrated under reduced pressure and the residue was purified by flash column chromatography (50 g silica, eluted with Pet-ether:EtOAc (100:0 to 90:10)). The desired fractions were combined and concentrated under reduced pressure to afford 3-cyclopropyl-1-(4-nitrophenyl)prop-2-yn-1-one (1.6 g, 68%) as a brown solid.

¹H NMR (400 MHz, Chloroform-d) δ 8.36-8.33 (m, 2H), 8.29-8.26 (m, 2H), 1.64-1.57 (m, 1H), 1.17-1.08 (m, 4H).

To a stirred solution of 3-cyclopropyl-1-(4-nitrophenyl)prop-2-yn-1-one (500 mg, 2.32 mmol) in DMF (5 mL) were added hydroxylamine hydrochloride (242 mg, 3.49 mmol) and Et₃N (0.65 mL, 4.65 mmol) and this was stirred at RT for 2 h. After the consumption of the starting material the reaction mixture was diluted with EtOAc (10 mL), washed with water (5 mL), dried over Na₂SO₄, and evaporated to afford 3-cyclopropyl-5-(4-nitrophenyl)-4,5-dihydroisoxazol-5-ol (520 mg, 88%) as a yellow solid.

LCMS (System 1, Method C): m/z 249 (M+H)⁺ (ESI⁺), R 1.72 min, 97.7%, UV active.

To a stirred solution of 3-cyclopropyl-5-(4-nitrophenyl)-4,5-dihydroisoxazol-5-ol (620 mg, 2.49 mmol) in toluene (5 mL) was added p-toluenesulfonic acid monohydrate (47.5 mg, 0.250 mmol) and this was stirred at RT for 16 h. After the consumption of the starting material the reaction mixture was evaporated to dryness under reduced pressure. The residue was purified by flash column chromatography (25 g silica, eluted with Pet-ether:EtOAc (100:0 to 70:30)). The desired fractions were combined and concentrated under reduced pressure to afford 3-cyclopropyl-5-(4-nitrophenyl)-isoxazole (520 mg, 88%) as a pale yellow solid.

LCMS (System 1, Method C): m/z 231 (M+H)⁺ (ESI⁺), Rₜ 2.41 min, 99.4%, UV active.

To a stirred solution of 3-cyclopropyl-5-(4-nitrophenyl)-isoxazole (150 mg, 0.652 mmol) in EtOH (3 mL) and water (1 mL) were added iron (182 mg, 3.26 mmol) and ammonium chloride (174 mg, 3.26 mmol) and this was heated at 70° C. for 2 h. After the consumption of the starting material the reaction mixture was diluted with water (20 mL) and extracted with EtOAc (3×10 mL). The combined organic extracts were washed with brine (10 mL), dried over Na₂SO₄, and concentrated under reduced pressure to afford Intermediate 27 (120 mg, 89%) as a brown solid.

The data for Intermediate 27 are in Table 3.

### Route 6

### Typical Procedure for the Preparation of 5-Alkyl Isoxazole Anilines, as Exemplified by the Preparation of Intermediate 29, 4-(5-cyclopropylisoxazol-3-yl)aniline

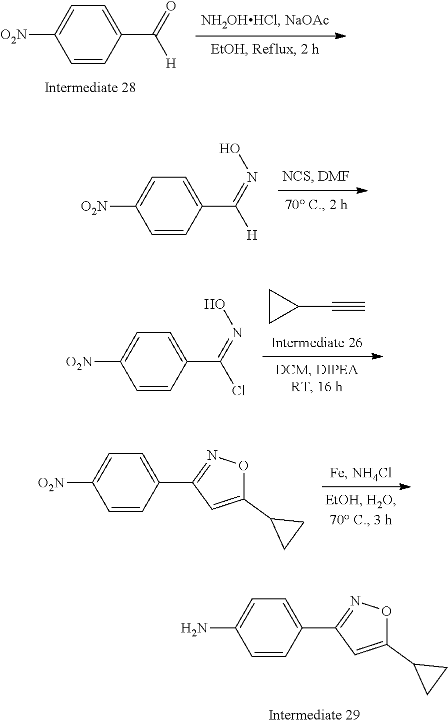

To a stirred solution of Intermediate 28 (1.00 g, 6.62 mmol) in EtOH (9 mL) and water (3 mL) were added sodium acetate (1.35 g, 16.5 mmol) and hydroxylamine hydrochloride (0.68 g, 9.79 mmol) at RT then heated at 70° C. for 2 h. After the consumption of the starting material the reaction mixture was evaporated under reduced pressure, diluted with EtOAc (30 mL), washed with water (10 mL) and dried over Na₂SO₄ to afford 4-nitrobenzaldehyde oxime (850 mg, 77%) as a yellow solid.

¹H NMR (400 MHz, DMSO-d₆) δ 11.86 (br s, 1H), 8.31-8.25 (m, 3H), 7.88-7.85 (m, 2H).

To a stirred solution of 4-nitrobenzaldehyde oxime (500 mg, 3.01 mmol) in DMF (5 mL) was added NCS (442 mg, 3.31 mmol) and this was heated at 70° C. for 2 h. After the consumption of the starting material the reaction mixture was diluted with EtOAc (20 mL), washed with water (5 mL), dried over Na₂SO₄ and evaporated to afford N-hydroxy-4-nitrobenzimidoyl chloride (500 mg, 83%) as brown gum.

To a stirred solution of N-hydroxy-4-nitrobenzimidoyl chloride (500 mg, 2.49 mmol) in DCM (5 mL) at 0° C. were added Intermediate 26 (0.21 mL, 2.49 mmol) and DIPEA (0.57 mL, 3.24 mmol). The reaction mixture was stirred at RT for 16 h. After the consumption of the starting material the reaction mixture was diluted with water (3 mL) and extracted with DCM (3×5 mL). The combined organic extracts were washed with brine (10 mL), dried over anhydrous Na₂SO₄, filtered and concentrated under reduced pressure. The residue was purified by flash column chromatography (25 g silica, eluted with Pet-ether:EtOAc (100:0 to 80:20)). The desired fractions were combined and concentrated under reduced pressure to afford 5-cyclopropyl-3-(4-nitrophenyl)isoxazole (180 mg, 20%) as a pale yellow solid.

LCMS (System 1, Method C): m/z 231 (M+H)⁺ (ESI⁺), Rₜ 2.43 min, 64.4%, UV active.

To a stirred solution of 5-cyclopropyl-3-(4-nitrophenyl)isoxazole (180 mg, 0.504 mmol) in EtOH (3 mL) and water (1 ml) were added iron (141 mg, 2.52 mmol) and ammonium chloride (135 mg, 2.52 mmol). The reaction mixture was heated at 75° C. for 4 h. After the consumption of the starting material the reaction mixture was evaporated to dryness. The residue was filtered through celite using EtOAc (20 mL). The filtrate was washed with water (5 mL), dried over Na₂SO₄ and concentrated under reduced pressure to afford Intermediate 29 (120 mg, 93%) as a brown gum.

The data for Intermediate 29 are in Table 3.

### Route 7

### Typical Procedure for the Preparation of 1,3,4-Oxadiazole Anilines, as Exemplified by the Preparation of Intermediate 32, 4-(5-cyclopropyl-1,3,4-oxadiazol-2-yl)aniline

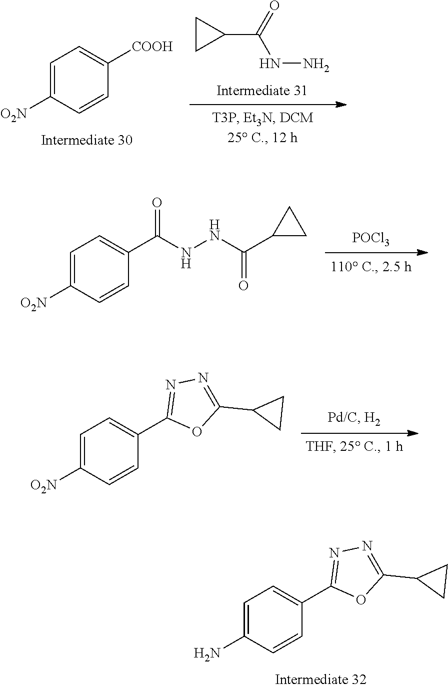

To a solution of Intermediate 30 (1.00 g, 5.98 mmol) in DCM (20 mL) was added T3P (7.12 mL, 11.9 mmol, 50% purity) and Intermediate 31 (599 mg, 5.98 mmol). Et₃N (2.50 mL, 17.9 mmol) was then added at 0° C. and the mixture was stirred at 25° C. for 12 h. After the consumption of the starting material the reaction mixture was quenched by addition of H₂O (50 mL), then extracted with ethyl acetate (3×50 mL). The combined organic layers were dried over Na₂SO₄, filtered and concentrated under reduced pressure to give a residue. The residue was recrystallized with water to obtain N-(cyclopropanecarbonyl)-4-nitrobenzohydrazide (800 mg, 53%) as a white solid.

¹H NMR (400 MHz, Methanol-d₄) δ 8.34 (br d, J=8.34 Hz, 2H), 8.08 (br d, J=8.34 Hz, 2H), 1.73 (br s, 1H), 0.80-1.07 (m, 4H),

A solution of N-(cyclopropanecarbonyl)-4-nitrobenzohydrazide (500 mg, 2.01 mmol) in POCl₃ (6.25 mL, 66.2 mmol) was stirred at 110° C. for 2.5 h. The reaction mixture was quenched by addition of water (10 mL) at 0° C. and then extracted with EtOAc (3×30 mL). The combined organic layers were dried over Na₂SO₄, filtered and concentrated under reduced pressure to give a residue. The residue was recrystallized in water to obtain 2-cyclopropyl-5-(4-nitrophenyl)-1,3,4-oxadiazole (300 mg, 61%) as a yellow solid.

LCMS (System 2, Method E): m/z 231 (M+H)⁺ (ESI⁺), Rₜ 1.053 min, 95%, UV active.

To a solution of 2-cyclopropyl-5-(4-nitrophenyl)-1,3,4-oxadiazole (200 mg, 0.865 mmol) in THF (10 mL) was added Pd/C (921 mg, 0.865 mmol) at 25° C. under H₂ balloon (15 psi). The reaction was stirred at 25° C. for 1 h. After the consumption of the starting material the reaction mixture was filtered and the filtrate was concentrated under reduce pressure to obtain Intermediate 32 (137 mg, 79%) as a white solid.

The data for Intermediate 32 are in Table 3.

### Route 8

### Typical Procedure for the Preparation of 1,2,4-Oxadiazole Anilines, as Exemplified by the Preparation of Intermediate 34, 4-(3-cyclopropyl-1,2,4-oxadiazol-5-yl)aniline

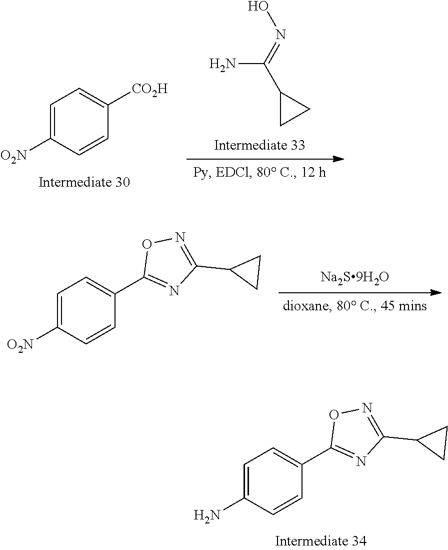 [SMILES: O=C(O)c1ccc([N+](=O)[O-])cc1 ; N/C(=N\O)C1CC1 ; O=[N+]([O-])c1ccc(-c2nc(C3CC3)no2)cc1 ; Nc1ccc(-c2nc(C3CC3)no2)cc1]

To a solution of Intermediate 30 (1.50 g, 8.98 mmol) in pyridine (20 mL) was added EDCl (3.44 g, 17.9 mmol) and Intermediate 33 (898 mg, 8.98 mmol). The reaction mixture was stirred at 80° C. for 12 h before being concentrated under reduced pressure. The residue was purified by recrystallization from water (100 mL) at 25° C. to give 3-cyclopropyl-5-(4-nitrophenyl)-1,2,4-oxadiazole (539 mg, 24%) as a yellow solid.

¹H NMR (400 MHz, DMSO-d₆) δ 8.39-8.44 (m, 2H), 8.28-8.32 (m, 2H), 2.18-2.27 (m, 1H), 1.11-1.19 (m, 2H), 0.98-1.05 (m, 2H).

To a solution of 3-cyclopropyl-5-(4-nitrophenyl)-1,2,4-oxadiazole (390 mg, 1.69 mmol in dioxane (8 mL) at 80° C. was added a hot solution (80° C.) of Na₂S·9H₂O (1.22 g, 5.06 mmol) in water (8 mL). The resulting mixture was stirred at 80° C. for 45 mins before being concentrated under reduced pressure. The residue was diluted with H₂O (20 mL) and extracted with EtOAc (3×20 mL). The combined organic layers were washed with water (3×20 mL, dried over Na₂SO₄, filtered and concentrated under reduced pressure. The residue was purified by Prep-TLC (Pet-ether:EtOAc=1:2) to afford Intermediate 34 (280 mg, 65%) as a yellow solid.

The data for Intermediate 34 are in Table 3.

### Route 9

### Typical Procedure for the Preparation of 1,2,4-Oxadiazole Anilines, as Exemplified by the Preparation of Intermediate 36, 4-(5-cyclopropyl-1,2,4-oxadiazol-3-yl)aniline

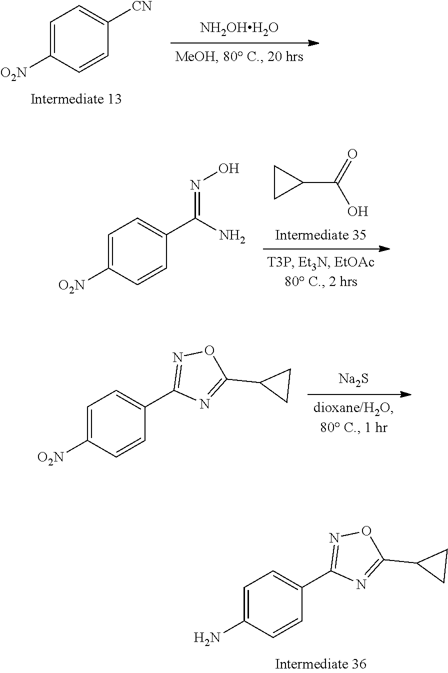

To a solution of Intermediate 13 (5.00 g, 33.7 mmol) in MeOH (100 mL) was added NH₂OH (4.46 g, 67.51 mmol, 50%6 purity) in H₂O (8 mL). The mixture was stirred at 80° C. for 20 h before being concentrated under reduced pressure to give N-hydroxy-4-nitrobenzimidamide (4.50 g, 66%6) as a light yellow solid.

¹H NMR (400 MHz, DMSO-d₆) δ 10.15 (s, 1H), 8.24 (d, J=9.0 Hz, 2H), 7.79-8.06 (m, 2H), 6.08 (br s, 2H1).

A mixture of N-hydroxy-4-nitrobenzimidamide (1.00 g, 5.52 mmol), Intermediate 35 (654 μL, 8.28 mmol), T3P (8.21 mL, 13.8 mmol, 50% purity) and Et₃N (3.84 mL, 27.6 mmol) in EtOAc (5 mL) was stirred at 80° C. for 2 h. The reaction mixture was poured into water (50 mL) and extracted with EtOAc (2×50 mL). The combined organic phases was concentrated under reduced pressure and the residue was purified by flash column chromatography (eluted with Pet-ether:EtOAc (50:50)). The desired fractions were combined and concentrated under reduced pressure to afford 5-cyclopropyl-3-(4-nitrophenyl)-1,2,4-oxadiazole (500 mg, 35) as a yellow solid.

LCMS (System 2, Method E): m/z 231 (M+H)⁺ (ESI⁺), Rₜ 1.259 min, 90%, UV active.

A mixture of 5-cyclopropyl-3-(4-nitrophenyl)-1,2,4-oxadiazole (400 mg, 1.56 mmol) in dioxane (10 mL) was heated to 80° C. and stirred for 30 minutes. A mixture of sodium sulfide nonahydrate (1.12 g, 4.67 mmol) in water (10 ml) was heated to 80° C. and stirred for 30 minutes. The two mixtures were then mixed together and stirred at 80° C. for 1 h before the reaction mixture was concentrated under reduced pressure. The residue was purified by flash column chromatography (eluted with Pet-ether:EtOAc (50:50)). The desired fractions were combined and concentrated under reduced pressure to afford Intermediate 36 (250 mg, 75%) as a white solid.

The data for Intermediate 36 are in Table 3.

### Route 10

### Typical Procedure for the Preparation of 4-(1H-pyrazol-3/5-yl)anilines, as Exemplified by the Preparation of Intermediate 50, 4-(3-cyclopropyl-1H-pyrazol-5-yl)aniline

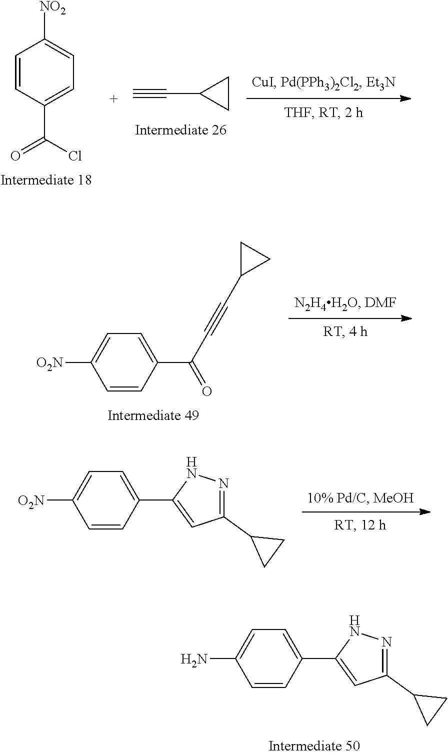

To a degassed solution of Intermediate 18 (2.00 g, 10.7 mmol) in THF (40 ml) were added Intermediate 26 (712 mg, 10.7 mmol), copper(I) iodide (82 mg, 0.431 mmol), bis(triphenylphosphine)palladium(II) chloride (302 mg, 0.431 mmol) and Et₃N (2.25 ml, 16.2 mmol) and this was stirred at RT for 2 h. After the consumption of the starting material the reaction mixture was filtered through celite and washed with EtOAc. The filtrate was evaporated under reduced pressure and the residue was purified by flash column chromatography (50 g silica, eluted with Pet-ether:EtOAc (100:0 to 90:10)). The desired fractions were combined and concentrated under reduced pressure to afford Intermediate 49 (1.6 g, 68%) as a brown solid.

¹H NMR (400 MHz, Chloroform-d) δ 8.36-8.33 (m, 2H), 8.29-8.26 (m, 2H), 1.64-1.57 (m, 1H), 1.17-1.08 (m, 4H).

To a stirred solution of Intermediate 49 (250 mg, 1.16 mmol) in DMF (2.5 ml) was added hydrazine hydrate (70 mg, 1.39 mmol). The reaction mixture was stirred at RT for 4 h. After the consumption of the starting material the reaction mixture was poured into crushed ice. The solid obtained was filtered and dried under vacuum to afford 3-cyclopropyl-5-(4-nitrophenyl)-1H-pyrazole (205 mg, 77%) as a pale yellow solid.

LCMS (System 1, Method F): m/z 230 (M+H)⁺ (ESI⁺), Rₜ 1.98 min, 99%, UV active.

To a stirred solution of 3-cyclopropyl-5-(4-nitrophenyl)-1H-pyrazole (200 mg, 0.872 mmol) in MeOH (10 ml) was added 10% Pd/C (50 mg, 0.470 mmol). The reaction mixture was stirred at 1 kg of H₂ pressure at RT for 4 h. After the consumption of the starting material the reaction mixture was filtered through celite and washed with MeOH. The filtrate was evaporated under reduced pressure and the resulting residue dried under high vacuum to afford Intermediate 50 (169 mg, 92%) as brown gum.

The data for Intermediate 50 are in Table 3.

### Route 11

### Typical Procedure for the Preparation of 4-(3-benzyl-1H-pyrazol-5-yl)anilines, as Exemplified by the Preparation of Intermediate 52, 4-(3-benzyl-1H-pyrazol-6-yl)aniline

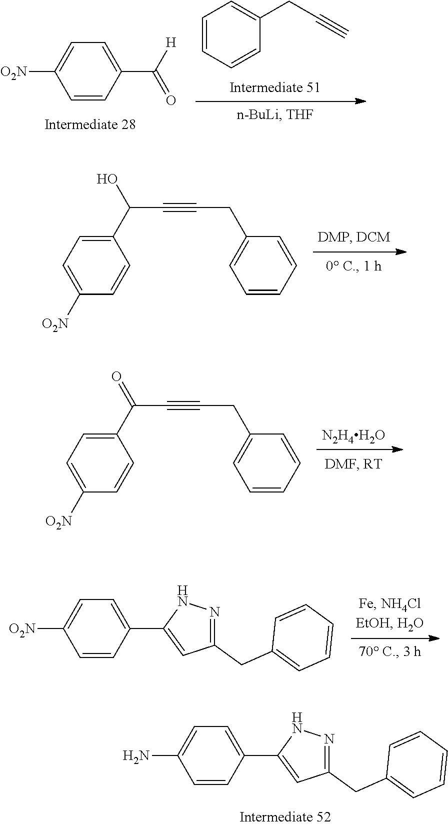

To a stirred solution of Intermediate 51 (846 mg, 7.28 mmol) in THF (5 mL) at −78° C. was added 1.6 M n-butyllithium (4.96 mL, 7.94 mmol) and stirred for 1 h. Intermediate 28 (1.00 g, 6.62 mmol) in THF (5 mL) was added to the reaction mixture at −78° C. dropwise and stirred for 1 h. After the consumption of the starting material the reaction mixture was quenched with aqueous NH₄Cl (10 mL) and extracted with EtOAc (2×15 mL). The combined organic extracts were washed with brine (10 mL), dried over anhydrous Na₂SO₄, filtered, and concentrated under reduced pressure. The residue was purified by flash column chromatography (25 g silica, eluted with Pet-ether:EtOAc (100:0 to 80:20)). The desired fractions were combined and concentrated under reduced pressure to afford 1-(4-nitrophenyl)-4-phenylbut-2-yn-1-ol (1.1 g, 61%) as a yellow oil.

¹H NMR (300 MHz, DMSO-d₆) δ 8.25 (d, J=8.4 Hz, 2H), 7.75 (d, J=8.4 Hz, 2H), 7.27-7.30 (m, 5H), 6.33 (d, J=5.70 Hz, 1H), 5.61-5.59 (m, 1H), 3.72 (d, J=1.8 Hz, 2H)

To a stirred solution of 1-(4-nitrophenyl)-4-phenylbut-2-yn-1-ol (0.50 g, 1.87 mmol) in DCM (10 mL) at 0° C. was added DMP (1.19 g, 2.81 mmol) and reaction mixture was stirred at 0° C. for 1 h. After the consumption of the starting material the reaction mixture was filtered through celite. The filtrate was washed with aqueous 10% NaHCO₃ solution, dried over Na₂SO₄ and evaporated under reduced pressure. The residue was purified by flash column chromatography (25 g silica, eluted with Pet-ether:EtOAc (100:0 to 70:30)). The desired fractions were combined and concentrated under reduced pressure to afford 1-(4-nitrophenyl)-4-phenylbut-2-yn-1-one (280 mg, 56%) as a yellow solid.

¹H NMR (400 MHz, Chloroform-d) δ 8.36-8.31 (m, 4H), 7.43-7.29 (m, 5H), 3.98 (s, 2H).

To a stirred solution of 1-(4-nitrophenyl)-4-phenylbut-2-yn-1-one (280 mg, 1.05 mmol) in DMF (3 mL) was added hydrazine hydrate (63 mg, 1.26 mmol). The reaction mixture was stirred at RT for 16 h. After the consumption of the starting material the reaction mixture was poured into crushed ice. The solid obtained was filtered and dried under vacuum to afford 3-benzyl-5-(4-nitrophenyl)-1H-pyrazole (250 mg, 85%) as a pale yellow solid.

¹H NMR (300 MHz, DMSO-d₆) δ 13.13 (br s, 1H), 8.25-8.22 (m, 2H), 8.22-7.96 (m, 2H), 7.31-7.23 (m, 5H), 6.68 (s, 1H), 4.03 (s, 2H).

To a stirred solution of 3-benzyl-5-(4-nitrophenyl)-1H-pyrazole (250 mg, 0.89 mmol) in EtOH (3 mL) and water (1 mL) were added iron (150 mg, 2.69 mmol) and NH₄Cl (144 mg, 2.69 mmol). The reaction mixture was heated at 70° C. for 3 h. After the consumption of the starting material the reaction mixture was evaporated to dryness under reduced pressure. The residue was filtered through celite using EtOAc (20 mL) and then the filtrate was extracted with aqueous 1.5 N HCl (2×5 mL). The combined aqueous layers were washed with EtOAc (5 mL) then basified with 10% NaHCO₃ solution. The aqueous solution was extracted with EtOAc (20 mL) then the organic layer was dried over Na₂SO₄ and concentrated to under reduced pressure to afford Intermediate 52 (120 mg, 47%) as a yellow solid.

The data for Intermediate 52 are in Table 3.

### Route 12

### Typical Procedure for the Preparation of Substituted 4-(5-cyclopropyl-1H-pyrazol-3-yl)anilines, as Exemplified by the Preparation of Intermediate 53, 4-(5-cyclopropyl-4-methyl-1-((2-(trimethylsilyl)ethoxy)methyl)-1H-pyrazol-3-yl)aniline

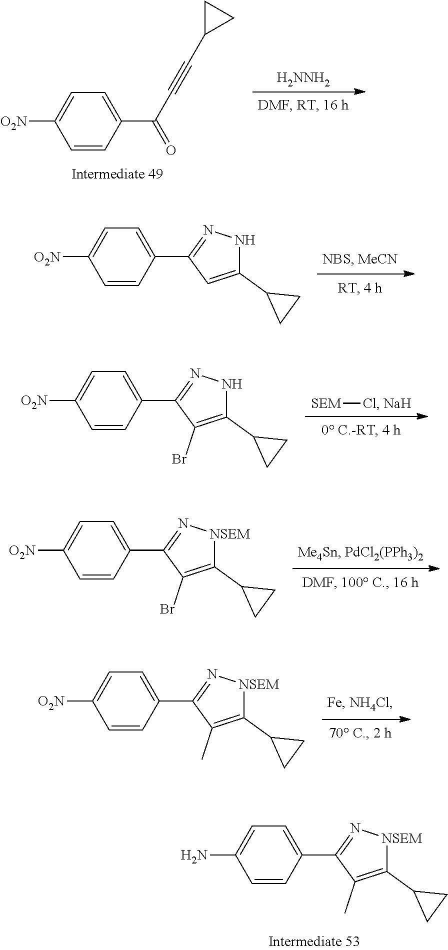

To a stirred solution of Intermediate 49 (1.2 g, 3.79 mmol) in DMF (3 mL) was added hydrazine hydrate (0.228 mL, 4.55 mmol) then the reaction mixture was stirred at RT for 16 h. After the consumption of the starting material the reaction mixture was diluted with water (15 mL), extracted with EtOAc (2×20 mL) and concentrated under reduced pressure to afford crude. The residue was purified by flash column chromatography (eluted with Pet-ether:EtOAc (100:0 to 40:60)). The desired fractions were combined and concentrated under reduced pressure to afford 3-cyclopropyl-5-(4-nitrophenyl)-1H-pyrazole (685 mg, 73%) as a brown solid.

LCMS (System 1, Method H): m/z 228 (M−H)⁻ (ESI⁻), Rₜ 2.10 min, 92%, UV active.

To a stirred solution of 3-cyclopropyl-5-(4-nitrophenyl)-1H-pyrazole (685 mg, 2.99 mmol) in MeCN (5 mL) was added NBS (745 mg, 4.18 mmol) at RT. The reaction mixture was stirred at RT for 4 h. After the consumption of the starting material the reaction mixture was diluted in EtOAc (3×20 mL) and washed with water (20 mL). The organic layer was concentrated under reduced pressure and the residue was purified by flash column chromatography (25 g silica, eluted with Pet-ether:EtOAc (100:0 to 50:50)). The desired fractions were combined and concentrated under reduced pressure to afford 4-bromo-3-cyclopropyl-5-(4-nitrophenyl)-1H-pyrazole (720 mg, 76%) as a yellow solid.

LCMS (System 1, Method D): m/z 309 (M+H)⁺ (ESI⁺), Rₜ 2.99 min, 97%, UV active.

To a stirred solution of 4-bromo-3-cyclopropyl-5-(4-nitrophenyl)-1H-pyrazole (355 mg, 1.15 mmol) in THF (3 mL) maintained at 0° C. were added NaH (69.1 mg, 1.72 mmol) and SEM-Cl (0.245 mL, 1.38 mmol). The reaction mixture was stirred at RT for 4 h. After the consumption of the starting material the reaction mixture was quenched with ice-cold water (10 mL), extracted with EtOAc (3×15 mL). The organic layer was concentrated under reduced pressure and the residue was purified by flash column chromatography (25 g silica, eluted with Pet-ether:EtOAc (100:0 to 50:50)). The desired fractions were combined and concentrated under reduced pressure to afford 4-bromo-3-cyclopropyl-5-(4-nitrophenyl)-1-((2-(trimethylsilyl)ethoxy)methyl)-1H-pyrazole (mixture of SEM regioisomers) (460 mg, 91%) as a yellow gum.

LCMS (System 1, Method D): m/z 440 (M+H)⁺ (ESI⁺), Rₜ 3.87 min, 38% and Rₜ 3.93 min, 61%, UV active.

A solution of 4-bromo-5-cyclopropyl-3-(4-nitrophenyl)-1-((2-(trimethylsilyl)ethoxy)methyl)-1H pyrazole (mixture of SEM regioisomers) (450 mg, 1.02 mmol) in DMF (5 mL) in a sealed tube at RT was bubbled with argon for 5 min. Tetramethyltin (0.213 mL, 1.54 mmol) and bis(triphenylphosphine)palladium(II) chloride (72.0 mg, 0.103 mmol) were added to the reaction mixture and heated at 100° C. for 16 h. After the consumption of the starting material the reaction mixture was evaporated to dryness under reduced pressure. The residue was purified by flash column chromatography (25 g silica, eluted with Pet-ether:EtOAc (100:0 to 70:30)). The desired fractions were combined and concentrated under reduced pressure to afford 5-cyclopropyl-4-methyl-3-(4-nitrophenyl)-1-((2-(trimethylsilyl)ethoxy)methyl)-1H-pyrazole (300 mg, 66%) as a brown gum.

LCMS (System 1, Method D): m/z 374 (M+H)⁺ (ESI⁺), Rₜ 3.22 min, 85%, UV active.

To a stirred solution of 5-cyclopropyl-4-methyl-3-(4-nitrophenyl)-1-((2-(trimethylsilyl)ethoxy)methyl)-1H-pyrazole (220 mg, 0.589 mmol) in EtOH (5 mL) and water (2 mL) were added iron (164 mg, 2.94 mmol) and ammonium chloride (158 mg, 2.94 mmol) were added at RT. The reaction mixture was stirred at 90° C. for 2 h. After the consumption of the starting material the reaction mixture was concentrated under reduced pressure and then filtered through celite using EtOAc. The filtrate was diluted with water (10 mL) and extracted with EtOAc. The combined organic extracts was washed with brine (20 mL), dried over anhydrous Na₂SO₄, filtered and concentrated under reduced pressure to afford desired product Intermediate 53 (160 mg, 58%) as a brown gum.

The data for Intermediate 53 are in Table 3.

### Route 13

### Typical Procedure for the Preparation of Substituted 4-(5-cyclopropyl-1H-pyrazol-3-yl)anilines, as Exemplified by the Preparation of Intermediate 54, 4-(5-cyclopropyl-1-methyl-1H-pyrazol-3-yl)aniline

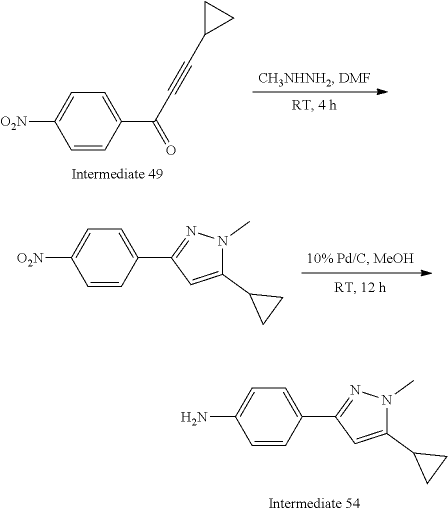

To a stirred solution of Intermediate 49 (250 mg, 1.16 mmol) in DMF (2.5 ml) was added methylhydrazine (64 mg, 1.39 mmol). The reaction mixture was stirred at RT for 4 h. After the consumption of the starting material the reaction mixture was poured into crushed ice. The solid obtained was filtered and dried under vacuum to afford 3-cyclopropyl-1-methyl-5-(4-nitrophenyl)-1H-pyrazole (234 mg, 80%) as a yellow solid.

LCMS (System 1, Method F): m/z 244 (M+H)⁺ (ESI⁺), Rₜ 2.25 min, 96%, UV active.

To a stirred solution of 3-cyclopropyl-1-methyl-5-(4-nitrophenyl)-1H-pyrazole (200 mg, 0.82 mmol) in MeOH (10 mL) was added 10% Pd/C (50 mg, 0.470 mmol). The reaction mixture was stirred at 1 kg of H₂ pressure at RT for 4 h. After the consumption of the starting material the reaction mixture was filtered through celite and washed with MeOH. The filtrate was evaporated under reduced pressure and the resulting residue was dried under high vacuum to afford Intermediate 54 (170 mg, 92%) as yellow gum.

The data for Intermediate 54 are in Table 3.

### Route 14

### Typical Procedure for the Preparation of Substituted 4-(5-cyclopropyl-1-methyl-1 i pyrazol-3-yl)anilines, as Exemplified by the Preparation of Intermediate 55, 4-(5-cyclopropyl-1,4-dimethyl-1H-pyrazol-3-yl)aniline

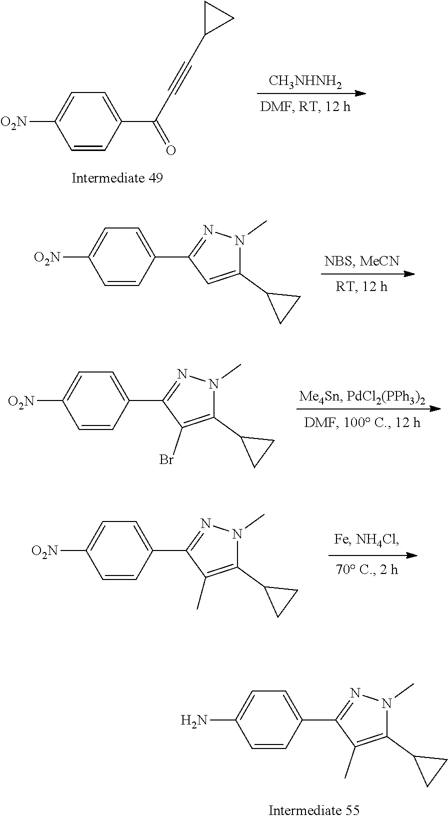

To a stirred solution of Intermediate 49 (1.00 g, 4.65 mmol) in DMF (10 mL) was added methylhydrazine (321 mg, 6.97 mmol). The reaction mixture was stirred at RT for 4 h. After the consumption of the starting material the reaction mixture was poured into crushed ice. The solid obtained was filtered and dried under vacuum to afford 3-cyclopropyl-1-methyl-5-(4-nitrophenyl)-1H-pyrazole (650 mg, 56%) as a yellow solid.

LCMS (System 1, Method C): m/z 244 (M+H)⁺ (ESI⁺), Rₜ 2.39 min, 98%, UV active.

To a stirred solution of 3-cyclopropyl-1-methyl-5-(4-nitrophenyl)-1H-pyrazole (600 mg, 2.46 mmol) in MeCN (10 mL) at RT was added NBS (600 mg, 3.37 mmol) and the reaction mixture was stirred at RT for 16 h. After the consumption of the starting material the reaction mixture was diluted in EtOAc (50 mL), washed with water (10 mL), dried over Na₂SO₄ and concentrated under reduced pressure. The residue was purified by flash column chromatography (eluted with Pet-ether:EtOAc (100:0 to 80-20)). The desired fractions were combined and concentrated under reduced pressure to afford 4-bromo-3-cyclopropyl-1-methyl-5-(4-nitrophenyl)-1H-pyrazole (300 mg, 37%) as a yellowish solid.

LCMS (System 1, Method C): m/z 322 (M+H)⁺ (ESI⁺), Rₜ 2.73 min, 98%, UV active.

To a stirred solution of 4-bromo-3-cyclopropyl-1-methyl-5-(4-nitrophenyl)-1H-pyrazole (200 mg, 0.621 mmol) in DMF (5 mL) was added tetramethyltin (0.129 mL, 0.931 mmol) and the reaction mixture was purged with nitrogen for 5 min. Bis(triphenylphosphine)palladium(II) chloride (43.6 mg, 0.062 mmol) was added and the reaction mixture was heated at 100° C. for 16 h. After the consumption of the starting material the reaction mixture was filtered through celite and washed with EtOAc (10 mL). The filtrate was evaporated under reduced pressure and the residue was purified by flash column chromatography (25 g silica, eluted with Pet-ether:EtOAc (100:0 to 50:50)). The desired fractions were combined and concentrated under reduced pressure to afford 3-cyclopropyl-1,4-dimethyl-5-(4-nitrophenyl)-1H-pyrazole (120 mg, 64%) as an off white solid.

LCMS (System 1, Method C): m/z 258 (M+H)⁺ (ESI⁺), Rₜ 2.50 min, 86% , UV active.

To a stirred solution of 3-cyclopropyl-1,4-dimethyl-5-(4-nitrophenyl)-1H-pyrazole (200 mg, 0.777 mmol) in EtOH (3 mL) and water (1 mL) were added iron powder (217 mg, 3.89 mmol) and ammonium chloride (208 mg, 3.89 mmol). The reaction mixture was heated at 70° C. for 2 h. After the consumption of the starting material the reaction mixture was filtered through celite and washed with EtOAc (10 mL). The filtrate was concentrated under reduced pressure and the residue dried to afford Intermediate 55 (120 mg, 67%) as a yellow gum.

The data for Intermediate 55 are in Table 3.

### Route 15

### Typical Procedure for the Preparation of Substituted 6-(5-methyloxazol-2-yl)pyridin-3-amines, as Exemplified by the Preparation of Intermediate 58, 6-(4-bromo-5-methyloxazol-2-yl)pyridin-3-amine di-hydrochloride

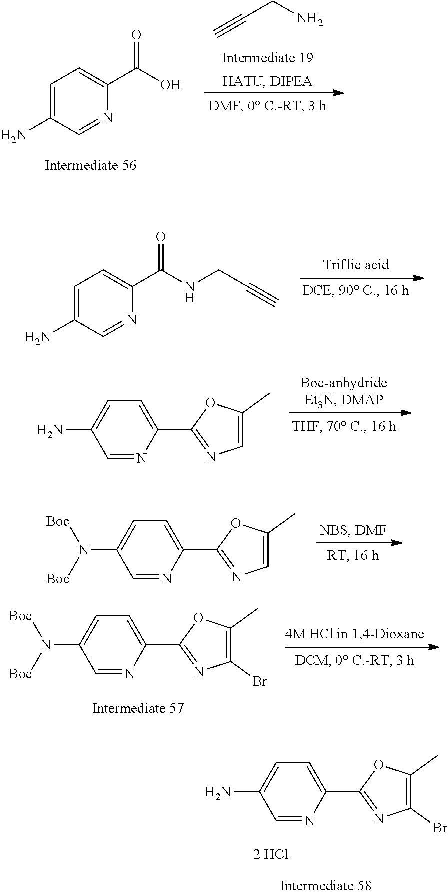

To a stirred solution of Intermediate 56 (2.00 g, 14.48 mmol) in DMF (20 mL) were added DIPEA (7.59 mL, 43.4 mmol), HATU (6.61 g, 17.3 mmol) and Intermediate 19 (1.39 mL, 21.7 mmol) at 0° C. The reaction mixture was stirred at RT for 3 h. After the consumption of the starting material the reaction mixture was quenched with water (10 mL) and extracted with DCM (3×20 mL). The combined organic extracts were washed with brine (10 mL), dried over Na₂SO₄, concentrated under reduced pressure and dried under high vacuum to afford 5-amino-N-(prop-2-yn-1-yl)picolinamide (3.5 g, 72%) as a yellow gum.

LCMS (System 1, Method D): m/z 176 (M+H)⁺ (ESI⁺), Rₜ 1.27 min, 52%, UV active.

To a stirred solution of 5-amino-N-(prop-2-yn-1-yl)picolinamide (1.80 g, 10.2 mmol) in DCE (20 mL) at RT was added triflic acid (9.1 mL, 103 mmol) and the reaction mixture was heated at 90° C. for 16 h. After the consumption of the starting material the reaction mixture was basified with 10% NaHCO₃ solution to pH=8 and extracted with DCM (2×50 mL). The combined organic extracts were washed with brine (20 mL), dried over anhydrous Na₂SO₄, concentrated under reduced pressure and dried under a high vacuum to afford 6-(5-methyloxazol-2-yl)pyridin-3-amine (1.8 g, 91%) as a yellow solid.

LCMS (System 1, Method D): m/z 176 (M+H)⁺ (ESI⁺), Rₜ 1.34 min, 90%, UV active.

To a stirred solution of 6-(5-methyloxazol-2-yl)pyridin-3-amine (1.8 g, 10.2 mmol) in THF (18 mL) at RT were added Et₃N (4.3 mL, 30.8 mmol), DMAP (1.89 g, 15.4 mmol) and Boc-anhydride (2.36 mL, 10.2 mmol). The reaction mixture was stirred at 70° C. for 16 h. After the consumption of the starting material the reaction mixture was quenched with water (20 mL) and extracted with EtOAc (2×30 mL). The combined organic layers were washed with brine (20 mL), dried over anhydrous Na₂SO₄ and concentrated under reduced pressure. The residue was purified by flash column chromatography (25 g silica, eluted with Pet-ether:EtOAc (100:0 to 40:60)). The desired fractions were combined and concentrated under reduced pressure to afford tert-butyl (tert-butoxycarbonyl)(6-(5-methyloxazol-2-yl)pyridin-3-yl)carbamate (1.64 g, 42%) as a yellow solid.

LCMS (System 1, Method D): m/z 376 (M+H)⁺ (ESI⁺), Rₜ 2.62 min, 99%, UV active.

To a stirred solution of tert-butyl (tert-butoxycarbonyl)(6-(5-methyloxazol-2-yl)pyridin-3-yl)carbamate (1.6 g, 4.26 mmol) in DMF (15 mL) at RT was added NBS (1.14 g, 6.39 mmol) and the reaction mixture was stirred at RT for 16 h. After the consumption of the starting material the reaction mixture was quenched with water (20 mL) and extracted with EtOAc (2×30 mL). The combined organic layers were washed with brine (20 mL), dried over anhydrous Na₂SO₄ and concentrated under reduced pressure. The residue was purified by flash column chromatography (eluted with Pet-ether:EtOAc (100:0 to 40:60)). The desired fractions were combined and concentrated under reduced pressure to afford Intermediate 57 (800 mg, 40%) as a white solid.

The data for Intermediate 57 are in Table 3.

To a stirred solution of Intermediate 57 (200 mg, 0.44 mmol) in DCM (2.5 mL) was added 4M HCl in 1,4-dioxane (5 mL, 20 mmol) at 0° C. The resultant reaction mixture was stirred at RT for 3 h. After the consumption of the starting material the reaction mixture was concentrated under reduced pressure to afford Intermediate 58 (145 mg, 99%) as a pale brown solid.

The data for Intermediate 58 are in Table 3.

### Route 16

### Typical Procedure for the Preparation of Substituted 6-(5-methyloxazol-2-yl)pyridin-3-amines, as Exemplified by the Preparation of Intermediate 59, 6-(4-cyclopropyl-5-methyloxazol-2-yl)pyridin-3-amine di-hydrochloride

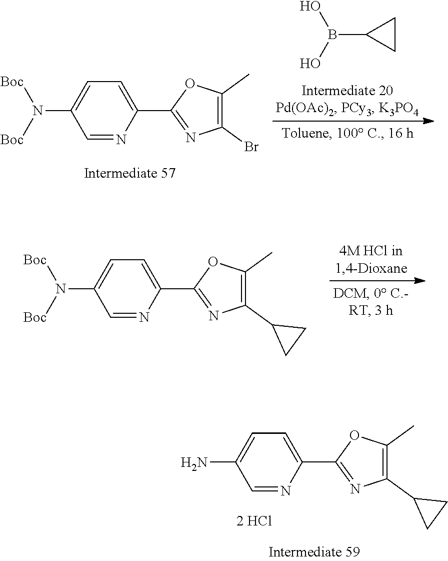 [SMILES: OB(O)C1CC1 ; Cc1oc(-c2ccc(N(C(=O)OC(C)(C)C)C(=O)OC(C)(C)C)cn2)nc1Br ; Cc1oc(-c2ccc(N(C(=O)OC(C)(C)C)C(=O)OC(C)(C)C)cn2)nc1C1CC1 ; Cc1oc(-c2ccc(N)cn2)nc1C1CC1]

To a stirred solution of Intermediate 57 (400 mg, 0.880 mmol) in toluene (5 mL) were added Intermediate 20 (113 mg, 1.32 mmol), tripotassium phosphate (374 mg, 1.76 mmol) and tricyclohexylphosphine (49.4 mg, 0.176 mmol) and this mixture was degassed with nitrogen gas for 10 min. Palladium(II) acetate (19.7 mg, 0.088 mmol) was added to the reaction mixture and heated at 100° C. for 16 h. After the consumption of the starting material the reaction mixture was filtered through celite and washed with DCM (20 mL). The filtrate was concentrated under reduced pressure and the residue was purified by flash column chromatography (10 g silica, eluted with Pet-ether:EtOAc (100:0 to 60:40)). The desired fractions were combined and concentrated under reduced pressure to afford tert-butyl (tert-butoxycarbonyl)(6-(4-cyclopropyl-5-methyloxazol-2-yl)pyridin-3-yl)carbamate (150 mg, 40%) as a yellow gum.

LCMS (System 1, Method D): m/z 416 (M+H)⁺ (ESI⁺), Rₜ 3.21 min, 99%, UV active.

To a stirred solution of tert-butyl (tert-butoxycarbonyl)(6-(4-cyclopropyl-5-methyloxazol-2-yl)pyridin-3-yl)carbamate (220 mg, 0.529 mmol) in DCM (3 mL) at 0° C. was added 4M HCl in dioxane (5 mL, 20.0 mmol). The reaction mixture was stirred at RT for 3 h. After the consumption of the starting material the reaction mixture was concentrated under reduced pressure and the resulting residue was dried under a high vacuum to afford Intermediate 59 (150 mg, 96%) as a pale brown solid.

The data for Intermediate 59 are in Table 3.

### Route 17

### Typical Procedure for the Preparation of Substituted 6-(5-cyclopropyl-1H-pyrazol-3-yl)pyridin-3-amines, as exemplified by the preparation of Intermediate 62, 6-(5-cyclopropyl-1H-pyrazol-3-yl)pyridin-3-amine

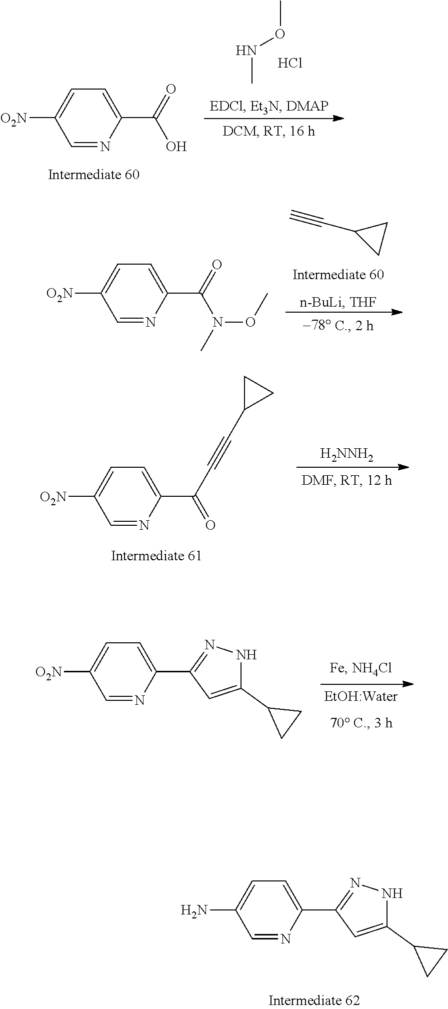

To a stirred solution of Intermediate 60 (500 mg, 2.97 mmol) in DCM (4 mL) at 0° C. were added N,O-dimethylhydroxylamine hydrochloride (377 mg, 3.87 mmol), EDCl (912 mg, 4.76 mmol), Et₃N (0.95 mL, 6.84 mmol) and DMAP (109 mg, 0.892 mmol). The reaction mixture was stirred at RT for 16 h. After the consumption of the starting material the reaction mixture was diluted with water (10 mL) and extracted with DCM (3×15 mL). The combined organic layers were washed with brine (10 mL), dried over Na₂SO₄ and concentrated under reduced pressure. The residue was purified by flash column chromatography (eluted with Pet-ether:EtOAc (90:10 to 20:80)). The desired fractions were combined and concentrated under reduced pressure to afford N-methoxy-N-methyl-5-nitropicolinamide (490 mg, 78%) as a brown solid.

LCMS (System 1, Method G): m/z 212 (M+H)⁺ (ESI⁺), Rₜ 1.36 min, 99%, UV active.

To a stirred solution of Intermediate 26 (81 mg, 1.23 mmol) in THF (1 mL) at −78° C. was added n-BuLi (1.6 M in hexanes) (0.740 mL, 1.18 mmol) and stirred for 15 minutes. N-methoxy-N-methyl-5-nitropicolinamide (200 mg, 0.947 mmol) in THF (1 mL) was added dropwise to the reaction mixture at −78° C. The reaction mixture was stirred at RT for 2 h. After the consumption of the starting material the reaction mixture was quenched with aqueous NaHCO₃ (10 mL) and extracted with EtOAc (3×15 mL). The combined organic layers were washed with brine (10 mL), dried over Na₂SO₄, concentrated under reduced pressure to afford Intermediate 61 (184 mg, 82%) as brown solid.

The data for Intermediate 61 are in Table 3.

To a stirred solution of Intermediate 61 (0.800 g, 3.70 mmol) in DMF (6 mL) at RT was added hydrazine (0.116 mL, 3.70 mmol) at 0° C. The resultant reaction mixture was stirred at RT for 12 h. After the consumption of the starting material the reaction mixture was poured in ice-cold water (100 mL). The resulting solid was collected by filtration, rinsed with water (100 mL) and dried under vacuum to afford 2-(5-cyclopropyl-1H-pyrazol-3-yl)-5-nitropyridine (600 mg, 70%) as a yellow solid.

LCMS (System 1, Method D): m/z 231 (M+H)⁺ (ESI⁺), Rₜ 2.39 min, 99%, UV active.

To a stirred solution of 2-(5-cyclopropyl-1H-pyrazol-3-yl)-5-nitropyridine (300 mg, 1.30 mmol) in EtOH (3 mL) and water (1.28 mL) at RT were added iron (218 mg, 3.91 mmol) and ammonium chloride (209 mg, 3.91 mmol). Resultant reaction mixture was stirred at 70° C. for 3 h. After the consumption of the starting material the reaction mixture was cooled to RT, filtered through celite and washed with DCM (20 mL). The filtrate was concentrated under reduced pressure then the residue obtained was diluted in DCM (100 mL) and washed with water (70 mL). The aqueous layer was again extracted with DCM (50 mL). The combined organic layers were washed with saturated brine solution (50 mL), dried over anhydrous Na₂SO₄ and concentrated under reduced pressure to afford Intermediate 62 (230 mg, 87%) as a yellow solid.

The data for Intermediate 62 are in Table 3.

### Route 18

### Typical Procedure for the Preparation of Substituted 6-(5-cyclopropyl-1H-pyrazol-3-yl)pyridin-3-amines, as Exemplified by the Preparation of Intermediate 63, 6-(5-cyclopropyl-1-methyl-1H-pyrazol-3-yl)pyridin-3-amine

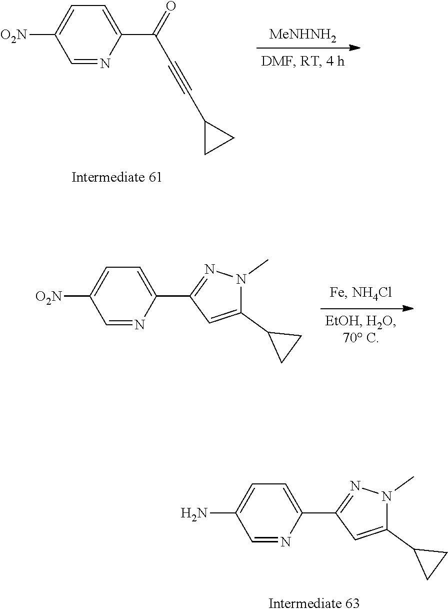 [SMILES: "MeNHNH2" ; O=C(C#CC1CC1)c1ccc([N+](=O)[O-])cn1 ; Cn1nc(-c2ccc([N+](=O)[O-])cn2)cc1C1CC1 ; Cn1nc(-c2ccc(N)cn2)cc1C1CC1]

To a stirred solution of Intermediate 61 (180 mg, 0.833 mmol) in DMF (3 mL) was added methyl hydrazine (46 mg, 0.999 mmol). The reaction mixture was stirred at RT for 4 h. After the consumption of the starting material the reaction mixture was added to ice-cooled water slowly with stirring. The resulting solid was collected by filteration, washed with water (5 mL), and dried under high vacuum to afford 2-(5-cyclopropyl-1-methyl-1H-pyrazol-3-yl)-5-nitropyridine (136 mg, 63%) as brown solid.

LCMS (System 1, Method C): m/z 245 (M+H)⁺ (ESI⁺), Rₜ 2.16 min, 94%, UV active.

To a stirred solution of 2-(5-cyclopropyl-1-methyl-1H-pyrazol-3-yl)-5-nitropyridine (140 mg, 0.573 mmol) in EtOH (5 mL) and water (2 mL) were added iron (160 mg, 2.87 mmol) and ammonium chloride (153 mg, 2.87 mmol) and heated at 70° C. for 2 h. After the consumption of the starting material the reaction mixture was concentrated under pressure. The residue was filtered through celite using 9:1 DCM:MeOH (10 mL). The filtrate was washed with water (10 mL), brine (20 mL), dried over Na₂SO₄ and then concentrated under reduced pressure to afford Intermediate 63 (127 mg, 98%) as a brown solid.

The data for Intermediate 63 are in Table 3.

### Route 19

### Typical Procedure for the Preparation of Substituted 2-bromo-5-(5-cyclopropyl-1-methyl-1H-pyrazol-3-yl)pyridines, as Exemplified by the Preparation of Intermediate 65, 2-bromo-5-(5-cyclopropyl-1-methyl-1-pyrazol-3-yl)pyridine

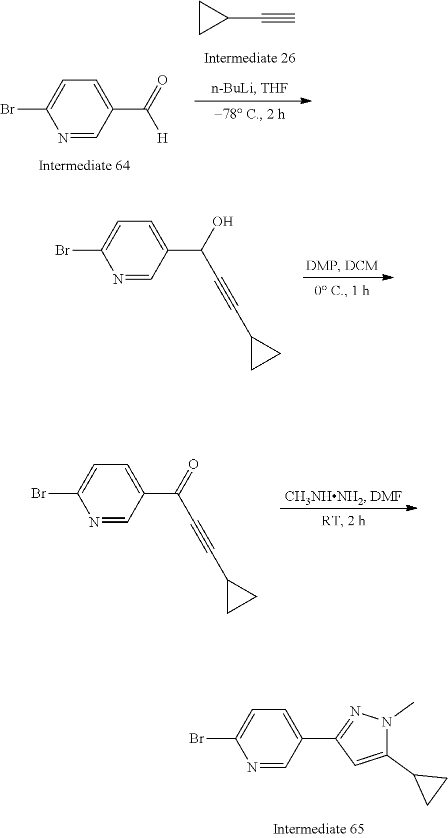

To a stirred solution of Intermediate 26 (178 mg, 2.69 mmol) in THF (10 mL) at −78° C. was added n-butyllithium (1.84 ml, 2.96 mmol) dropwise and stirred at −78° C. for 15 min. Intermediate 64 (500 mg, 2.69 mmol) in THF (5 mL) was added to the reaction mixture and stirred at RT for 2 h. After the consumption of the starting material the reaction mixture was cooled to −40° C., quenched with sat NaHCO₃ solution and extracted with EtOAc (2×10 mL). The combined organic extracts were washed with brine (10 mL), dried over Na₂SO₄, filtered and concentrated under reduced pressure. The residue was purified by flash column chromatography (25 g silica, eluted with Pet-ether:EtOAc (85:15 to 80:20)). The desired fractions were combined and concentrated under reduced pressure to afford 1-(6-bromopyridin-3-yl)-3-cyclopropylprop-2-yn-1-ol (500 mg, 73%) as a colorless liquid.

LCMS (System 1, Method A): m/z 251 (M+H)⁺ (ESI⁺), Rₜ 2.29 min, 99%, UV active.

To a stirred solution of 1-(6-bromopyridin-3-yl)-3-cyclopropylprop-2-yn-1-ol (500 mg, 1.98 mmol) in DCM (10 mL) at 0° C. was added Dess-Martin periodinane (1.26 g, 2.97 mmol) and stirred at 0° C. for 1 h. After the consumption of the starting material the reaction mixture was filtered through celite and washed with EtOAc (20 mL). The filtrate was washed with sat NaHCO₃ solution (10 mL), brine (10 mL), dried over Na₂SO₄, filtered and concentrated under reduced pressure. The residue was purified by flash column chromatography (25 g silica, eluted with Pet-ether:EtOAc (80:20 to 70:30)). The desired fractions were combined and concentrated under reduced pressure to afford 1-(6-bromopyridin-3-yl)-3-cyclopropylprop-2-yn-1-one (410 mg, 82%) as a white solid.

LCMS (System 1, Method A): m/z 250 (M+H)⁺ (ESI⁺), Rₜ 2.31 min, 98%, UV active.

To a stirred solution of 1-(6-bromopyridin-3-yl)-3-cyclopropylprop-2-yn-1-one (410 mg, 1.63 mmol) in DMF (5 mL) at RT was added methylhydrazine (91 mg, 1.96 mmol) and this was stirred at RT for 2 h. After the consumption of the starting material the reaction mixture was poured into crushed ice. The resulting solid obtained collected by filtration and dried under vacuum to afford Intermediate 65 (320 mg, 69%) as an off white solid.

The data for Intermediate 65 are in Table 3.

### Route 20

### Typical Procedure for the Preparation of Primary Amides, as Exemplified by the Preparation of Intermediate 66, 3-((1,1-dioxidothiomorpholino)methyl)benzamide

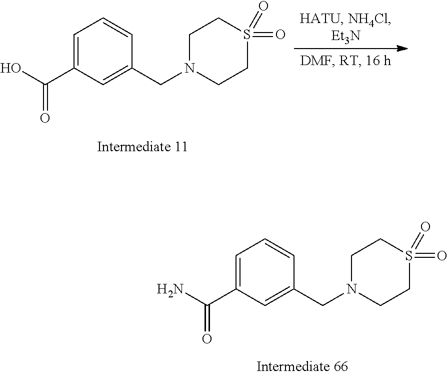 [SMILES: O=C(O)c1cccc(CN2CCS(=O)(=O)CC2)c1 ; NC(=O)c1cccc(CN2CCS(=O)(=O)CC2)c1]

To a stirred solution of Intermediate 11 (1.00 g, 3.71 mmol) in DMF (10 mL) at 0° C. were added Et₃N (1.55 mL, 11.1 mmol), ammonium chloride (0.99 g, 18.5 mmol) and HATU (2.11 g, 5.57 mmol). The reaction mixture was stirred at RT for 16 h. After the consumption of the starting material the reaction mixture was diluted with water (30 mL) and extracted with 10% MeOH in DCM (3×30 mL). The combined organic layers were washed with brine solution (10 mL), dried over Na₂SO₄ and concentrated under reduced pressure. The residue was purified by flash column chromatography (25 g silica, eluted with DCM:MeOH (100:0 to 90:10)). The desired fractions were combined and concentrated under reduced pressure to afford Intermediate 66 (450 mg, 45%) as an off white solid.

The data for Intermediate 66 are in Table 3.

### General Synthetic Procedures:

### Route A

### Typical Procedure for the Preparation of Amides as Exemplified by the Preparation of Example 1-1, N-[4-(3-cyclopropyl-1H-1,2,4-triazol-5-yl)phenyl]-3-(thiomorpholin-4-ylmethyl)benzamide

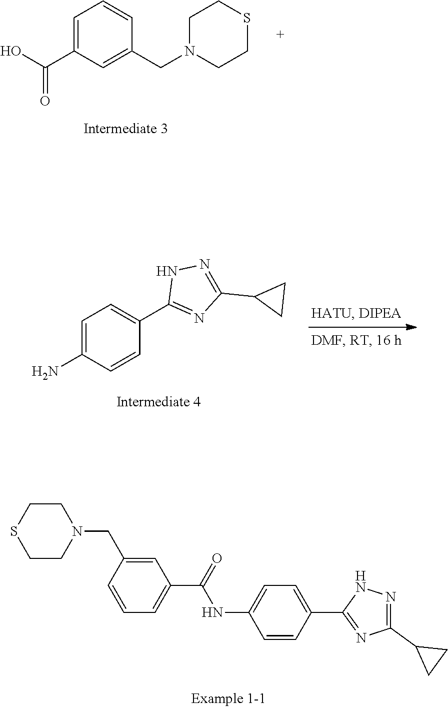 [SMILES: O=C(O)c1cccc(CN2CCSCC2)c1 ; Nc1ccc(-c2nc(C3CC3)n[nH]2)cc1 ; O=C(Nc1ccc(-c2nc(C3CC3)n[nH]2)cc1)c1cccc(CN2CCSCC2)c1]

To a stirred solution of Intermediate 3 (90 mg, 0.38 mmol) in DMF (3 mL) were added DIPEA (0.2 mL, 1.14 mmol), HATU (173 mg, 0.46 mmol) and Intermediate 4 (75 mg, 0.38 mmol). The reaction mixture was stirred at RT for 16 h. After the consumption of the starting material the reaction mixture was extracted with EtOAc (10 mL), washed with brine solution, dried over Na₂SO₄ and concentrated under reduced pressure. The residue was purified by flash column chromatography (25 g silica, eluted with Pet-ether:EtOAc (80:20 to 10:90)). The desired fractions were combined and concentrated under reduced pressure to afford Example 1-1 (57 mg, 36%) as an off white solid.

The data for Example 1-1 are in Table 5.

### Route B

### Typical Procedure for the Preparation of Amines as Exemplified by the Preparation of Example 1-3, N-[4-(3-cyclopropyl-1H-1,2,4-triazol-5-yl)phenyl]-3-[(2,2-dimethyl-1,1-dioxo-1,4-thiazinan-4-yl)methyl]benzamide

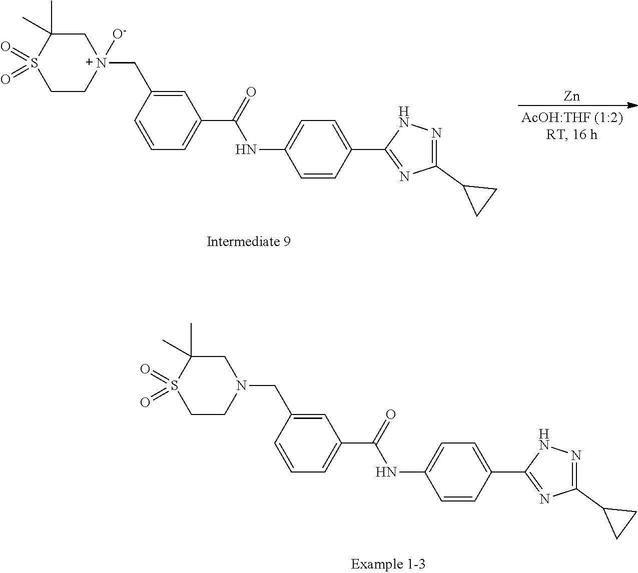

To the stirred solution of Intermediate 9 (125 mg, 0.252 mmol) in THF:acetic acid (2:1) (6 mL) was added zinc (49.5 mg, 0.757 mmol). The resultant reaction mixture was stirred at RT for 16 h. After the consumption of the starting material the reaction mixture was filtered through celite and the filtrate was concentrated under reduced pressure. The residue was purified by Purification Method C. The desired fractions were combined and concentrated under reduced pressure. The residue was dissolved in DCM (5 mL), washed with water (5 mL), dried over Na₂SO₄, evaporated and lyophilized to afford Example 1-3 (12 mg, 10%) as an off white solid.

The data for Example 1-3 are in Table 5.

### Route C

### Typical Procedure for the Preparation of Amides as Exemplified by the Preparation of Example 3-1, 3-[(1,1-dioxo-1,4-thiazinan-4-yl)methyl]-N-[4-(3-propan-2-yl-1H-1,2,4-triazol-5-yl)phenyl]benzamide HCl salt

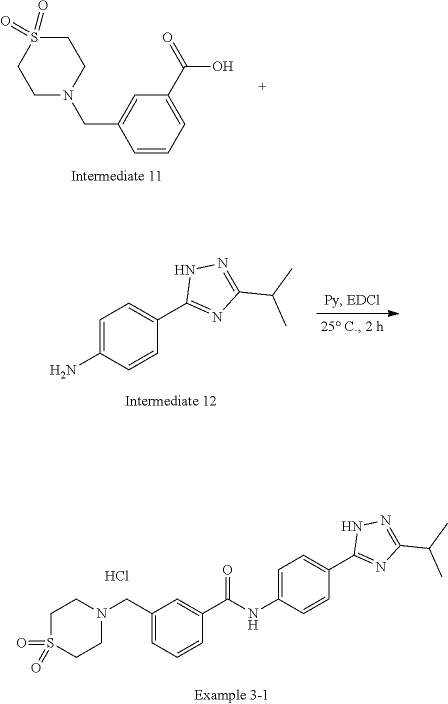 [SMILES: O=C(O)c1cccc(CN2CCS(=O)(=O)CC2)c1 ; CC(C)c1n[nH]c(-c2ccc(N)cc2)n1 ; CC(C)c1n[nH]c(-c2ccc(NC(=O)c3cccc(CN4CCS(=O)(=O)CC4)c3)cc2)n1 ; Cl]

A mixture of Intermediate 11 (200 mg, 0.742 mmol), Intermediate 12 (150 mg, 0.742 mmol) and EDCl (213 mg, 1.11 mmol) in pyridine (2 mL) was stirred at 20° C. for 12 h. The reaction mixture was filtered to give a residue which was purified by Purification Method D to afford Example 3-1 (87 mg, 24% yield) as a white solid.

The data for Example 3-1 are in Table 5.

### Route D

### Typical Procedure for the Preparation of Amides as Exemplified by the Preparation of Example 4-20, 4-chloro-N-[4-(5-cyclopropyl-1H-pyrazol-3-yl)phenyl]-3-[(1,1-dioxo-1,4-thiazinan-4-yl)methyl]benzamide

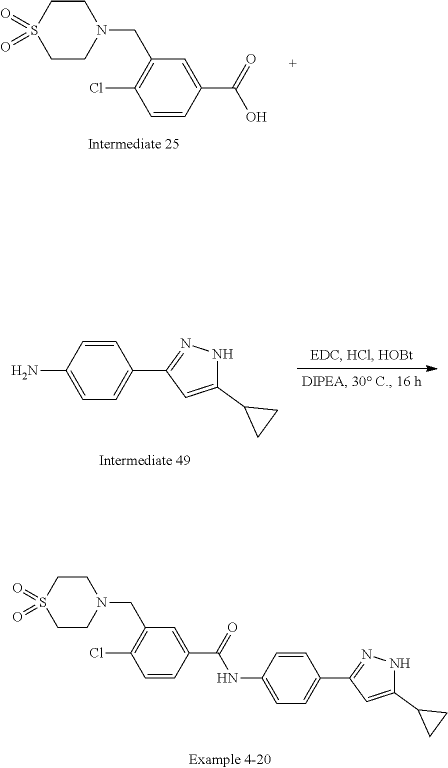 [SMILES: O=C(O)c1ccc(Cl)c(CN2CCS(=O)(=O)CC2)c1 ; Nc1ccc(-c2cc(C3CC3)[nH]n2)cc1 ; O=C(Nc1ccc(-c2cc(C3CC3)[nH]n2)cc1)c1ccc(Cl)c(CN2CCS(=O)(=O)CC2)c1]

To a stirred solution of Intermediate 25 (100 mg, 0.329 mmol) in DCM (10 mL) was added EDC·HCl (95 mg, 0.494 mmol), HOBt (101 mg, 0.658 mmol), DIPEA (0.115 mL, 0.658 mmol) and Intermediate 49 (79 mg, 0.395 mmol) under a nitrogen atmosphere. The resultant reaction mixture was stirred at 30° C. temperature for 16 h. After the consumption of the starting material the reaction mixture was diluted with water (20 mL) and extracted with DCM (3×30 mL). The combined organic extracts were washed with saturated brine solution (20 mL), dried over anhydrous Na₂SO₄, filtered and concentrated under reduced pressure. The residue was purified by Purification Method C. The desired fractions were combined and concentrated under reduced pressure and lyophilized to afford Example 4-20 (49 mg, 30%) as a white solid.

The data for Example 4-20 are in Table 5.

### Route E

### Typical Procedure for the Preparation of Amides as Exemplified by the Preparation of Example 4-22, 4-chloro-N-[4-(5-cyclopropyl-4-methyl-1H-pyrazol-3-yl)phenyl]-3-[(1,1-dioxo-1,4-thiazinan-4-yl)methyl]benzamide

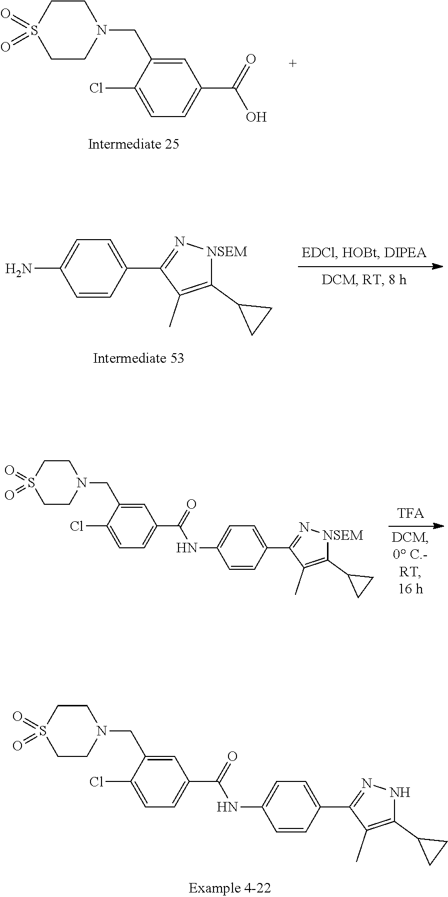 [SMILES: O=C(O)c1ccc(Cl)c(CN2CCS(=O)(=O)CC2)c1 ; Cc1c(-c2ccc(N)cc2)nn(COCC[Si](C)(C)C)c1C1CC1 ; Cc1c(-c2ccc(NC(=O)c3ccc(Cl)c(CN4CCS(=O)(=O)CC4)c3)cc2)nn(COCC[Si](C)(C)C)c1C1CC1 ; Cc1c(-c2ccc(NC(=O)c3ccc(Cl)c(CN4CCS(=O)(=O)CC4)c3)cc2)n[nH]c1C1CC1]

To a stirred solution of Intermediate 25 (140 mg, 0.461 mmol) in DCM (4 mL) was added EDCl (133 mg, 0.691 mmol), HOBt (141 mg, 0.922 mmol), DIPEA (0.161 mL, 0.922 mmol) and Intermediate 53 (158 mg, 0.461 mmol) under a nitrogen atmosphere. The reaction mixture was stirred at RT temperature for 8 h. After the consumption of the starting material the reaction mixture was diluted with water (15 mL) and extracted with DCM (3×20 mL). The combined organic extracts were washed with brine (15 mL), dried over anhydrous Na₂SO₄, filtered and concentrated under reduced. The residue was purified by flash column chromatography (25 g silica, eluted with Pet-ether:EtOAc (100:0 to 40:60)). The desired fractions were combined and concentrated under reduced pressure to afford 4-chloro-N-(4-(5-cyclopropyl-4-methyl-1-((2-(trimethylsilyl)ethoxy)methyl)-1H-pyrazol-3-yl)phenyl)-3-((1,1-dioxidothiomorpholino)methyl)benzamide (160 mg, 43%) as a light brown gum.

LCMS (System 1, Method D)): m/z 629 (M+H)⁺ (ESI⁺), Rₜ 3.56 min, 78%, UV active.

To a stirred solution of 4-chloro-N-(4-(5-cyclopropyl-4-methyl-1-((2-(trimethylsilyl)ethoxy)methyl)-1H-pyrazol-3-yl)phenyl)-3-((1,1-dioxidothiomorpholino)methyl)benzamide (160 mg, 0.254 mmol) in DCM (3 mL) at 0° C. was added TFA (19.5 μL, 0.254 mmol). The resultant reaction mixture was stirred at RT for 16 h. After the consumption of the starting material the reaction mixture was concentrated under reduced pressure. The residue was purified by Purification Method F. The desired fractions were combined and concentrated under reduced pressure then basified by 10% NaHCO₃ (6 mL). The resulting precipitate was filtered to afford Example 4-22 (32 mg, 25%) as an off white solid.

The data for Example 4-22 are in Table 5.

### Route F

### Typical Procedure for the Preparation of Alkyl Triazoles as Exemplified by the Preparation of Example 4-27, N-[4-(5-cyclopropyl-1-methyl-1,2,4-triazol-3-yl)phenyl]-3-[(1,1-dioxo-1,4-thiazinan-4-yl)methyl]benzamide HCl salt

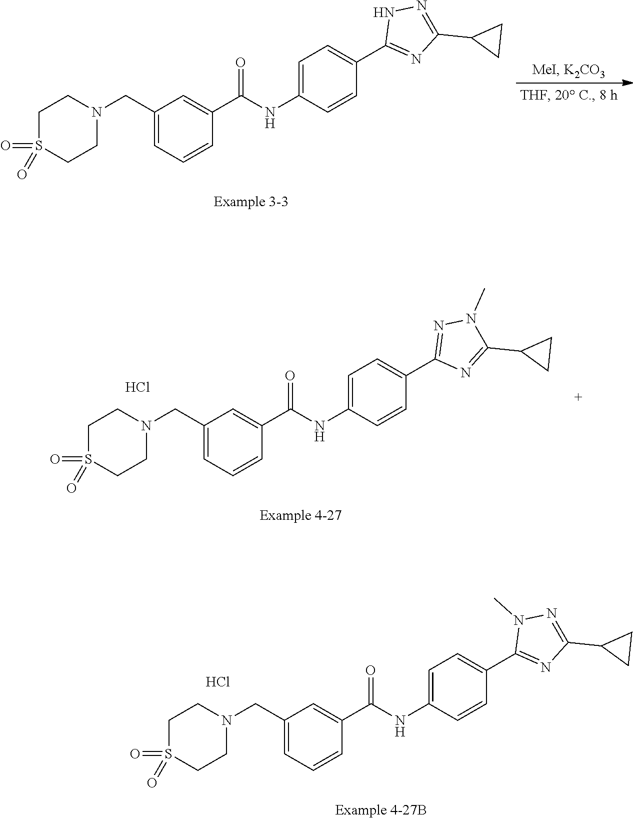 [SMILES: O=C(Nc1ccc(-c2nc(C3CC3)n[nH]2)cc1)c1cccc(CN2CCS(=O)(=O)CC2)c1 ; Cn1nc(-c2ccc(NC(=O)c3cccc(CN4CCS(=O)(=O)CC4)c3)cc2)nc1C1CC1 ; Cl ; Cn1nc(C2CC2)nc1-c1ccc(NC(=O)c2cccc(CN3CCS(=O)(=O)CC3)c2)cc1 ; Cl]

To a stirred solution of Example 3-3 (100 mg, 0.221 mmol) and K₂CO₃ (61.2 mg, 0.442 mmol) in THF (5 mL) was added dropwise iodomethane (137 μL, 2.21 mmol) at 0° C. After the addition the mixture was stirred at 20° C. for 8 h. The reaction mixture was filtered and the filtrate was concentrated under reduced pressure. The residue was purified by Purification Method D to afford Example 4-27 (18.2 mg, 16% yield) and Example 4-27B (5.11 mg, 4% yield) as a white solid.

The data for Example 4-27 are in Table 5.

### Route G

### Typical Procedure for the Preparation of Pyridyl Amides as Exemplified by the Preparation of Example 5-1, N-[6-(4-bromo-5-methyl-1,3-oxazol-2-yl)pyridin-3-yl]-4-chloro-3-[(1,1-dioxo-1,4-thiazinan-4-yl)methyl]benzamide

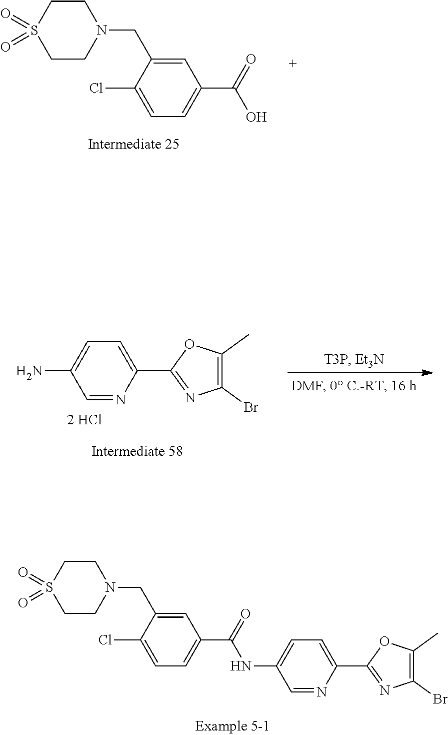 [SMILES: O=C(O)c1ccc(Cl)c(CN2CCS(=O)(=O)CC2)c1 ; Cc1oc(-c2ccc(N)cn2)nc1Br ; Cc1oc(-c2ccc(NC(=O)c3ccc(Cl)c(CN4CCS(=O)(=O)CC4)c3)cn2)nc1Br]

To a stirred solution of Intermediate 58 (50 mg, 0.153 mmol) in DMF (1 mL) were added Intermediate 25 (55.7 mg, 0.183 mmol) and Et₃N (0.064 mL, 0.459 mmol) at RT. The reaction mixture was cooled to 0° C., and T3P in EtOAc (0.117 mL, 0.199 mmol) was added. The resultant reaction mixture was stirred at RT for 16 h. After the consumption of the starting material the reaction mixture was basified with 10% NaHCO₃ solution (10 mL) and extracted with EtOAc (2×20 mL). The combined organic layers were dried over anhydrous Na₂SO₄, filtered, and concentrated under reduced pressure. The residue was purified by Purification Method F. The desired fractions were combined were concentrated under reduced pressure. The desired fractions were combined and concentrated under reduced pressure. The residue was basified with 10% NaHCO₃ solution (5 mL) and extracted with EtOAc (2×10 mL). The combined organic layers were dried over anhydrous Na₂SO₄, filtered, concentrated under reduced pressure, and lyophilized to afford Example 5-1 (19.1 mg, 23%) as a white solid.

The data for Example 5-1 are in Table 5.

### Route H

### Typical Procedure for the Preparation of Pyridyl Amides as Exemplified by the Preparation of Example 5-5, N-[5-(5-cyclopropyl-1-methylpyrazol-3-yl)pyridin-2-yl]-3-[(1,1-dioxo-1,4-thiazinan-4-yl)methyl]benzamide

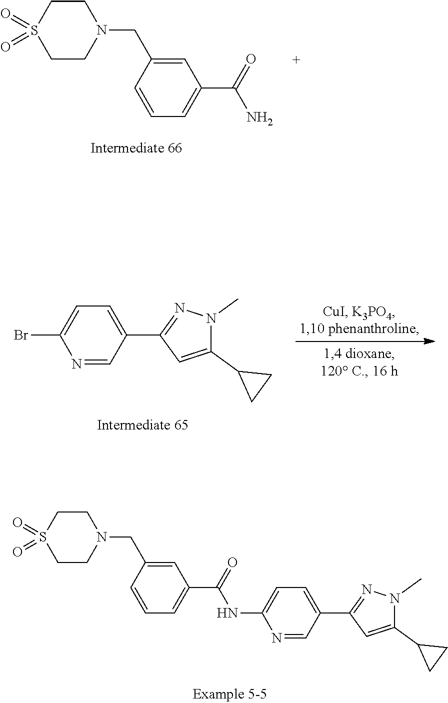 [SMILES: NC(=O)c1cccc(CN2CCS(=O)(=O)CC2)c1 ; Cn1nc(-c2ccc(Br)nc2)cc1C1CC1 ; Cn1nc(-c2ccc(NC(=O)c3cccc(CN4CCS(=O)(=O)CC4)c3)nc2)cc1C1CC1]

To a degassed solution of Intermediate 66 (100 mg, 0.373 mmol) and Intermediate 65 (100 mg, 0.40 mmol) in 1,4 dioxane (10 mL) was added K₃PO₄ (166 mg, 0.783 mmol), copper(I) iodide (7.10 mg, 0.037 mmol), and 1,10-phenanthroline (13.4 mg, 0.075 mmol). The reaction mixture was heated at 120° C. for 16 h. After the consumption of the starting material the reaction mixture was filtered through celite and washed with EtOAc (10 mL). The filtrate was concentrated under reduced pressure, and the residue was purified by Purification Method C. The desired fractions were combined and concentrated under reduced pressure. The residue was dissolved in EtOAc (10 mL) and washed with water (3 mL), dried over Na₂SO₄, concentrated under reduced pressure and lyophilized to afford Example 5-5 (15 mg 8%) as an off white solid.

The data for Example 5-5 are in Table 5.

TABLE 3
NMR and LCMS properties and the methods used to prepare and purify or
purchase intermediates 1-66.
Table 3

| Intermediate | | Synthetic | Intermedi- | |
|---|---|---|---|---|
| Number | Name | Route | ates Used | Data |
| 1 | Thiomorpholine | — | — | Commercially available, |
| | | | | CAS: 123-90-0 |
| 2 | Methyl 3- | — | — | Commercially available, |
| | (bromomethyl)benzoate | | | CAS: 1129-28-8 |
| 3 | 3- | 1 | 1 and 2 | LCMS (System 1, Method B): m/z |
| | (Thiomorpholinomethyl)benzoic | | | 238 (M + H)⁺ (ESI⁺), at 1.36 min, |
| | acid | | | UV active |
| 4 | 4-(3-Cyclopropyl-1H-1,2,4- | — | — | Commercially available, |
| | triazol-5-yl)aniline | | | CAS: 1240528-81-7 |
| 5 | 2-Methylthiomorpholine-1,1- | — | — | Commercially available, |
| | dioxide hydrochloride | | | CAS: 1797554-25-6 |
| 6 | 3-((2-Methyl-1,1- | 1 | 2 and 5 | LCMS (System 1, Method C): m/z |
| | dioxidothiomorpholino)meth- | | | 284 (M + H)⁺ (ESI⁺), at 1.09 min, |
| | yl)benzoic acid | | | UV active |
| 7 | 2,2-Dimethylthiomorpholine | — | — | Commercially available, |
| | | | | CAS: 3970-89-6 |
| 8 | 3-((2,2- | 1 | 2 and 7 | LCMS (System 1, Method C): m/z |
| | Dimethylthiomorpholino) meth- | | | 266 (M + H)⁺ (ESI⁺), at 0.77 min, |
| | yl)benzoic acid | | | UV active |
| 9 | 4-(3-((4-(3-Cyclopropyl-1H-1,2,4- | 2 | 4 and 8 | LCMS (System 1, Method A): m/z |
| | triazol-5- | | | 496 (M + H)⁺ (ESI⁺), at 1.77 min, |
| | yl)phenyl)carbamoyl)benzyl)-2,2- | | | UV active |
| | dimethylthiomorpholine 4-oxide | | | |
| | 1,1-dioxide | | | |
| 10 | Thiomorpholine, 1,1-dioxide | — | — | Commercially available, |
| | | | | CAS: 39093-93-1 |
| 11 | 3-((1,1- | 1 | 2 and 10 | ¹H NMR (400 MHZ, DMSO-d₆) δ |
| | Dioxidothiomorpholino) meth- | | | 7.92 (s, 1H), 7.86 (d, J = 7.7 Hz, |
| | yl)benzoic acid | | | 1H), 7.59 (d, J = 7.6 Hz, 1H), 7.52- |
| | | | | 7.42 (m, 1H), 3.74 (s, 2H), 3.16- |
| | | | | 3.06 (m, 4H), 2.88 (br dd, J = 3.6, |
| | | | | 6.1 Hz, 4H) |
| 12 | 4-[3-(1-Methylethyl)-1 H-1,2,4- | — | — | Commercially available, |
| | triazol-5-yl]benzenamine | | | CAS: 1216224-30-4 |
| 13 | 4-Nitrobenzonitrile | — | — | Commercially available, |
| | | | | CAS: 619-72-7 |
| 14 | N- | — | — | Commercially available, |
| | Hydroxycyclobutanecarboximida- | | | CAS: 99623-08-2 |
| | mide | | | |
| 15 | 4-(3-Cyclobutyl-1 H-1,2,4-triazol- | 3 | 13 and 14 | ¹H NMR (400 MHZ, DMSO-d₆) δ |
| | 5-yl)aniline | | | 7.67-7.79 (m, 2H), 6.57-6.74 |
| | | | | (m, 2H), 3.66-3.81 (m, 1H), 2.30- |
| | | | | 2.48 (m, 4H), 1.86-2.12 (m, 2H) |
| 16 | (Z)-N-Hydroxy-2- | — | — | Commercially available, |
| | phenylacetimidamide | | | CAS: 875898-38-7 |
| 17 | 4-(3-Benzyl-1H-1,2,4-triazol-5- | 3 | 13 and 16 | ¹H NMR ((400 MHZ, DMSO-d₆) δ |
| | yl) aniline | | | 7.93 (d, J = 8.38 Hz, 2H), 7.28- |
| | | | | 7.38 (m, 4H), 7.20-7.28 (m, 1H), |
| | | | | 6.96-7.12 (m, 2H), 4.12 - 4.23 (m, |
| | | | | 2H) |
| 18 | 4-Nitrobenzoyl chloride | — | — | Commercially available, |
| | | | | CAS: 122-04-3 |
| 19 | Propargylamine | — | — | Commercially available, |
| | | | | CAS: 2450-71-7 |
| 20 | Cyclopropylboronic acid | — | — | Commercially available, |
| | | | | CAS: 411235-57-9 |
| 21 | 4-(4-Cyclopropyl-5- | 4 | 18, 19 and | LCMS (System 1, Method A): m/z |
| | methyloxazol-2-yl)aniline | | 20 | 215 (M + H)⁺ (ESI⁺), at 1.58 min, |
| | | | | UV active |
| 22 | Ethyl 3-(chloromethyl)-4- | — | — | Commercially available, |
| | fluorobenzoate | | | CAS: 1379355-85-7 |
| 23 | 3-((1,1- | 1 | 10 and 22 | LCMS (System 1, Method D): m/z |
| | Dioxidothiomorpholino)methyl)- | | | 288 (M + H)⁺ (ESI⁺), at 1.74 min, |
| | 4-fluorobenzoic acid | | | UV active |
| 24 | Methyl 3-(bromomethyl)-4- | — | — | Commercially available, |
| | chlorobenzoate | | | CAS: 220464-68-6 |
| 25 | 4-Chloro-3-((1,1- | 1 | 10 and 24 | LCMS (System 1, Method C): m/z |
| | dioxidothiomorpholino)meth- | | | 304 (M + H)⁺ (ESI⁺), at 1.58 min, |
| | yl)benzoic acid | | | UV active |
| 26 | Cyclopropylacetylene | — | — | Commercially available, |
| | | | | CAS: 6746-94-7 |
| 27 | 4-(3-Cyclopropylisoxazol-5- | 5 | 18 and 26 | LCMS (System 1, Method A): m/z |
| | yl) aniline | | | 201 (M + H)⁺ (ESI⁺), at 1.91 min, |
| | | | | UV active |
| 28 | 4-Nitrobenzaldehyde | — | — | Commercially available, |
| | | | | CAS: 555-16-8 |
| 29 | 4-(5-Cyclopropylisoxazol-3- | 6 | 26 and 28 | LCMS (System 1, Method D): m/z |
| | yl) aniline | | | 201 (M + H)⁺ (ESI⁺), at 1.92 min, |
| | | | | UV active |
| 30 | 4-Nitrobenzoic acid | — | — | Commercially available, |
| | | | | CAS: 62-23-7 |
| 31 | Cyclopropanecarbohydrazide | — | — | Commercially available, |
| | | | | CAS: 6952-93-8 |
| 32 | 4-(5-Cyclopropyl-1,3,4- | 7 | 30 and 31 | LCMS (System 2, Method E): m/z |
| | oxadiazol-2-yl)aniline | | | 202 (M + H)⁺ (ESI⁺), at 0.73 min, |
| | | | | UV active |
| 33 | N- | — | — | Commercially available, |
| | Hydroxycyclopropanecarboximid- | | | CAS: 51285-13-3 |
| | amide | | | |
| 34 | 4-(3-Cyclopropyl-1,2,4- | 8 | 30 and 33 | ¹H NMR (400 MHZ, DMSO-d₆) δ |
| | oxadiazol-5-yl)aniline | | | 7.68 (d, J = 8.68 Hz, 2H), 6.61- |
| | | | | 6.68 (m, 2H), 6.10 (s, 2H), 2.02- |
| | | | | 2.15 (m, 1H), 0.89-1.09 (m, 4H) |
| 35 | Cyclopropanecarboxylic acid | — | — | Commercially available, |
| | | | | CAS: 1759-53-1 |
| 36 | 4-(5-Cyclopropyl-1,2,4- | 9 | 13 and 35 | ¹H NMR (400 MHZ, Chloroform-d) |
| | oxadiazol-3-yl)aniline | | | δ 7.72-7.81 (m, 2H), 7.19 (s, 1H), |
| | | | | 6.57-6.71 (m, 2H), 3.67-4.08 (m, |
| | | | | 2H), 2.15 (tt, J = 8.19, 5.01 Hz, 1H), |
| | | | | 1.18-1.24 (m, 2H), 1.11-1.18 (m, |
| | | | | 2H) |
| 37 | Cyclobutanecarboxylic acid | — | — | Commercially available, |
| | | | | CAS: 3721-95-7 |
| 38 | 4-(5-Cyclobutyl-1,2,4-oxadiazol- | 9 | 13 and 37 | ¹H NMR (400 MHZ, Methanol-d₄) δ |
| | 3-yl)aniline | | | 7.86-7.62 (m, 2H), 6.85-6.62 |
| | | | | (m, 2H), 3.93-3.75 (m, 1H), 2.57- |
| | | | | 2.40 (m, 4H), 2.27-2.12 (m, 1H), |
| | | | | 2.12-2.02 (m, 1H) |
| 39 | Cyclopentanecarboxylic acid | — | — | Commercially available, |
| | | | | CAS: 3400-45-1 |
| 40 | 4-(5-Cyclopentyl-1,2,4- | 9 | 13 and 39 | ¹H NMR (400 MHZ, Chloroform-d) |
| | oxadiazol-3-yl)aniline | | | δ 7.9-7.9 (m, 2H), 6.74 (d, J = 8.6 |
| | | | | Hz, 2H), 3.38 (quin, J = 8.0 Hz, 1H), |
| | | | | 2.1-2.2 (m, 2H), 1.9-2.1 (m, 2H), 1.8- |
| | | | | 1.9 (m, 2H), 1.7-1.8 (m, 2H) |
| 41 | 3-(4-Nitrophenyl)-5- | — | — | Commercially available, |
| | (phenylmethyl)-1,2,4-oxadiazole | | | CAS: 431908-57-5 |
| 42 | 4-(5-Benzyl-1,2,4-oxadiazol-3- | 9 | 41 | ¹H NMR (400 MHZ, Chloroform-d) |
| | yl) aniline | | | δ 7.87 (dd, J = 1.9, 8.7 Hz, 2H), 7.3- |
| | | | | 7.4 (m, 4H), 7.3-7.3 (m, 1H), 6.73 |
| | | | | (dd, J = 2.0, 8.7 Hz, 2H), 4.27 (d, J = |
| | | | | 1.7 Hz, 2H), 3.9-4.0 (m, 2H) |
| 43 | p-Tolylacetic acid | — | — | Commercially available, |
| | | | | CAS: 622-47-9 |
| 44 | 4-(5-(4-Methylbenzyl)-1,2,4- | 9 | 13 and 43 | ¹H NMR (400 MHZ, Chloroform-d) |
| | oxadiazol-3-yl)aniline | | | δ 7.87 (dd, J = 1.8, 8.5 Hz, 2H), 7.26 |
| | | | | (br s, 2H), 7.16 (br d, J = 7.0 Hz, |
| | | | | 2H), 6.75 (br d, J = 8.0 Hz, 2H), 4.2- |
| | | | | 4.3 (m, 2H), 2.3-2.4 (m, 3H) |
| 45 | (4-Fluorophenyl)acetic acid | — | — | Commercially available, |
| | | | | CAS: 405-50-5 |
| 46 | 4-(5-(4-Fluorobenzyl)-1,2,4- | 9 | 13 and 45 | ¹H NMR (400 MHZ, Chloroform-d) |
| | oxadiazol-3-yl)aniline | | | δ 7.8-7.9 (m, 2H), 7.35 (br dd, J = |
| | | | | 5.3, 7.2 Hz, 2H), 7.0-7.1 (m, 2H), |
| | | | | 6.7-6.8 (m, 2H), 4.24 (s, 2H), 3.8-4.1 |
| | | | | (m, 2H) |
| 47 | (4-Chlorophenyl)acetic acid | — | — | Commercially available, |
| | | | | CAS: 1878-66-6 |
| 48 | 4-(5-(4-Chlorobenzyl)-1,2,4- | 9 | 13 and 47 | ¹H NMR (400 MHZ, Methanol-d₄) δ |
| | oxadiazol-3-yl)aniline | | | 7.73 (d, J = 8.4 Hz, 2H), 7.38 (s, |
| | | | | 4H), 6.72 (d, J = 8.5 Hz, 2H), 4.30 |
| | | | | (s, 2H) |
| 49 | 3-Cyclopropyl-1-(4- | 10 | 18 and 26 | ¹H NMR (400 MHZ, Chloroform-d) |
| | nitrophenyl)prop-2-yn-1-one | | | δ 8.36-8.33 (m, 2H), 8.29-8.26 (m, |
| | | | | 2H), 1.64-1.57 (m, 1H), 1.17-1.08 |
| | | | | (m, 4H) |
| 50 | 4-(3-Cyclopropyl-1H-pyrazol-5- | 10 | 49 | LCMS (System 1, Method G): m/z |
| | yl) aniline | | | 200 (M + H)⁺ (ESI⁺), at 1.75 min, |
| | | | | UV active |
| 51 | 3-Phenyl-1-propyne | — | — | Commercially available, |
| | | | | CAS: 10147-11-2 |
| 52 | 4-(3-Benzyl-1H-pyrazol-5- | 11 | 28 and 51 | LCMS (System 1, Method C): m/z |
| | yl) aniline | | | 250 (M + H)⁺ (ESI⁺), at 1.53 min, |
| | | | | UV active |
| 53 | 4-(5-Cyclopropyl-4-methyl-1-((2- | 12 | 49 | LCMS (System 1, Method D): m/z |
| | (trimethylsilyl)ethoxy)methyl)-1H- | | | 344 (M + H)⁺ (ESI⁺), at 3.19 and |
| | pyrazol-3-yl)aniline | | | 3.33 min, UV active |
| 54 | 4-(5-Cyclopropyl-1-methyl-1H- | 13 | 49 | LCMS (System 1, Method C): m/z |
| | pyrazol-3-yl)aniline | | | 214 (M + H)⁺ (ESI⁺), at 1.15 min, |
| | | | | UV active |
| 55 | 4-(5-Cyclopropyl-1,4-dimethyl- | 14 | 49 | LCMS (System 1, Method D): m/z |
| | 1 H-pyrazol-3-yl)aniline | | | 228 (M + H)⁺ (ESI⁺), at 2.16 min, |
| | | | | UV active |
| 56 | 5-Aminopicolinic acid | — | — | Commercially available, |
| | | | | CAS: 24242-20-4 |
| 57 | tert-Butyl (6-(4-bromo-5- | 15 | 19 and 56 | LCMS (System 1, Method D): m/z |
| | methyloxazol-2-yl)pyridin-3- | | | 454 (M + H)⁺ (ESI⁺), at 2.88 min, |
| | yl) (tert- | | | UV active |
| | butoxycarbonyl)carbamate | | | |
| 58 | 6-(4-Bromo-5-methyloxazol-2- | 15 | 57 | LCMS (System 1, Method D): m/z |
| | yl)pyridin-3-amine di- | | | 255(M + H)⁺ (ESI⁺), at 2.27 min, |
| | hydrochloride | | | UV active |
| 59 | 6-(4-Cyclopropyl-5- | 16 | 57 | LCMS (System 1, Method D): m/z |
| | methyloxazol-2-yl)pyridin-3- | | | 216 (M + H)⁺ (ESI⁺), at 2.16 min, |
| | amine di-hydrochloride | | | UV active |
| 60 | 5-Nitropicolinic acid | — | — | Commercially available, |
| | | | | CAS: 30651-24-2 |
| 61 | 3-Cyclopropyl-1-(5-nitropyridin- | 17 | 26 and 60 | LCMS (System 1, Method G): m/z |
| | 2-yl)prop-2-yn-1-one | | | 217 (M + H)⁺ (ESI⁺), at 1.98 min, |
| | | | | UV active |
| 62 | 6-(5-Cyclopropyl-1H-pyrazol-3- | 17 | 61 | LCMS (System 1, Method D): m/z |
| | yl)pyridin-3-amine | | | 201 (M + H)⁺ (ESI⁺), at 1.30 min, |
| | | | | UV active |
| 63 | 6-(5-Cyclopropyl-1-methyl-1H- | 18 | 61 | LCMS (System 1, Method D): m/z |
| | pyrazol-3-yl)pyridin-3-amine | | | 215 (M + H)⁺ (ESI⁺), at 1.15 min, |
| | | | | UV active |
| 64 | 6-Bromo-3- | — | — | Commercially available, |
| | pyridinecarboxaldehyde | | | CAS: 149806-06-4 |
| 65 | 2-Bromo-5-(5-cyclopropyl-1- | 19 | 26 and 64 | LCMS (System 1, Method A): m/z |
| | methyl-1 H-pyrazol-3-yl)pyridine | | | 251 (M + H)⁺ (ESI⁺), at 2.29 min, |
| | | | | UV active |
| 66 | 3-((1,1- | 20 | 11 | LCMS (System 1, Method H): m/z |
| | Dioxidothiomorpholino)meth- | | | 269 (M + H)⁺ (ESI⁺), at 1.25 min, |
| | yl)benzamide | | | UV active |

TABLE 4
Table 4 - NMR and LCMS properties and the methods used to prepare and purify compounds represented by Examples 1-1 to 5-5

| | | Synthetic | | | LCMS | |
|---|---|---|---|---|---|---|
| | | Method and | | | System | |
| Ex. | | Intermediates | Purification | | and | LCMS |
| No. | Name | Used | Method | ¹H NMR or Supplier with Product ID | Method | data |
| 1-1 | N-[4-(3-Cyclopropyl-1H-1,2,4- | A | A | ¹H NMR (400 MHz, DMSO-d₆) δ 13.88 (s, 0.3H), 13.62 (s, 0.7H), 10.44 (s, | System 1 | m/z 420 |
| | triazol-5-yl)phenyl]-3- | 3 and 4 | | 0.3H), 10.35 (s, 0.7H), 7.94-7.84 (m, 6H), 7.52-7.48 (m, 2H), 3.59 (s, | Method G | (M + H)⁺ (ESI⁺), |
| | (thiomorpholin-4- | | | 2H), 2.67-2.62 (m, 8H), 2.09-2.00 (m, 1H), 1.08-0.87 (m, 4H). | | at 1.91 min, |
| | ylmethyl)benzamide | | | | | UV active |
| 1-2 | N-[4-(3-Cyclopropyl-1H-1,2,4- | A | B | ¹H NMR (400 MHz, DMSO-d₆) δ 13.88 (s, 0.3H), 13.62 (s, 0.7H), 10.45 (s, | System 1 | m/z 466 |
| | triazol-5-yl)phenyl]-3-[(2-methyl- | 4 and 6 | | 0.3H), 10.35 (s, 0.7H), 7.94-7.83 (m, 6H), 7.59-7.50 (m, 2H), 3.80- | Method A | (M + H)⁺ (ESI⁺), |
| | 1,1-dioxo-1,4-thiazinan-4- | | | 3.77 (m, 2H), 3.34-3.20 (m, 3H), 3.17-2.99 (m, 2H), 2.77-2.75 (m, 1H), | | at 1.75 min, |
| | yl)methyl]benzamide | | | 2.55-2.53 (m, 1H), 2.12-1.95 (m, 1H), 1.15 (d, J = 6.8 Hz, 3H), 1.06- | | UV active |
| | | | | 0.87 (m, 4H). One proton not observed. | | |
| 1-3 | N-[4-(3-Cyclopropyl-1H-1,2,4- | B | C | ¹H NMR (400 MHz, DMSO-d₆) δ 13.98-13.47 (m, 1H), 10.36 (br s, 1H), | System 1 | m/z 480 |
| | triazol-5-yl)phenyl]-3-[(2,2- | 9 | | 7.94-7.88 (m, 6H), 7.60-7.51 (m, 2H), 3.72 (s, 2H), 3.25-3.16 (m, 2H), | Method H | (M + H)⁺ (ESI⁺), |
| | dimethyl-1,1-dioxo-1,4-thiazinan- | | | 2.92-2.78 (m, 2H), 2.62 (s, 2H), 2.12-1.98 (br s, 1H), 1.29 (s, 6H), 1.09- | | at 1.93 min, |
| | 4-yl)methyl]benzamide | | | 0.82 (m, 4H). | | UV active |
| 2-1 | N-[4-(5-Cyclopropyl-1H-1,2,4- | — | — | Commercially available - Enamine, Product ID - Z4525934481 | System 5 | m/z 470 |
| | triazol-3-yl)phenyl]-5-[(1,1-dioxo- | | | | Method N | (M + H)⁺ (ESI⁺), |
| | 1,4-thiazinan-4-yl)methyl]-2- | | | | | at 2.69 min, |
| | fluorobenzamide | | | | | UV active |
| 2-2 | N-[4-(5-Cyclopropyl-1H-1,2,4- | — | — | Commercially available - Enamine, Product ID - Z4525934482 | System 5 | m/z 470 |
| | triazol-3-yl)phenyl]-3-[(1,1-dioxo- | | | | Method N | (M + H)⁺ (ESI⁺), |
| | 1,4-thiazinan-4-yl)methyl]-5- | | | | | at 2.88 min, |
| | fluorobenzamide | | | | | UV active |
| 2-3 | N-[4-(5-Cyclopropyl-1H-1,2,4- | — | — | Commercially available - Enamine, Product ID - Z4525934483 | System 5 | m/z 470 |
| | triazol-3-yl)phenyl]-3-[(1,1-dioxo- | | | | Method N | (M + H)⁺ (ESI⁺), |
| | 1,4-thiazinan-4-yl)methyl]-4- | | | | | at 2.68 min, |
| | fluorobenzamide | | | | | UV active |
| 2-4 | 3-Chloro-N-[4-(5-cyclopropyl-1H- | — | — | Commercially available - Enamine, Product ID - Z4525934486 | System 5 | m/z 486, 488 |
| | 1,2,4-triazol-3-yl)phenyl]-5-[(1,1- | | | | Method N | (M + H)⁺ (ESI⁺), |
| | dioxo-1,4-thiazinan-4- | | | | | at 2.79 min, |
| | yl)methyl]benzamide | | | | | UV active |
| 2-5 | 4-Chloro-N-[4-(5-cyclopropyl-1H- | — | — | Commercially available - Enamine, Product ID - Z4525934487 | System 5 | m/z 486, 488 |
| | 1,2,4-triazol-3-yl)phenyl]-3-[(1,1- | | | | Method N | (M + H)⁺ (ESI⁺), |
| | dioxo-1,4-thiazinan-4- | | | | | at 2.76 min, |
| | yl)methyl]benzamide | | | | | UV active |
| 3-1 | 3-[(1,1-Dioxo-1,4-thiazinan-4- | C | D | ¹H NMR (400 MHz, Methanol-d₄) δ 8.36 (s, 1H), 8.15-8.08 (m, 3H), 8.05 | System 2 | m/z 454 |
| | yl)methyl]-N-[4-(3-propan-2-yl-1H- | 11 and 12 | | (d, J = 8.9 Hz, 2H), 7.84 (d, J = 7.7 Hz, 1H), 7.69 (t, J = 7.7 Hz, 1H), 4.60 | Method I | (M + H)⁺ (ESI⁺), |
| | 1,2,4-triazol-5- | | | (s, 2H), 3.87-3.81 (m, 4H), 3.65 (br s, 4H), 3.43 (hept, J = 7.0 Hz, 1H), | | at 2.97 min, |
| | yl)phenyl]benzamide HCl salt | | | 1.53 (d, J = 7.1 Hz, 6H). Three exchangeable proton not observed. | | UV active |
| 3-2 | N-[4-(5-tert-Butyl-1H-1,2,4-triazol- | — | — | Commercially available - Enamine, Product ID - Z4511123495 | System 5 | m/z 468 |
| | 3-yl)phenyl]-3-[(1,1-dioxo-1,4- | | | | Method M | (M + H)⁺ (ESI⁺), |
| | thiazinan-4-yl)methyl]benzamide | | | | | at 1.18 min, |
| | | | | | | UV active |
| 3-3 | N-[4-(3-Cyclopropyl-1H-1,2,4- | — | — | Commercially available - Enamine, Product ID - Z1014255240 | System 5 | m/z 452 |
| | triazol-5-yl)phenyl]-3-[(1,1-dioxo- | | | | Method M | (M + H)⁺ (ESI⁺), |
| | 1,4-thiazinan-4- | | | | | at 1.03 min, |
| | yl)methyl]benzamide | | | | | UV active |
| 3-4 | N-[4-(3-Cyclobutyl-1H-1,2,4- | C | D | ¹H NMR (400 MHz, Deuterium Oxide) δ 7.88-7.81 (m, 2H), 7.79 (d, J = | System 3 | m/z 466 |
| | triazol-5-yl)phenyl]-3-[(1,1-dioxo- | 11 and 15 | | 8.8 Hz, 2H), 7.66-7.58 (m, 3H), 7.53 (t, J = 8.0 Hz, 1H), 4.38 (s, 2H), | Method J | (M + H)⁺ (ESI⁺), |
| | 1,4-thiazinan-4- | | | 3.77-3.64 (m, 5H), 3.56-3.42 (m, 4H), 2.42-2.29 (m, 2H), 2.29-2.17 | | at 1.82 min, |
| | yl)methyl]benzamide HCl salt | | | (m, 2H), 2.13-1.96 (m, 1H), 1.92-1.77 (m, 1H). Three exchangeable | | UV active |
| | | | | proton not observed. | | |
| 3-5 | N-[4-(3-Cyclopentyl-1H-1,2,4- | — | — | Commercially available - Enamine, Product ID - Z4445302411 | System 5 | m/z 480 |
| | triazol-5-yl)phenyl]-3-[(1,1-dioxo- | | | | Method M | (M + H)⁺ (ESI⁺), |
| | 1,4-thiazinan-4- | | | | | at 1.21 min, |
| | yl)methyl]benzamide | | | | | UV active |
| 3-6 | N-[4-(3-Cyclohexyl-1H-1,2,4- | — | — | Commercially available - Enamine, Product ID - Z4445302424 | System 5 | m/z 494 |
| | triazol-5-yl)phenyl]-3-[(1,1-dioxo- | | | | Method M | (M + H)⁺ (ESI⁺), |
| | 1,4-thiazinan-4- | | | | | at 1.27 min, |
| | yl)methyl]benzamide | | | | | UV active |
| 3-7 | N-[4-[3-(Cyclopropylmethyl)-1H- | — | — | Commercially available - Enamine, Product ID - Z4445302435 | System 5 | m/z 466 |
| | 1,2,4-triazol-5-yl]phenyl]-3-[(1,1- | | | | Method M | (M + H)⁺ (ESI⁺), |
| | dioxo-1,4-thiazinan-4- | | | | | at 1.14 min, |
| | yl)methyl]benzamide | | | | | UV active |
| 3-8 | 3-[(1,1-Dioxo-1,4-thiazinan-4- | — | — | Commercially available - Enamine, Product ID - Z4445302324 | System 5 | m/z 557 |
| | yl)methyl]-N-[4-[3-(1- | | | | Method N | (M + H)⁺ (ESI⁺), |
| | phenylpyrrolidin-3-yl)-1H-1,2,4- | | | | | at 2.87 min, |
| | triazol-5-yl]phenyl]benzamide | | | | | UV active |
| 3-9 | N-[4-[3-(1-Benzylpiperidin-3-yl)- | — | — | Commercially available - Enamine, Product ID - Z4445302379 | System 5 | m/z 585 |
| | 1H-1,2,4-triazol-5-yl]phenyl]-3- | | | | Method M | (M + H)⁺ (ESI⁺), |
| | [(1,1-dioxo-1,4-thiazinan-4- | | | | | at 0.99 min, |
| | yl)methyl]benzamide | | | | | UV active |
| 3-10 | 3-[(1,1-Dioxo-1,4-thiazinan-4- | — | — | Commercially available - Enamine, Product ID - Z4445302442 | System 5 | m/z 506 |
| | yl)methyl]-N-[4-[3-(3-fluorophenyl)- | | | | Method M | (M + H)⁺ (ESI⁺), |
| | 1H-1,2,4-triazol-5- | | | | | at 1.30 min, |
| | yl]phenyl]benzamide | | | | | UV active |
| 3-11 | N-[4-[3-(3-Chlorophenyl)-1H-1,2,4- | — | — | Commercially available - Enamine, Product ID - Z4445302447 | System 5 | m/z 522 |
| | triazol-5-yl]phenyl]-3-[(1,1-dioxo- | | | | Method M | (M + H)⁺ (ESI⁺), |
| | 1,4-thiazinan-4- | | | | | at 1.42 min, |
| | yl)methyl]benzamide | | | | | UV active |
| 3-12 | 3-[(1,1-Dioxo-1,4-thiazinan-4- | — | — | Commercially available - Enamine, Product ID - Z4445302454 | System 5 | m/z 518 |
| | yl)methyl]-N-[4-[5-(4- | | | | Method M | (M + H)⁺ (ESI⁺), |
| | methoxyphenyl)-1H-1,2,4-triazol- | | | | | at 1.26 min, |
| | 3-yl]phenyl]benzamide | | | | | UV active |
| 3-13 | 3-[(1,1-Dioxo-1,4-thiazinan-4- | — | — | Commercially available - Enamine, Product ID - Z4449486803 | System 5 | m/z 587 |
| | yl)methyl]-N-[4-[3-[3-(1,3-thiazol-2- | | | | Method M | (M + H)⁺ (ESI⁺), |
| | yloxy)phenyl]-1H-1,2,4-triazol-5- | | | | | at 1.33 min, |
| | yl]phenyl]benzamide | | | | | UV active |
| 3-14 | 3-[(1,1-Dioxo-1,4-thiazinan-4- | — | — | Commercially available - Enamine, Product ID - Z4449487304 | System 5 | m/z 612 |
| | yl)methyl]-N-[4-[3-[4-[(2- | | | | Method N | (M + H)⁺ (ESI⁺), |
| | fluorophenoxy)methyl]phenyl]-1H- | | | | | at 3.55 min, |
| | 1,2,4-triazol-5- | | | | | UV active |
| | yl]phenyl]benzamide | | | | | |
| 3-15 | 3-[(1,1-Dioxo-1,4-thiazinan-4- | — | — | Commercially available - Enamine, Product ID - Z4445302457 | System 5 | m/z 489 |
| | yl)methyl]-N-[4-(3-pyridin-2-yl-1H- | | | | Method M | (M + H)⁺ (ESI⁺), |
| | 1,2,4-triazol-5- | | | | | at 1.10 min, |
| | yl)phenyl]benzamide | | | | | UV active |
| 3-16 | 3-[(1,1-Dioxo-1,4-thiazinan-4- | — | — | Commercially available - Enamine, Product ID - Z4449495037 | System 5 | m/z 562 |
| | yl)methyl]-N-[4-[3-[5- | | | | Method M | (M + H)⁺ (ESI⁺), |
| | (trifluoromethyl)thiophen-2-yl]-1H- | | | | | at 1.36 min, |
| | 1,2,4-triazol-5- | | | | | UV active |
| | yl]phenyl]benzamide | | | | | |
| 3-17 | N-[4-(3-Benzyl-1H-1,2,4-triazol-5- | C | D | ¹H NMR (400 MHz, Methanol-d₄) δ 8.23 (s, 1H), 8.10-8.02 (m, 3H), 7.99 | System 3 | m/z 502 |
| | yl)phenyl]-3-[(1,1-dioxo-1,4- | 11 and 17 | | (d, J = 8.9 Hz, 2H), 7.77 (d, J = 7.6 Hz, 1H), 7.67 (t, J = 7.7 Hz, 1H), 7.43- | Method J | (M + H)⁺ (ESI⁺), |
| | thiazinan-4-yl)methyl]benzamide | | | 7.30 (m, 5H), 4.50 (s, 2H), 4.36 (s, 2H), 3.81-3.69 (m, 4H), 3.59-3.47 | | at 1.97 min, |
| | HCl salt | | | (m, 4H). Two exchangeable proton not observed. | | UV active |
| 3-18 | 3-[(1,1-Dioxo-1,4-thiazinan-4- | — | — | Commercially available - Enamine, Product ID - Z4445302481 | System 5 | m/z 516 |
| | yl)methyl]-N-[4-[5-[(4- | | | | Method M | (M + H)⁺ (ESI⁺), |
| | methylphenyl)methyl]-1H-1,2,4- | | | | | at 1.10 min, |
| | triazol-3-yl]phenyl]benzamide | | | | | UV active |
| 3-19 | 3-[(1,1-Dioxo-1,4-thiazinan-4- | — | — | Commercially available - Enamine, Product ID - Z4445302472 | System 5 | m/z 520 |
| | yl)methyl]-N-[4-[3-[(2- | | | | Method M | (M + H)⁺ (ESI⁺), |
| | fluorophenyl)methyl]-1H-1,2,4- | | | | | at 1.16 min, |
| | triazol-5-yl]phenyl]benzamide | | | | | UV active |
| 3-20 | 3-[(1,1-Dioxo-1,4-thiazinan-4- | — | — | Commercially available - Enamine, Product ID - Z4445302476 | System 5 | m/z 520 |
| | yl)methyl]-N-[4-[3-[(3- | | | | Method M | (M + H)⁺ (ESI⁺), |
| | fluorophenyl)methyl]-1H-1,2,4- | | | | | at 1.26 min, |
| | triazol-5-yl]phenyl]benzamide | | | | | UV active |
| 3-21 | 3-[(1,1-Dioxo-1,4-thiazinan-4- | — | — | Commercially available - Enamine, Product ID - Z4445302479 | System 5 | m/z 520 |
| | yl)methyl]-N-[4-[3-[(4- | | | | Method N | (M + H)⁺ (ESI⁺), |
| | fluorophenyl)methyl]-1H-1,2,4- | | | | | at 2.66 min, |
| | triazol-5-yl]phenyl]benzamide | | | | | UV active |
| 3-22 | 3-[(1, 1-Dioxo-1,4-thiazinan-4- | — | — | Commercially available - Enamine, Product ID - Z4445302487 | System 5 | m/z 532 |
| | yl)methyl]-N-[4-[5-[(3- | | | | Method M | (M + H)⁺ (ESI⁺), |
| | methoxyphenyl)methyl]-1H-1,2,4- | | | | | at 1.21 min, |
| | triazol-3-yl]phenyl]benzamide | | | | | UV active |
| 3-23 | N-[4-[3-[(4-Chlorophenyl)methyl]- | — | — | Commercially available - Enamine, Product ID - Z4445302484 | System 5 | m/z 536, 538 |
| | 1H-1,2,4-triazol-5-yl]phenyl]-3- | | | | Method M | (M + H)⁺ (ESI⁺), |
| | [(1,1-dioxo-1,4-thiazinan-4- | | | | | at 1.08 min, |
| | yl)methyl]benzamide | | | | | UV active |
| 3-24 | N-[4-[3-[(4-bromophenyl)methyl]- | — | — | Commercially available - Enamine, Product ID - Z4449485397 | System 5 | m/z 580, 582 |
| | 1H-1,2,4-triazol-5-yl]phenyl]-3- | | | | Method N | (M + H)⁺ (ESI⁺), |
| | [(1,1-dioxo-1,4-thiazinan-4- | | | | | at 1.25 min, |
| | yl)methyl]benzamide | | | | | UV active |
| 3-25 | N-[4-[5-[(3,4- | — | — | Commercially available - Enamine, Product ID - Z4449485739 | System 5 | m/z 530 |
| | Dimethylphenyl)methyl]-1H-1,2,4- | | | | Method N | (M + H)⁺ (ESI⁺), |
| | triazol-3-yl]phenyl]-3-[(1,1-dioxo- | | | | | at 2.91 min, |
| | 1,4-thiazinan-4- | | | | | UV active |
| | yl)methyl]benzamide | | | | | |
| 3-26 | N-[4-[3-[(2,5- | — | — | Commercially available - Enamine, Product ID - Z4449485768 | System 5 | m/z 570, 572, 574 |
| | Dichlorophenyl)methyl]-1H-1,2,4- | | | | Method N | (M + H)⁺ (ESI⁺), |
| | triazol-5-yl]phenyl]-3-[(1,1-dioxo- | | | | | at 1.05 |
| | 1,4-thiazinan-4- | | | | | min, UV active |
| | yl)methyl]benzamide | | | | | |
| 3-27 | N-[4-[5-[(4-Bromo-3- | — | — | Commercially available - Enamine, Product ID - Z4449495061 | System 5 | m/z 596, 598 |
| | methylphenyl)methyl]-1H-1,2,4- | | | | Method N | (M + H)⁺ (ESI⁺), |
| | triazol-3-yl]phenyl]-3-[(1,1-dioxo- | | | | | at 1.20 min, |
| | 1,4-thiazinan-4- | | | | | UV active |
| | yl)methyl]benzamide | | | | | |
| 3-28 | N-[4-[3-(2,3-Dihydro-1,4- | — | — | Commercially available - Enamine, Product ID - Z4449486415 | System 5 | m/z 560 |
| | benzodioxin-6-ylmethyl)-1H-1,2,4- | | | | Method M | (M + H)⁺ (ESI⁺), |
| | triazol-5-yl]phenyl]-3-[(1,1-dioxo- | | | | | at 1.12 min, |
| | 1,4-thiazinan-4- | | | | | UV active |
| | yl)methyl]benzamide | | | | | |
| 3-29 | N-[4-[3-[(6-Chloro-1,3- | — | — | Commercially available - Enamine, Product ID - Z4449486385 | System 5 | m/z 580, 582 |
| | benzodioxol-5-yl)methyl]-1H- | | | | Method M | (M + H)⁺ (ESI⁺), |
| | 1,2,4-triazol-5-yl]phenyl]-3-[(1,1- | | | | | at 1.18 min, |
| | dioxo-1,4-thiazinan-4- | | | | | UV active |
| | yl)methyl]benzamide | | | | | |
| 3-30 | 3-[(1,1-Dioxo-1,4-thiazinan-4- | — | — | Commercially available - Enamine, Product ID - Z4445302496 | System 5 | m/z 533 |
| | yl)methyl]-N-[4-[5-[(6- | | | | Method M | (M + H)⁺ (ESI⁺), |
| | methoxypyridin-3-yl)methyl]-1H- | | | | | at 1.12 min, |
| | 1,2,4-triazol-3- | | | | | UV active |
| | yl]phenyl]benzamide | | | | | |
| 3-31 | 3-[(1,1-Dioxo-1,4-thiazinan-4- | — | — | Commercially available - Enamine, Product ID - Z4445302499 | System 5 | m/z 516 |
| | yl)methyl]-N-[4-[3-(2-phenylethyl)- | | | | Method M | (M + H)⁺ (ESI⁺), |
| | 1H-1,2,4-triazol-5- | | | | | at 1.24 min, |
| | yl]phenyl]benzamide | | | | | UV active |
| 4-1 | N-[4-(4,5-Dimethyl-1,3-oxazol-2- | — | — | Commercially available - Enamine, Product ID - Z998985084 | System 5 | m/z 440 |
| | yl)phenyl]-3-[(1,1-dioxo-1,4- | | | | Method M | (M + H)⁺ (ESI⁺), |
| | thiazinan-4-yl)methyl]benzamide | | | | | at 1.28 min, |
| | | | | | | UV active |
| 4-2 | N-[4-(4-Cyclopropyl-5-methyl-1,3- | A | E | ¹H NMR (400 MHz, DMSO-d₆) δ 10.46 (br s, 1H), 7.93-7.85 (m, 6H), | System 1 | m/z 465 |
| | oxazol-2-yl)phenyl]-3-[(1,1-dioxo- | 11 and 21 | | 7.59-7.50 (m, 2H), 3.77 (s, 2H), 3.19-3.09 (m, 4H), 2.29-2.29 (m, 4H), | Method D | (M + H)⁺ (ESI⁺), |
| | 1,4-thiazinan-4- | | | 2.33 (s, 3H), 1.89-1.82 (m, 1H), 0.86-0.77 (m, 4H). | | at 2.32 min, |
| | yl)methyl]benzamide | | | | | UV active |
| 4-3 | N-[4-(4-Cyclopropyl-5-methyl-1,3- | C | E | ¹H NMR (400 MHz, DMSO-d₆) δ 10.52 (s, 1H), 8.11 (dd, J = 7.2, 2.3 Hz, | System 1 | m/z 484 |
| | oxazol-2-yl)phenyl]-3-[(1,1-dioxo- | 21 and 23 | | 1H), 8.07-8.01 (m, 1H), 7.99-7.88 (m, 4H), 7.45 (t, J = 9.1 Hz, 1H), | Method D | (M + H)⁺ (ESI⁺), |
| | 1,4-thiazinan-4-yl)methyl]-4- | | | 3.87 (s, 2H), 3.24-3.16 (m, 4H), 3.03-2.93 (m, 4H), 2.45 (s, 3H), 1.97- | | at 2.90 min, |
| | fluorobenzamide | | | 1.86 (m, 1H), 1.03-0.74 (m, 4H). | | UV active |
| 4-4 | 4-Chloro-N-[4-(4-cyclopropyl-5- | C | F | ¹H NMR (400 MHz, DMSO-d₆) δ 10.52 (br s, 1H), 8.08 (s, 1H), 7.92-7.86 | System 1 | m/z 500, 502 |
| | methyl-1,3-oxazol-2-yl)phenyl]-3- | 21 and 25 | | (m, 5H), 7.65 (d, J = 8.00 Hz, 1H), 3.84 (s, 2H), 3.20-3.14 (m, 4H), 3.10- | Method D | (M + H)⁺ (ESI⁺), |
| | [(1,1-dioxo-1,4-thiazinan-4- | | | 2.90 (m, 4H), 2.34 (s, 3H), 2.90-2.81 (m, 1H), 0.86-0.77 (m, 4H). | | at 2.69 min, |
| | yl)methyl]benzamide | | | | | UV active |
| 4-5 | N-[4-(2-Cyclopropyl-1,3-oxazol-5- | — | — | Commercially available - Enamine, Product ID - Z998514152 | System 5 | m/z 452 |
| | yl)phenyl]-3-[(1,1-dioxo-1,4- | | | | Method M | (M + H)⁺ (ESI⁺), |
| | thiazinan-4-yl)methyl]benzamide | | | | | at 1.27 min, |
| | | | | | | UV active |
| 4-6 | N-[4-(5-Cyclopropyl-1,2-oxazol-3- | A | F | ¹H NMR (400 MHz, DMSO-d₆) δ 10.48 (s, 1H), 8.01-7.84 (m, 4H), 7.80 | System 1 | m/z 452 |
| | yl)phenyl]-3-[(1,1-dioxo-1,4- | 11 and 27 | | (d, J = 8.9 Hz, 2H), 7.59 (d, J = 7.2 Hz, 1H), 7.53 (t, J = 7.8 Hz, 1H), 6.70 | Method C | (M + H)⁺ (ESI⁺), |
| | thiazinan-4-yl)methyl]benzamide | | | (s, 1H), 3.78 (s, 2H), 3.18-3.09 (m, 4H), 2.96-2.86 (m, 4H), 2.13-1.94 | | at 2.08 min, |
| | | | | (m, 1H), 1.13-0.99 (m, 2H), 0.88-0.71 (m, 2H). | | UV active |
| 4-7 | N-[4-(5-Cyclopropyl-1,2-oxazol-3- | A | G | ¹H NMR (400 MHz, DMSO-d₆) δ 10.44 (br s, 1H), 7.93-7.88 (m, 4H), | System 1 | m/z 452 |
| | yl)phenyl]-3-[(1,1-dioxo-1,4- | 11 and 29 | | 7.81 (d, J = 8.4 Hz, 2H), 7.59 (d, J = 7.6 Hz, 1H), 7.53 (t, J = 7.5 Hz, 1H), | Method A | (M + H)⁺ (ESI⁺), |
| | thiazinan-4-yl)methyl]benzamide | | | 6.72 (s, 1H), 3.78 (s, 2H), 3.18-3.09 (m, 4H), 2.95-2.83 (m, 4H), 2.21- | | at 2.07 min, |
| | | | | 2.15 (m, 1H), 1.13-1.08 (m, 2H), 0.96-0.92 (m, 2H). | | UV active |
| 4-8 | N-[4-(5-Cyclopropyl-1,3,4- | A | D | ¹H NMR (400 MHz, Acetonitrile-d₃) δ 12.67 (br s, 1H), 10.05 (s, 1H), 8.70 (s, | System 3 | m/z 453 |
| | oxadiazol-2-yl)phenyl]-3-[(1,1- | 11 and 32 | | 1H), 8.16 (d, J = 8.8 Hz, 2H), 8.04 (d, J = 7.7 Hz, 1H), 8.00 (d, J = 8.5 Hz, | Method J | (M + H)⁺ (ESI⁺), |
| | dioxo-1,4-thiazinan-4- | | | 2H), 7.68 (d, J = 7.6 Hz, 1H), 7.63 (t, J = 7.6 Hz, 1H), 4.47 (s, 2H), 4.29- | | at 1.97 min, |
| | yl)methyl]benzamide HCl salt | | | 4.05 (m, 2H), 3.92-3.55 (m, 4H), 3.39-3.17 (m, 2H), 2.26-2.09 (m, | | UV active |
| | | | | 1H), 1.23-1.11 (m, 4H). | | |
| 4-9 | N-[4-[5-(Dimethylamino)-1,3,4- | — | — | Commercially available - Enamine, Product ID - Z4445562697 | System 5 | m/z 456 |
| | oxadiazol-2-yl]phenyl]-3-[(1,1- | | | | Method M | (M + H)⁺ (ESI⁺), |
| | dioxo-1,4-thiazinan-4- | | | | | at 1.07 min, |
| | yl)methyl]benzamide | | | | | UV active |
| 4-10 | 3-[(1,1-Dioxo-1,4-thiazinan-4- | — | — | Commercially available - Enamine, Product ID - Z1090459400 | System 5 | m/z 455 |
| | yl)methyl]-N-[4-(3-propan-2-yl- | | | | Method M | (M + H)⁺ (ESI⁺), |
| | 1,2,4-oxadiazol-5- | | | | | at 1.39 min, |
| | yl)phenyl]benzamide | | | | | UV active |
| 4-11 | N-[4-(3-Cyclopropyl-1,2,4- | A | D | ¹H NMR (400 MHz, Methanol-d₄) δ 8.17 (s, 1H), 8.15-8.08 (m, 3H), 8.00 | System 3 | m/z 453 |
| | oxadiazol-5-yl)phenyl]-3-[(1,1- | 11 and 34 | | (d, J = 8.8 Hz, 2H), 7.78 (d, J = 7.7 Hz, 1H), 7.70 (t, J = 7.6 Hz, 1H), 4.49 | Method J | (M + H)⁺ (ESI⁺), |
| | dioxo-1,4-thiazinan-4- | | | (s, 2H), 3.79-3.67 (m, 4H), 3.56-3.44 (m, 4H), 2.23-2.11 (m, 1H), | | at 2.22 min, |
| | yl)methyl]benzamide HCl salt | | | 1.12 (ddt, J = 13.0, 5.2, 2.6 Hz, 4H). Two exchangeable proton not | | UV active |
| | | | | observed. | | |
| 4-12 | N-[4-(5-Cyclopropyl-1,2,4- | C | D | ¹H NMR (400 MHz, Methanol-d₄) δ 8.06 (s, 1H), 8.04-7.97 (m, 3H), 7.88 | System 3 | m/z 453 |
| | oxadiazol-3-yl)phenyl]-3-[(1,1- | 11 and 36 | | (d, J = 8.7 Hz, 2H), 7.70 (d, J = 7.6 Hz, 1H), 7.61 (t, J = 7.7 Hz, 1H), 4.23 | Method J | (M + H)⁺ (ESI⁺), |
| | dioxo-1,4-thiazinan-4- | | | (br s, 2H), 3.47 (br s, 4H), 3.35 (br s, 4H), 2.42-2.22 (m, 1H), 1.41-1.16 | | at 2.15 min, |
| | yl)methyl]benzamide HCl salt | | | (m, 4H). Two exchangeable proton not observed. | | UV active |
| 4-13 | N-[4-(5-Cyclobutyl-1,2,4- | C | H | ¹H NMR (400 MHz, Methanol-d₄) δ 8.08 (d, J = 8.8 Hz, 2H), 7.98 (s, 1H), | System 3 | m/z 467 |
| | oxadiazol-3-yl)phenyl]-3-[(1,1- | 11 and 38 | | 7.94-7.87 (m, 3H), 7.64 (d, J = 7.7 Hz, 1H), 7.54 (t, J = 7.6 Hz, 1H), 3.97- | Method J | (M + H)⁺ (ESI⁺), |
| | dioxo-1,4-thiazinan-4- | | | 3.84 (m, 1H), 3.81 (s, 2H), 3.21-3.12 (m, 4H), 3.07-2.99 (m, 4H), | | at 2.28 min, |
| | yl)methyl]benzamide | | | 2.64-2.48 (m, 4H), 2.34-2.18 (m, 1H), 2.18-2.04 (m, 1H). One | | UV active |
| | | | | exchangeable proton not observed. | | |
| 4-14 | N-[4-(5-Cyclopentyl-1,2,4- | C | I | ¹H NMR (400 MHz, | System 3 | m/z 481 |
| | oxadiazol-3-yl)phenyl]-3-[(1,1- | 11 and 40 | | Chloroform-d) δ 8.13 (d, J = 8.4 Hz, 2H), 7.96 (s, 1H), | Method J | (M + H)⁺ (ESI⁺), |
| | dioxo-1,4-thiazinan-4- | | | 7.90 (s, 1H), 7.86-7.76 (m, 3H), 7.61-7.48 (m, 2H), 3.76 (s, 2H), 3.53- | | at 2.39 min, |
| | yl)methyl]benzamide | | | 3.34 (m, 1H), 3.24-3.08 (m, 4H), 3.08-2.99 (m, 4H), 2.41-2.12 (m, | | UV active |
| | | | | 2H), 2.12-1.96 (m, 2H), 1.96-1.83 (m, 2H), 1.83-1.68 (m, 2H). | | |
| 4-15 | N-[4-(5-Benzyl-1,2,4-oxadiazol-3- | C | J | ¹H NMR (400 MHz, | System 3 | m/z 503 |
| | yl)phenyl]-3-[(1,1-dioxo-1,4- | 11 and 42 | | Methanol-d₄) δ 8.04 (d, J = 8.6 Hz, 2H), 7.95 (s, 1H), | Method J | (M + H)⁺ (ESI⁺), |
| | thiazinan-4-yl)methyl]benzamide | | | 7.92-7.84 (m, 3H), 7.61 (d, J = 7.7 Hz, 1H), 7.50 (t, J = 7.6 Hz, 1H), 7.42- | | at 2.35 min, |
| | | | | 7.27 (m, 5H), 4.35 (s, 2H), 3.78 (s, 2H), 3.17-3.07 (m, 4H), 3.06-2.94 | | UV active |
| | | | | (m, 4H). One exchangeable proton not observed. | | |
| 4-16 | 3-[(1,1-Dioxo-1,4-thiazinan-4- | C | K | ¹H NMR (400 MHz, Chloroform-d) δ 8.12 (d, J = 8.7 Hz, 2H), 7.92 (s, 1H), | System 3 | m/z 517 |
| | yl)methyl]-N-[4-[5-[(4- | 11 and 44 | | 7.90 (s, 1H), 7.84-7.74 (m, 3H), 7.59-7.48 (m, 2H), 7.32-7.27 (m, | Method J | (M + H)⁺ (ESI⁺), |
| | methylphenyl)methyl]-1,2,4- | | | 2H), 7.20 (d, J = 7.8 Hz, 2H), 4.28 (s, 2H), 3.76 (s, 2H), 3.14-3.08 (m, | | at 2.46 min, |
| | oxadiazol-3-yl]phenyl]benzamide | | | 4H), 3.07-3.00 (m, 4H), 2.37 (s, 3H). | | UV active |
| 4-17 | 3-[(1,1-Dioxo-1,4-thiazinan-4- | C | K | ¹H NMR (400 MHz, Chloroform-d) δ 8.12 (d, J = 8.7 Hz, 2H), 7.93 (s, 1H), | System 3 | m/z 521 |
| | yl)methyl]-N-[4-[5-[(4- | 11 and 46 | | 7.90 (s, 1H), 7.85-7.76 (m, 3H), 7.62-7.47 (m, 2H), 7.39 (dd, J = 8.6, | Method J | (M + H)⁺ (ESI⁺), |
| | fluorophenyl)methyl]-1,2,4- | | | 5.2 Hz, 2H), 7.08 (t, J = 8.6 Hz, 2H), 4.29 (s, 2H), 3.76 (s, 2H), 3.24-3.07 | | at 2.37 min, |
| | oxadiazol-3-yl]phenyl]benzamide | | | (m, 4H), 3.07-2.99 (m, 4H). | | UV active |
| 4-18 | N-[4-[5-[(4-Chlorophenyl)methyl]- | C | I | ¹H NMR (400 MHz, DMSO-d₆) δ 10.52 (s, 1H), 7.98 (s, 4H), 7.93-7.86 | System 3 | m/z 537 |
| | 1,2,4-oxadiazol-3-yl]phenyl]-3- | 11 and 48 | | (m, 2H), 7.59 (d, J = 7.5 Hz, 1H), 7.53 (t, J = 7.6 Hz, 1H), 7.46 (s, 4H), | Method J | (M + H)⁺ (ESI⁺), |
| | [(1,1-dioxo-1,4-thiazinan-4- | | | 4.46 (s, 2H), 3.77 (s, 2H), 3.13 (t, J = 5.2 Hz, 4H), 2.95-2.87 (m, 4H). | | at 2.48 min, |
| | yl)methyl]benzamide | | | | | UV active |
| 4-19 | N-[4-(5-Cyclopropyl-1H-pyrazol-3- | A | I | ¹H NMR (400 MHz, DMSO-d₆) δ 12.74 (s, 0.3H), 12.56 (s, 0.7H), 10.34 (s, | System 1 | m/z 451 |
| | yl)phenyl]-3-[(1,1-dioxo-1,4- | 11 and 50 | | 0.3H), 10.28 (s, 0.7H), 7.90-7.69 (m, 6H), 7.58-7.50 (m, 2H), 6.34 (s, | Method F | (M + H)⁺ (ESI⁺), |
| | thiazinan-4-yl)methyl]benzamide | | | 1H), 3.77 (s, 2H), 3.18-3.08 (m, 4H), 2.94-2.88 (m, 4H), 1.98-1.91 (m, | | at 1.51 min, |
| | | | | 1H), 0.97-0.87 (m, 2H), 0.73-0.69 (m, 2H). | | UV active |
| 4-20 | 4-Chloro-N-[4-(5-cyclopropyl-1H- | D | C | ¹H NMR (400 MHz, DMSO-d₆) δ 12.53 (br s, 1H), 10.38 (br s, 1H), 8.07 | System 1 | m/z 485 |
| | pyrazol-3-yl)phenyl]-3-[(1,1-dioxo- | 25 and 50 | | (d, J = 2.4 Hz, 1H), 7.91 (dd, J = 8.4, 2.4 Hz, 1H), 7.79 (d, J = 8.8 Hz, 2H), | Method A | (M + H)⁺ (ESI⁺), |
| | 1,4-thiazinan-4- | | | 7.72 (d, J = 8.8 Hz, 2H), 7.64 (d, J = 8.0 Hz, 1H), 6.34 (s, 1H), 3.84 (s, | | at 1.71 min, |
| | yl)methyl]benzamide | | | 2H), 3.17-3.14 (m, 4H), 3.00-2.99 (m, 4H), 1.94-1.90 (m, 1H), 0.95- | | UV active |
| | | | | 0.90 (m, 2H), 0.73-0.70 (m, 2H). | | |
| 4-21 | N-[4-(3-Benzyl-1H-pyrazol-5- | A | C | ¹H NMR (400 MHz, DMSO-d₆) δ 12.75 (br s, 1H), 10.30 (br s, 1H), 7.90- | System 1 | m/z 500 |
| | yl)phenyl]-3-[(1,1-dioxo-1,4- | 11 and 52 | | 7.86 (m, 2H), 7.80 (d, J = 8.8 Hz, 2H), 7.72 (d, J = 8.8 Hz, 2H), 7.60- | Method A | (M + H)⁺ (ESI⁺), |
| | thiazinan-4-yl)methyl]benzamide | | | 7.54 (m, 1H), 7.51 (t, J = 7.5 Hz, 1H), 7.32-7.28 (m, 4H), 7.24-7.20 (m, | | at 1.87 min, |
| | | | | 1H), 6.42 (s, 1H), 3.97 (s, 2H), 3.77 (s, 2H), 3.14-3.12 (m, 4H), 2.92- | | UV active |
| | | | | 2.91 (m, 4H). | | |
| 4-22 | 4-Chloro-N-[4-(5-cyclopropyl-4- | E | F | ¹H NMR (400 MHz, DMSO-d₆) δ 12.42 (s, 0.4H), 12.25 (s, 0.6H), 10.39 | System 4 | m/z 499, 501 |
| | methyl-1H-pyrazol-3-yl)phenyl]-3- | 25 and 53 | | (br s, 1H), 8.08 (d, J = 2.00 Hz, 1H), 7.92 (dd, J = 8.40, 2.0 Hz, 1H), 7.89- | Method K | (M + H)⁺ (ESI⁺), |
| | [(1,1-dioxo-1,4-thiazinan-4- | | | 7.80 (m, 2H), 7.66-7.60 (m, 3H), 3.85 (s, 2H), 3.16-3.15 (m, 4H), 3.02- | | at 2.65 min, |
| | yl)methyl]benzamide | | | 2.98 (m, 4H), 2.18 (s, 3H), 1.89-1.81 (m, 1H), 0.89-0.75 (m, 4H). | | UV active |
| 4-23 | N-[4-(5-Cyclopropyl-2- | A | C | ¹H NMR (400 MHz, DMSO-d₆) δ 10.27 (br s, 1H), 7.90-7.86 (m, 2H), | System 1 | m/z 465 |
| | methylpyrazol-3-yl)phenyl]-3-[(1,1- | 11 and 54 | | 7.78 (d, J = 8.7 Hz, 2H), 7.71 (d, J = 8.8 Hz, 2H), 7.60-7.54 (m, 1H), | Method C | (M + H)⁺ (ESI⁺), |
| | dioxo-1,4-thiazinan-4- | | | 7.51 (t, J = 7.5 Hz, 1H), 6.32 (s, 1H), 3.87 (s, 3H), 3.81 (s, 2H), 3.14-3.13 | | at 1.89 min, |
| | yl)methyl]benzamide | | | (m, 4H), 2.92-2.91 (m, 4H), 1.94-1.88 (m, 1H), 1.01-0.96 (m, 2H), 0.70- | | UV active |
| | | | | 0.67 (m, 2H). | | |
| 4-24 | 4-Chloro-N-[4-(5-cyclopropyl-1- | D | F | ¹H NMR (400 MHz, DMSO-d₆) δ 10.33 (br s, 1H), 8.07 (d, J = 2.00 Hz, | System 1 | m/z 499, 501 |
| | methylpyrazol-3-yl)phenyl]-3-[(1,1- | 25 and 54 | | 1H), 7.90 (dd, J = 8.20, 2.4 Hz, 1H), 7.77 (d, J = 8.80 Hz, 2H), 7.71 (d, J = | Method D | (M + H)⁺ (ESI⁺), |
| | dioxo-1,4-thiazinan-4- | | | 8.80 Hz, 2H), 7.63 (d, J = 8.00 Hz, 1H), 6.33 (s, 1H), 3.87 (s, 3H), 3.84 (s, | | at 2.76 min, |
| | yl)methyl]benzamide | | | 2H), 3.17-3.14 (m, 4H), 3.00-2.99 (m, 4H), 1.98-1.86 (m, 1H), 1.01- | | UV active |
| | | | | 0.96 (m, 2H), 0.70-0.66 (m, 2H). | | |
| 4-25 | N-[4-(5-Cyclopropyl-1,4- | A | E | ¹H NMR (400 MHz, Methanol-d₄) δ 8.02 (s, 1H), 7.97 (d, J = 7.60 Hz, 1H), | System 1 | m/z 479 |
| | dimethylpyrazol-3-yl)phenyl]-3- | 11 and 55 | | 7.82 (d, J = 8.8 Hz, 2H), 7.67 (d, J = 8.0 Hz, 1H), 7.60-7.57 (m, 3H), 4.06 | Method A | (M + H)⁺ (ESI⁺), |
| | [(1,1-dioxo-1,4-thiazinan-4- | | | (s, 2H), 3.96 (s, 3H), 3.33-3.28 (m, 8H), 2.21 (s, 3H), 1.79-1.75 (m, 1H), | | at 2.05 min, |
| | yl)methyl]benzamide | | | 1.14-1.09 (m, 2H), 0.80-0.76 (m, 2H). One exchangeable proton not | | UV active |
| | | | | observed. | | |
| 4-26 | 4-Chloro-N-[4-(5-cyclopropyl-1,4- | D | F | ¹H NMR (400 MHz, DMSO-d₆) δ 10.36 (br s, 1H), 8.07 (d, J = 2.3 Hz, 1H), | System 1 | m/z 513, 515 |
| | dimethylpyrazol-3-yl)phenyl]-3- | 25 and 55 | | 7.91 (dd, J = 8.40, 2.0 Hz, 1H), 7.80 (d, J = 8.80 Hz, 2H), 7.64 (d, J = 8.40 | Method D | (M + H)⁺ (ESI⁺), |
| | [(1,1-dioxo-1,4-thiazinan-4- | | | Hz, 1H), 7.58 (d, J = 8.80 Hz, 2H), 3.87-3.81 (m, 5H), 3.16-3.15 (m, | | at 2.84 min, |
| | yl)methyl]benzamide | | | 4H), 3.01-2.97 (m, 4H), 2.15 (s, 3H), 1.73-1.70 (m, 1H), 1.02-0.98 (m, | | UV active |
| | | | | 2H), 0.69-0.65 (m, 2H). | | |
| 4-27 | N-[4-(5-Cyclopropyl-1-methyl- | F | D | ¹H NMR (400 MHz, Methanol-d₄) δ 8.21 (s, 1H), 8.07 (d, J = 7.9 Hz, 1H), | System 3 | m/z 466 |
| | 1,2,4-triazol-3-yl)phenyl]-3-[(1,1- | Example 3-3 | | 7.98 (s, 4H), 7.77 (d, J = 7.7 Hz, 1H), 7.67 (t, J = 7.7 Hz, 1H), 4.51 (s, | Method J | (M + H)⁺ (ESI⁺), |
| | dioxo-1,4-thiazinan-4- | | | 2H), 3.80-3.71 (m, 4H), 3.57-3.49 (m, 4H), 2.39-2.28 (m, 1H), 1.45- | | at 1.87 min, |
| | yl)methyl]benzamide HCl salt | | | 1.34 (m, 2H), 1.33-1.24 (m, 2H). Two exchangeable proton not | | UV active |
| | | | | observed. | | |
| 5-1 | N-[6-(4-Bromo-5-methyl-1,3- | G | F | ¹H NMR (400 MHz, DMSO-d₆) δ 10.84 (br s, 1H), 8.87 (d, J = 1.60 Hz, | System 4 | m/z 539, 541 |
| | oxazol-2-yl)pyridin-3-yl]-4-chloro- | 25 and 58 | | 1H), 8.35 (dd, J = 8.80, 2.40 Hz, 1H), 8.15 (d, J = 2.0 Hz, 1H), 7.98 (dd, J | Method K | (M + H)⁺ (ESI⁺), |
| | 3-[(1,1-dioxo-1,4-thiazinan-4- | | | = 8.3, 2.2 Hz, 1H), 7.95 (d, J = 8.8 Hz, 1H), 7.56 (d, J = 8.40 Hz, 1H), 3.83 | | at 2.98 min, |
| | yl)methyl]benzamide | | | (s, 2H), 3.19-3.11 (m, 4H), 3.02-2.95 (m, 4H), 2.39 (s, 3H). | | UV active |
| 5-2 | 4-Chloro-N-[6-(4-cyclopropyl-5- | G | F | ¹H NMR (400 MHz, Methanol-d₄) δ 8.02 (s, 1H), 7.97 (d, J = 7.60 Hz, 1H), | System 4 | m/z 501, 503 |
| | methyl-1,3-oxazol-2-yl)pyridin-3- | 25 and 59 | | 7.82 (d, J = 8.8 Hz, 2H), 7.67 (d, J = 8.0 Hz, 1H), 7.60-7.57 (m, 3H), 4.06 | Method L | (M + H)⁺ (ESI⁺), |
| | yl]-3-[(1, 1-dioxo-1,4-thiazinan-4- | | | (s, 2H), 3.96 (s, 3H), 3.33-3.28 (m, 8H), 2.21 (s, 3H), 1.79-1.75 (m, 1H), | | at 3.24 min, |
| | yl)methyl]benzamide | | | 1.14-1.09 (m, 2H), 0.80-0.76 (m, 2H). One exchangeable proton not | | UV active |
| | | | | observed. | | |
| 5-3 | 4-Chloro-N-[6-(5-cyclopropyl-1H- | A | C | ¹H NMR (400 MHz, DMSO-d₆) δ 12.96 (s, 0.3H), 12.73 (s, 0.7H), 10.56 (s, | System 1 | m/z 486, 488 |
| | pyrazol-3-yl)pyridin-3-yl]-3-[(1,1- | 25 and 62 | | 1H), 8.91 (s, 1H), 8.20 (d, J = 7.20 Hz, 1H), 8.10 (d, J = 1.60 Hz, 1H), | Method D | (M + H)⁺ (ESI⁺), |
| | dioxo-1,4-thiazinan-4- | | | 8.05-8.02 (m, 2H), 7.66 (d, J = 8.40 Hz, 1H), 6.46 (s, 1H), 3.85 (s, 2H), | | at 2.28 min, |
| | yl)methyl]benzamide | | | 3.09 (s, 4H), 2.99 (s, 4H), 1.94-1.92 (m, 1H), 0.94-0.88 (m, 2H), 0.88- | | UV active |
| | | | | 0.73 (m, 2H). | | |
| 5-4 | N-[6-(5-Cyclopropyl-1- | A | F | ¹H NMR (400 MHz, DMSO-d₆) δ 10.48 (br s, 1H), 8.88 (s, 1H), 8.21 (dd, J | System 1 | m/z 466 |
| | methylpyrazol-3-yl)pyridin-3-yl]-3- | 11 and 63 | | = 8.6, 1.6 Hz, 1H), 7.93-7.86 (m, 3H), 7.60 (d, J = 7.6 Hz, 1H), 7.53 (t, J | Method A | (M + H)⁺ (ESI⁺), |
| | [(1,1-dioxo-1,4-thiazinan-4- | | | = 7.6 Hz, 1H), 6.42 (s, 1H), 3.90 (s, 3H), 3.78 (s, 2H), 3.21-3.12 (m, 4H), | | at 1.83 min, |
| | yl)methyl]benzamide | | | 2.98-2.89 (m, 4H), 1.94-1.90 (m, 1H), 1.00-0.98 (m, 2H), 0.71-0.70 | | UV active |
| | | | | (m, 2H). | | |
| 5-5 | N-[5-(5-Cyclopropyl-1- | H | C | ¹H NMR (400 MHz, DMSO-d₆) δ 10.84 (br s, 1H), 8.75 (s, 1H), 8.22 (d, J = | System 1 | m/z 466 |
| | methylpyrazol-3-yl)pyridin-2-yl]-3- | 65 and 66 | | 8.0 Hz, 1H), 8.14 (d, J = 7.2 Hz, 1H), 7.99 (s, 1H), 7.95 (d, J = 8.4 Hz, | Method H | (M + H)⁺ (ESI⁺), |
| | [(1,1-dioxo-1,4-thiazinan-4- | | | 1H), 7.57 (d, J = 7.2 Hz, 1H), 7.49 (t, J = 6.0 Hz, 1H), 6.46 (s, 1H), 3.90 | | at 2.03 min, |
| | yl)methyl]benzamide | | | (s, 3H), 3.76 (s, 2H), 3.18-3.08 (m, 4H), 2.96-2.86 (m, 4H), 1.98-1.88 | | UV active |
| | | | | (m, 1H), 1.05-0.95 (m, 2H), 0.72-0.68 (s, 2H). | | |

### Biological Activity

### Example A—Mouse Assay

Cell culture. All materials were purchased from Sigma-Aldrich, unless otherwise stated. Mouse embryonic fibroblasts (MEF) stably expressing mouse SMO^{WT}, SMO^{D473H} or SMO^{W535L} were maintained in Dulbecco's modified Eagle's medium (DMEM)/high-glucose medium supplemented with 10% foetal bovine serum (FBS, Invitrogen), 50 U/mL penicillin and 5 mg/mL streptomycin (supplied as a mixture), 0.1 mM MEM Non-Essential Amino Acids (supplied as 100× solution, ThermoFisher) and 1 mM sodium pyruvate at 37° C. in a humidified 95% air/5% CO₂ atmosphere.

Compound treatment. MEF mouse SMO^{WT}, SMO^{D473H} or SMO^{W535L} cells were seeded at 6000 cells per well in a Poly-D-Lysine coated 384-well plate (Greiner) before the spent culture medium was removed on the following day and replaced with assay medium (OptiMEM (Gibco) supplemented with 50 U/mL penicillin and 5 mg/mL streptomycin (supplied as a mixture), 0.1 mM MEM Non-Essential Amino Acids (supplied as 100× solution, ThermoFisher), 1 mM sodium pyruvate and 10 mM HEPES) containing test compounds at 2.5× final assay concentrations. After 1 hour incubation, cells were treated with assay medium containing mSHh prepared at 2.5× of final assay concentrations specified in the text and incubated for a further 24 hours at 37′C in a humidified 95% air/5% CO₂ atmosphere prior to qPCR analysis.

Cell lysis and one-step qPCR. The Cells-to-CT™ 1-Step TaqMan® Kit was used in conjunction with TaqMan primer-probe assays for the detection of Gli1 (FAM, Mm00494654_m1) and Gusb (VIC, Mm01197698_m1) genes (all purchased from Life Technologies). Following 24 hours cell treatment, spent medium was removed and cells were washed with PBS before the application of lysis and stop solution as per manufacturer's instructions. The qPCR reactions were run in a 384-well PCR plate using master mix prepared according to manufacturer's instructions and cell lysate samples from treatment wells. Real-time PCR amplification was detected using the Bio-Rad CFX384 Touch PCR system. Four experimental replicates (two cell replicates and two qPCR replicates) were performed for each compound concentration tested. Real-time data were analysed using the CFX Maestro software (Bio-Rad) and obtained expression values were converted to percentage Gli1 inhibition by normalising to the average Gli1 expression of mSHh-treated control samples.

TABLE 5
Data at mouse WT, D473H and W535L mutants for examples

| | Mouse | | | | | |
|---|---|---|---|---|---|---|
| | WT | | D473H | | W535L | |
| Example | pIC₅₀ | % Inh | pIC₅₀ | % Inh | pIC₅₀ | % Inh |
| Vismodegib | 8.4 | 98.0 | 5.8 | 78.8 | 6.8 | 78.3 |
| Example 1-1 | 7.7 | 99.4 | 6.7 | 99.3 | 6.6 | 66.8 |
| Example 1-2 | 7.8 | 97.3 | 6.9 | 95.6 | 6.7 | 63.3 |
| Example 1-3 | 7.7 | 99.4 | 7.2 | 99.7 | 6.8 | 62.6 |
| Example 2-1 | 7.8 | 96.5 | 6.7 | 95.3 | 7.1 | 68.2 |
| Example 2-2 | 8.0 | 95.8 | 6.9 | 91.3 | 6.9 | 67.2 |
| Example 2-3 | 7.7 | 94.4 | 6.8 | 93.9 | 7.1 | 76.5 |
| Example 2-4 | 8.3 | 95.4 | 7.4 | 95.4 | 7.4 | 60.2 |
| Example 2-5 | 7.8 | 95.5 | 6.8 | 94.4 | 7.0 | 72.2 |
| Example 3-1 | 7.7 | 99.1 | 6.8 | 96.6 | 6.7 | 77.1 |
| Example 3-2 | 7.6 | 97.3 | 6.3 | 91.0 | 7.0 | 41.6 |
| Example 3-3 | 7.8 | 96.6 | 6.8 | 89.9 | 6.8 | 61.9 |
| Example 3-4 | 7.6 | 98.0 | 6.7 | 97.7 | 6.7 | 63.0 |
| Example 3-5 | 8.0 | 96.6 | 7.3 | 96.7 | 7.8 | 42.2 |
| Example 3-6 | 7.5 | 98.4 | 6.7 | 98.4 | 7.1 | 51.7 |
| Example 3-7 | 7.6 | 98.5 | 6.7 | 97.3 | 6.7 | 70.7 |
| Example 3-8 | 7.7 | 98.6 | 6.8 | 98.0 | 7.0 | 57.9 |
| Example 3-9 | 7.5 | 95.1 | 6.5 | 93.8 | 7.2 | 63.2 |
| Example 3-10 | 7.9 | 99.1 | 6.6 | 97.9 | 7.0 | 63.9 |
| Example 3-11 | 8.0 | 98.4 | 7.0 | 98.5 | 6.3 | 62.5 |
| Example 3-12 | 7.9 | 99.0 | 6.7 | 97.5 | 6.5 | 53.3 |
| Example 3-13 | 7.1 | 97.4 | 6.2 | 78.5 | <5.48 | 76.7 |
| Example 3-14 | 7.4 | 97.3 | 6.6 | 95.2 | 6.2 | 50.9 |
| Example 3-15 | 7.1 | 98.9 | 6.0 | 97.0 | 6.4 | 53.4 |
| Example 3-16 | 7.0 | 99.4 | 6.1 | 93.9 | 6.1 | 58.5 |
| Example 3-17 | 8.2 | 99.4 | 7.0 | 99.0 | 7.4 | 54.3 |
| Example 3-18 | 8.5 | 98.5 | 7.1 | 98.1 | 7.6 | 65.7 |
| Example 3-19 | 7.6 | 99.1 | 6.5 | 96.9 | 7.4 | 64.3 |
| Example 3-20 | 7.8 | 99.0 | 6.6 | 96.8 | 7.6 | 66.1 |
| Example 3-21 | 7.0 | 97.5 | 6.1 | 82.3 | 6.3 | 64.1 |
| Example 3-22 | 8.1 | 98.5 | 7.2 | 98.2 | 7.5 | 60.1 |
| Example 3-23 | 8.1 | 99.5 | 6.5 | 95.0 | 7.6 | 58.2 |
| Example 3-24 | 8.4 | 98.8 | 7.3 | 98.5 | 7.4 | 72.5 |
| Example 3-25 | 8.7 | 98.5 | 7.0 | 97.4 | 7.4 | 80.5 |
| Example 3-26 | 7.8 | 98.3 | 6.3 | 87.1 | 6.6 | 64.8 |
| Example 3-27 | 7.8 | 99.3 | 6.2 | 97.0 | 6.8 | 57.3 |
| Example 3-28 | 7.1 | 99.4 | 6.1 | 97.7 | 6.5 | 59.7 |
| Example 3-29 | 7.6 | 99.1 | 6.6 | 98.9 | 6.9 | 64.8 |
| Example 3-30 | 7.1 | 99.0 | 6.2 | 98.4 | 6.3 | 69.1 |
| Example 3-31 | 7.5 | 98.6 | 6.6 | 97.2 | 7.1 | 64.1 |
| Example 4-1 | 8.2 | 99.2 | 7.3 | 98.6 | 7.2 | 78.6 |
| Example 4-2 | 9.2 | 97.5 | 8.6 | 98.4 | 7.8 | 75.3 |
| Example 4-3 | 9.7 | 97.1 | 8.7 | 97.4 | 8.0 | 71.9 |
| Example 4-4 | 9.7 | 99.5 | 8.5 | 98.5 | 7.9 | 79.0 |
| Example 4-5 | 8.1 | 99.9 | 7.0 | 99.7 | 6.8 | 63.2 |
| Example 4-6 | 7.9 | 99.0 | 7.1 | 99.3 | 6.9 | 76.9 |
| Example 4-7 | 8.6 | 98.8 | 7.7 | 99.3 | 7.5 | 76.0 |
| Example 4-8 | 7.1 | 98.9 | 6.1 | 94.9 | 6.3 | 65.9 |
| Example 4-9 | 7.4 | 99.6 | 6.5 | 99.1 | 6.1 | 70.0 |
| Example 4-10 | 8.2 | 97.8 | 7.9 | 98.1 | 7.2 | 54.8 |
| Example 4-11 | 7.3 | 99.7 | 6.8 | 99.5 | 6.8 | 63.8 |
| Example 4-12 | 8.0 | 99.6 | 7.4 | 99.8 | 6.8 | 57.8 |
| Example 4-13 | 7.3 | 98.8 | 6.5 | 99.0 | 6.3 | 77.4 |
| Example 4-14 | 8.0 | 98.2 | 6.7 | 99.1 | 6.1 | 74.2 |
| Example 4-15 | 8.2 | 99.0 | 7.5 | 99.4 | 7.5 | 82.0 |
| Example 4-16 | 8.7 | 97.5 | 7.9 | 98.8 | 7.7 | 72.5 |
| Example 4-17 | 8.7 | 97.2 | 8.1 | 97.4 | 8.2 | 68.6 |
| Example 4-18 | 8.6 | 98.7 | 7.8 | 99.2 | 7.4 | 82.7 |
| Example 4-19 | 8.3 | 96.8 | 7.8 | 97.7 | 8.0 | 59.5 |
| Example 4-20 | 8.6 | 99.1 | 7.5 | 98.4 | 8.0 | 51.6 |
| Example 4-21 | 8.2 | 99.3 | 7.2 | 99.4 | 7.4 | 76.6 |
| Example 4-22 | 8.7 | 97.0 | 8.3 | 97.6 | 8.4 | 49.2 |
| Example 4-23 | 8.7 | 99.1 | 7.6 | 99.0 | 7.6 | 77.7 |
| Example 4-24 | 9.2 | 97.9 | 8.1 | 98.1 | 8.0 | 70.0 |
| Example 4-25 | 9.4 | 98.3 | 8.3 | 98.6 | 7.8 | 65.7 |
| Example 4-26 | 9.1 | 97.0 | 8.2 | 97.6 | 8.3 | 58.6 |
| Example 4-27 | 8.2 | 99.5 | 7.3 | 98.5 | 6.8 | 77.7 |
| Example 5-1 | 7.7 | 99.3 | N.D | N.D | N.D | N.D |
| Example 5-2 | 8.7 | 98.8 | 8.1 | 98.1 | 7.2 | 82.7 |
| Example 5-3 | 7.2 | 98.6 | N.D | N.D | N.D | N.D |
| Example 5-4 | 8.1 | 97.3 | 6.9 | 97.0 | 6.6 | 66.4 |
| Example 5-5 | 7.5 | 97.7 | 7.0 | 93.2 | 6.2 | 61.1 |

N.D. = not determined

### Example B—Human Assay

Cell culture. All materials were purchased from Sigma-Aldrich, unless otherwise stated. SMO knock-out mouse embryonic fibroblasts (MEF^{SMO-/-}) were maintained in Dulbecco's modified Eagle's medium (DMEM)/high-glucose medium supplemented with 10% foetal bovine serum (FBS, Invitrogen), 50 U/mL penicillin and 5 mg/mL streptomycin (supplied as a mixture), 0.1 mMMEM Non-Essential Amino Acids (supplied as 100× solution, ThermoFisher) and 1 mM sodium pyruvate at 37° C. in a humidified 95% air/5% CO₂ atmosphere.

BacMam transduction and compound treatment. MEF^{SMO-/-} cells were transduced with BacMam virus (MOI 1:10 in the presence of 4.2 mM valproic acid) containing either the hSMO WT or desired receptor variant sequence. Transduced cells were seeded at 6000 cells per well in a Poly-D-Lysine coated 384-well plate (Greiner) before the spent culture medium was removed on the following day and replaced with assay medium (OptiMEM (Gibco) supplemented with 50 U/mL penicillin and 5 mg/mL streptomycin (supplied as a mixture), 0.1 mMMEM Non-Essential Amino Acids (supplied as 100× solution, ThermoFisher), 1 mM sodium pyruvate and 10 mM HEPES) containing test compounds at 2.5× final assay concentrations. After 1 hour incubation, cells were treated with assay medium containing mSHh prepared at 2.5× of final assay concentrations specified in the text and incubated for a further 24 hours at 37° C. in a humidified 95% air/5% ° C.2 atmosphere prior to qPCR analysis.

Cell lysis and one-step qPCR. The Cells-to-CT™ 1-Step TaqMan® Kit was used in conjunction with TaqMan primer-probe assays for the detection of Gill (FAM, Mm00494654_m1) and Gusb (VIC, Mm01197698_m1) genes (all purchased from Life Technologies). Following 24 hours cell treatment, spent medium was removed and cells were washed with PBS before the application of lysis and stop solution as per manufacturer's instructions. The qPCR reactions were run in a 384-well PCR plate using master mix prepared according to manufacturer's instructions and cell lysate samples from treatment wells. Real-time PCR amplification was detected using the Bio-Rad CFX384 Touch PCR system. Four experimental replicates (two cell replicates and two qPCR replicates) were performed for each compound concentration tested. Real-time data were analysed using the CFX Maestro software (Bio-Rad) and obtained expression values were converted to percentage Gill inhibition by normalising to the average Gill expression of mSHh-treated control samples

TABLE 6
Data at human WT, D473H and W535L
mutants for selected examples

| | Human | | | | | |
|---|---|---|---|---|---|---|
| | WT | | D473H | | W535L | |
| Example | pIC₅₀ | % Inh | pIC₅₀ | % Inh | pIC₅₀ | % Inh |
| Vismodegib | 8.3 | 100.1 | 5.1 | 45.4 | 6.7 | 92.6 |
| Example 1-2 | N.D. | N.D. | 6.7 | 85 | 7.7 | 78.2 |
| Example 2-2 | N.D. | N.D. | 6.1 | 73.6 | 8.1 | 79.1 |
| Example 2-4 | N.D. | N.D. | 6.2 | 76.5 | 8.3 | 87.8 |
| Example 3-3 | 7.5 | 85.9 | 5.9 | 77.2 | 7 | 88.4 |
| Example 3-5 | 8.7 | 83.4 | 6.6 | 88.3 | 8.6 | 75.4 |
| Example 3-24 | 8.2 | 94 | 7.6 | 104 | 7.6 | 98.5 |
| Example 4-2 | 9.3 | 97.8 | 7.5 | 104.2 | 7.6 | 104.6 |
| Example 4-3 | 9.5 | 93.6 | 7.6 | 103.2 | 7.9 | 95.6 |
| Example 4-10 | 8.3 | 88.4 | 7.3 | 100.2 | 7.9 | 77.8 |
| Example 4-17 | 9.1 | 99.3 | 7.4 | 114.2 | 7.7 | 102.7 |
| Example 4-19 | 8.5 | 95.4 | 7 | 107.1 | 7.9 | 86.3 |
| Example 4-22 | 8.9 | 98.6 | 7 | 111.1 | 7.8 | 89.9 |
| Example 4-23 | 9.4 | 123.1 | N.D. | N.D. | N.D. | N.D. |
| Example 4-26 | 9.1 | 99.2 | 7.2 | 111.9 | 8.2 | 92.5 |
| Example 5-4 | N.D. | N.D. | 6.1 | 107.7 | 7.4 | 85.9 |

N.D. = not determined

1. A compound of Formula (1a): 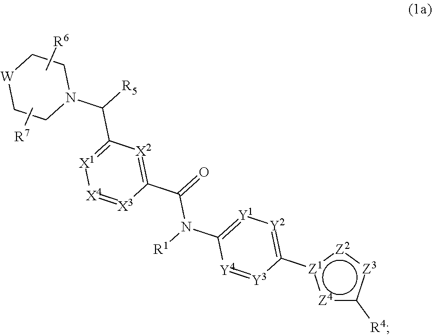
or a salt thereof, wherein;
W is S, SO or SO₂;
X¹, X², X³ and X⁴ are N, CH or CR², where one or none of X¹, X², X³ and X⁴ are N and one or none of X¹, X², X³ and X⁴ are CR²;
Y¹, Y², Y³ and Y⁴ are N, CH or CR³, where one or none of Y¹, Y², Y³ and Y⁴ are N and one or none of Y¹, Y², Y³ and Y⁴ are CR³;
Z¹ is C or N;
Z² is O, N, NR⁸, CR⁸ or S;
Z³ is O, N, NR⁹, CR⁹ or S;
Z⁴ is O, N, NR¹⁰, CR¹⁰ or S;
R¹ is H or methyl;
R² is H, CN, halo, C₁₋₃ alkyl optionally substituted with 1-3 fluorine atoms or C₁₋₃ alkoxy optionally substituted with 1-3 fluorine atoms;
R³ is H, halo or methyl;
R⁴ is H, halo, NR¹¹R¹², a group —(CH₂)ₙR¹³, where n is 0-3, or linear or branched C₁₋₆ alkyl optionally substituted with 1-3 fluorine atoms;
R⁵, R⁶ and R⁷ are independently H or C₁₋₃ alkyl optionally substituted with 1-3 fluorine atoms;
R⁸, R⁹ and R¹⁰ are independently H or C₁₋₃ alkyl optionally substituted with 1-3 fluorine atoms;
R¹¹ and R¹² are independently H or C₁₋₃ alkyl;
R¹³ is optionally substituted C₃₋₆ cycloalkyl, optionally substituted phenyl or an optionally substituted 5- or 6-membered heterocyclic ring;
wherein, when 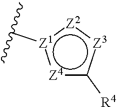
is: 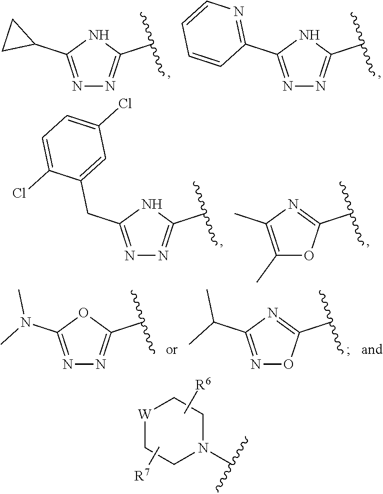
is 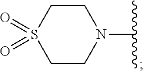 [SMILES: *N1CCS(=O)(=O)CC1]
X¹, X², X³, X⁴, Y¹, Y², Y³ and Y⁴ are not all CH, or R¹ and R⁵ are not both H.

2. The compound according to claim 1, which is a compound of formula (2a): 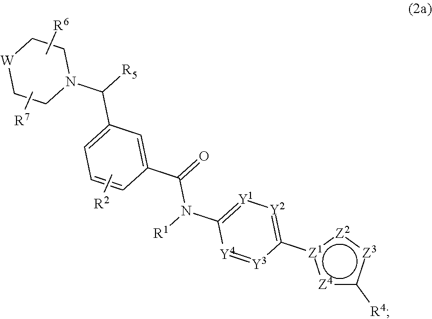
or a salt thereof.

3. The compound according to claim 1, which is a compound of formula (3a), (3b) or (3c): 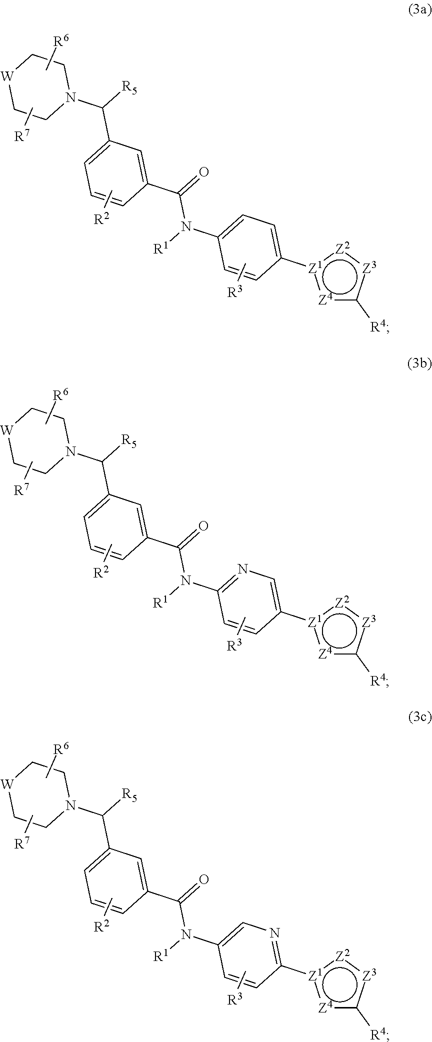
or a salt thereof.

4. The compound according to claim 1, wherein the moiety: 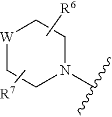
is selected from:

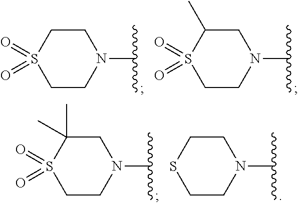

5. The compound according to claim 1, which is a compound of Formula (4a), (4b) or (4c): 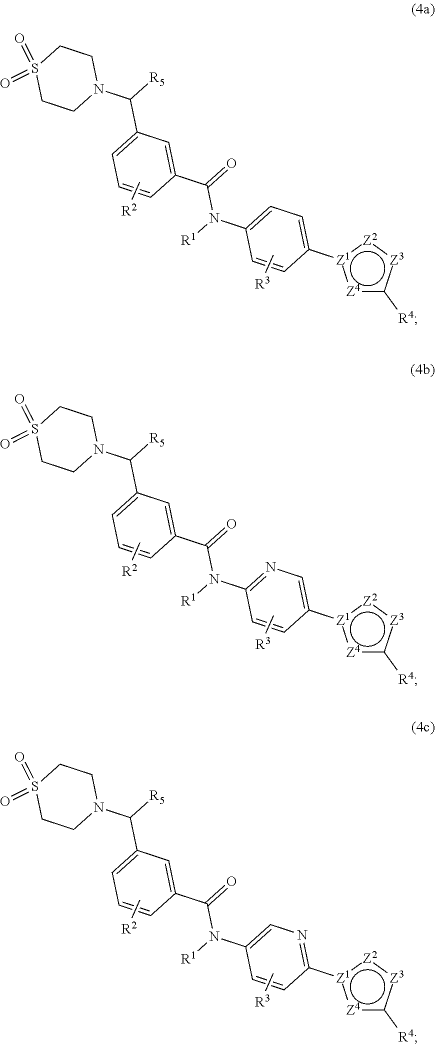
or a salt thereof.

6. The compound according to claim 1, wherein the moiety: 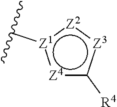
is selected from:

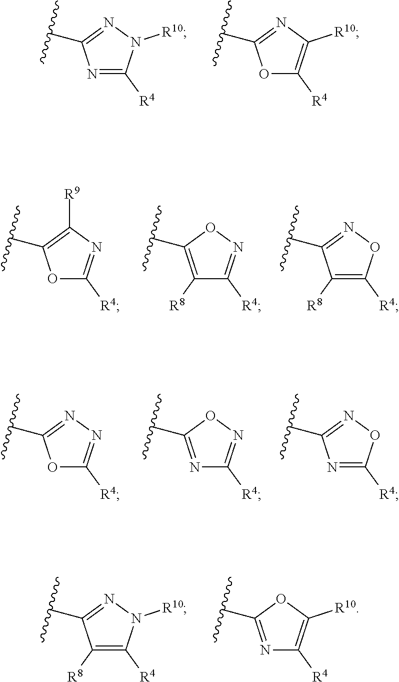

7. The compound according to claim 1, wherein R⁸, R⁹ and R¹⁰ are independently H or methyl.

8. The compound according to claim 1, wherein R⁴ is H, halo, NR¹¹R¹², a group —(CH₂)ₙR¹³, where n is 0-3, or linear or branched C₁₋₆ alkyl optionally substituted with 1-3 fluorine atoms;
R¹³ is C₃₋₆ cycloalkyl, phenyl optionally substituted with one or more R¹⁴ groups, a 5- or 6-membered heterocyclic group optionally substituted with one or more R¹⁴ groups, a 1,4-benzodioxane ring system optionally substituted with one or more R¹⁴ groups, or a 1,3 benzodioxole ring system optionally substituted with one or more R¹⁴ groups;
wherein R¹⁴ is halo, C₁₋₃ alkyl optionally substituted with 1-3 fluorine atoms, C₁₋₃ alkoxy optionally substituted with 1-3 fluorine atoms, or a group —(CH₂)ₘ(O)ₚR¹⁵;
where R¹⁵ is phenyl optionally substituted with 1-3 fluorine atoms or thiazole;
m is 0-3; and
p is 0 or 1.

9. The compound according to claim 1, wherein R⁴ is selected from:

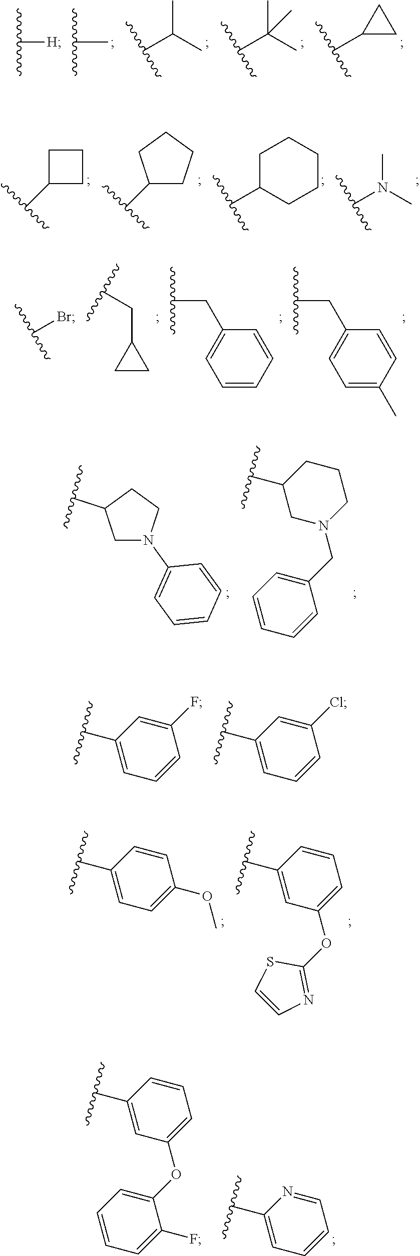

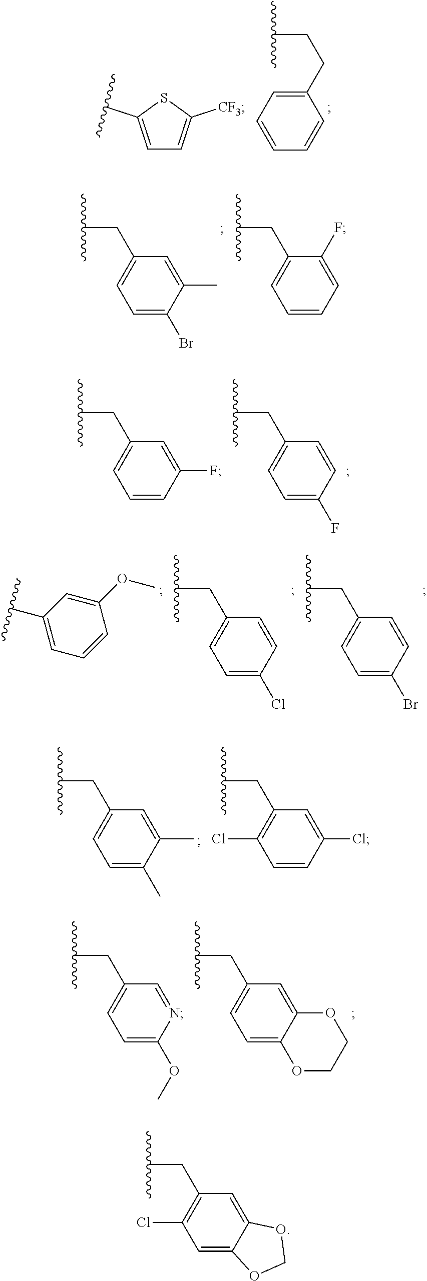

10. (canceled)

11. The compound according to claim 1, wherein the moiety: 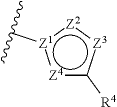
is selected from:

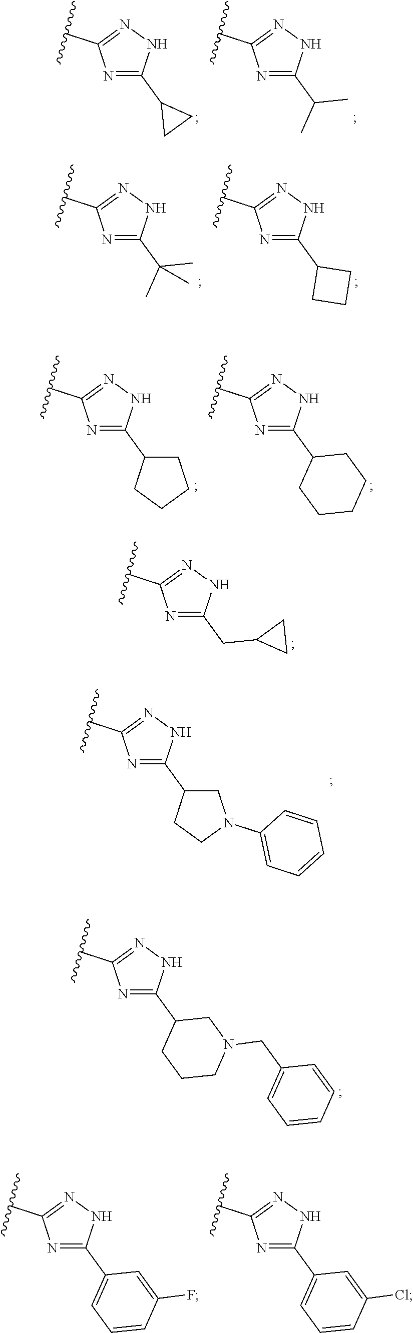

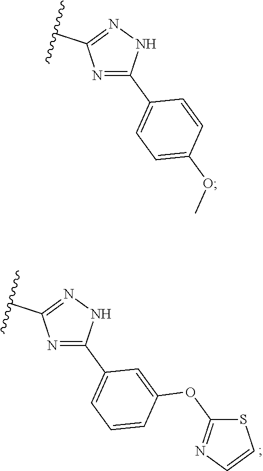 [SMILES: *c1n[nH]c(-c2ccc(OC)cc2)n1 ; *c1n[nH]c(-c2cccc(Oc3nccs3)c2)n1]

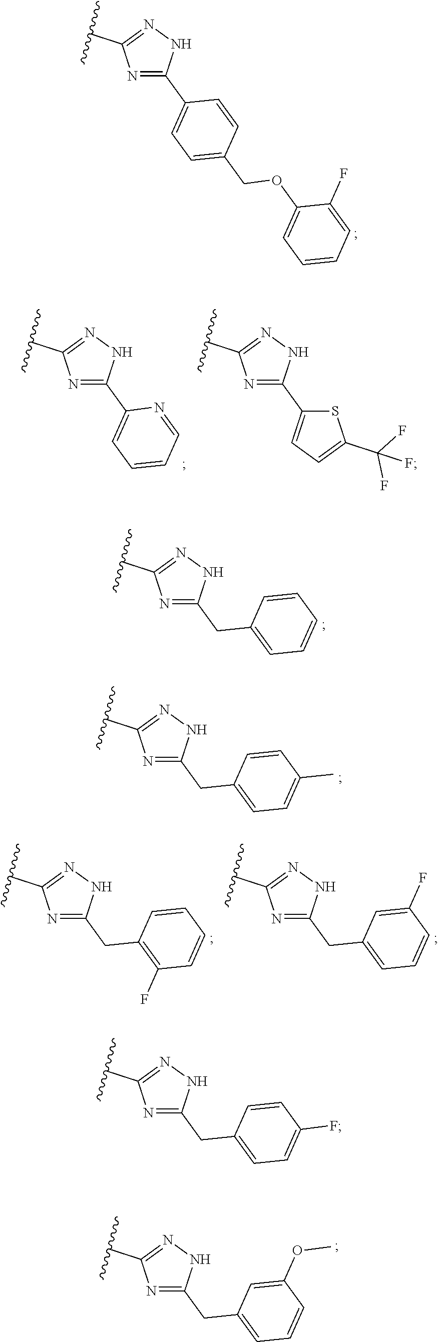

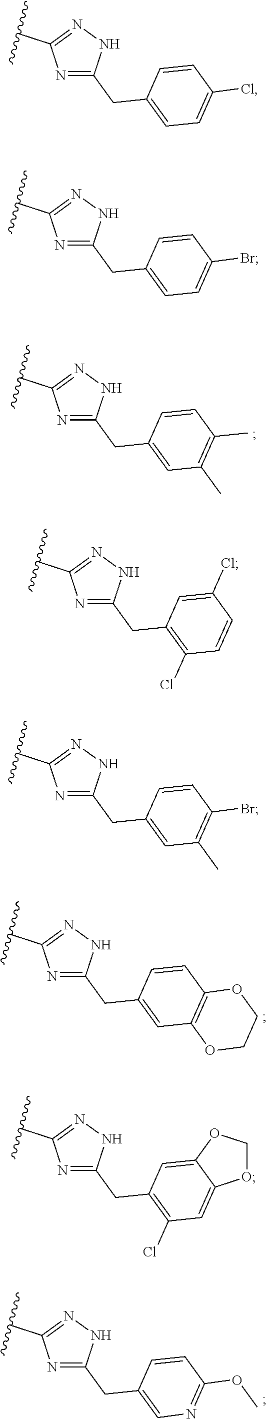

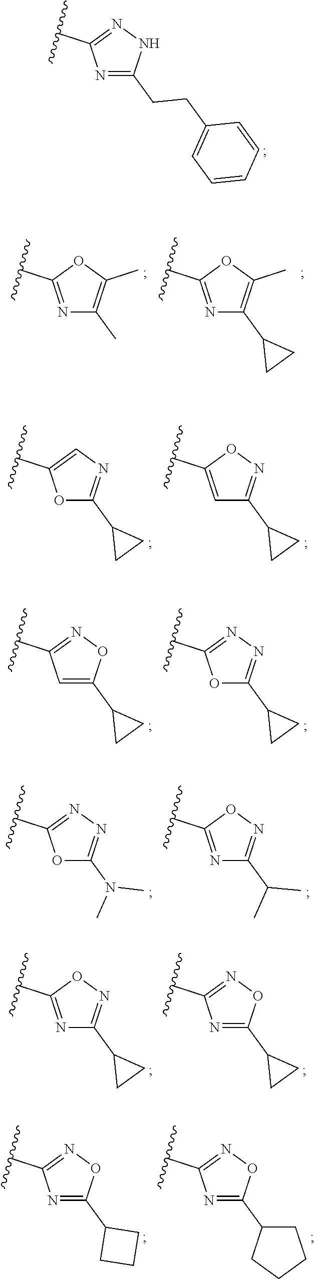

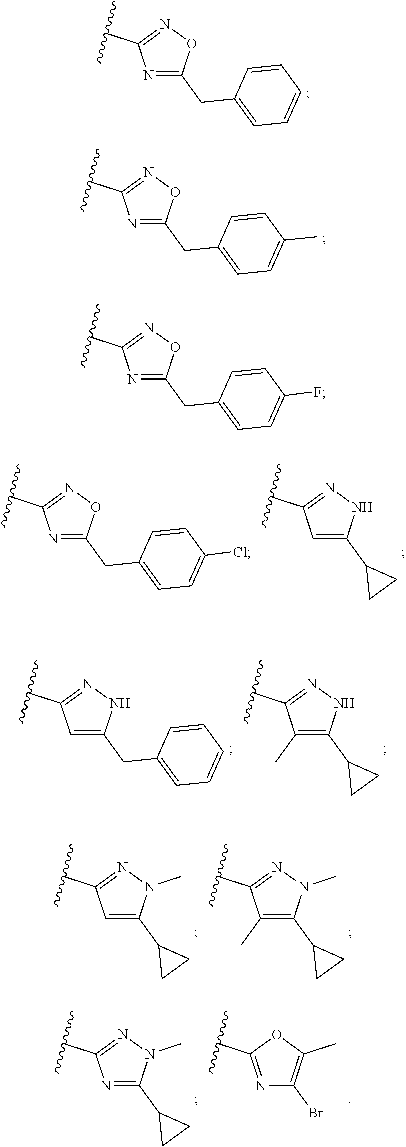

12. (canceled)

13. The compound according to claim 1, which is a compound of Formula (5a), (5b), (5c), (5d) or (5e): 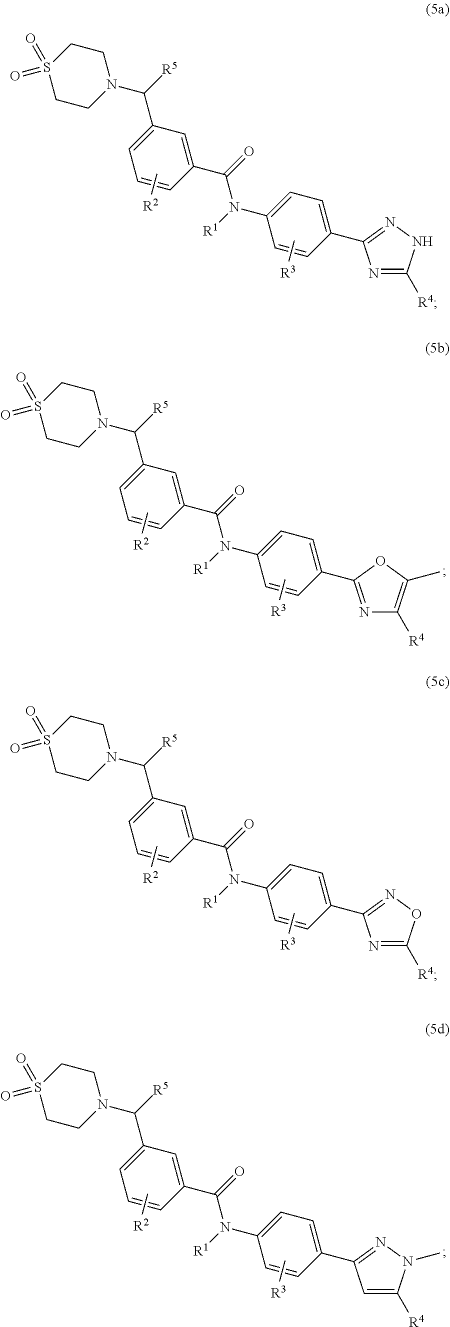 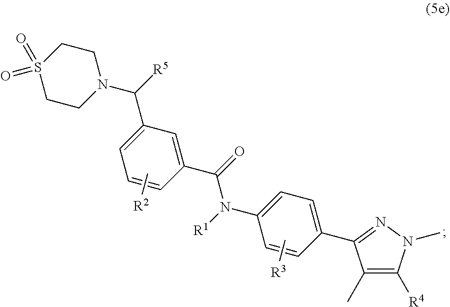
or a salt thereof.

14. The compound according to claim 1, wherein R¹ is H.

15. The compound according to claim 1, wherein R² is H, methyl, Cl, F, methoxy, trifluoromethyl or CN.

16. (canceled)

17. The compound according to claim 1, wherein R³ is H, methyl or F.

18. (canceled)

19. The compound according to claim 1, wherein R⁵ is H or methyl.

20. (canceled)

21. The compound according to claim 1, which is selected from the group consisting of:

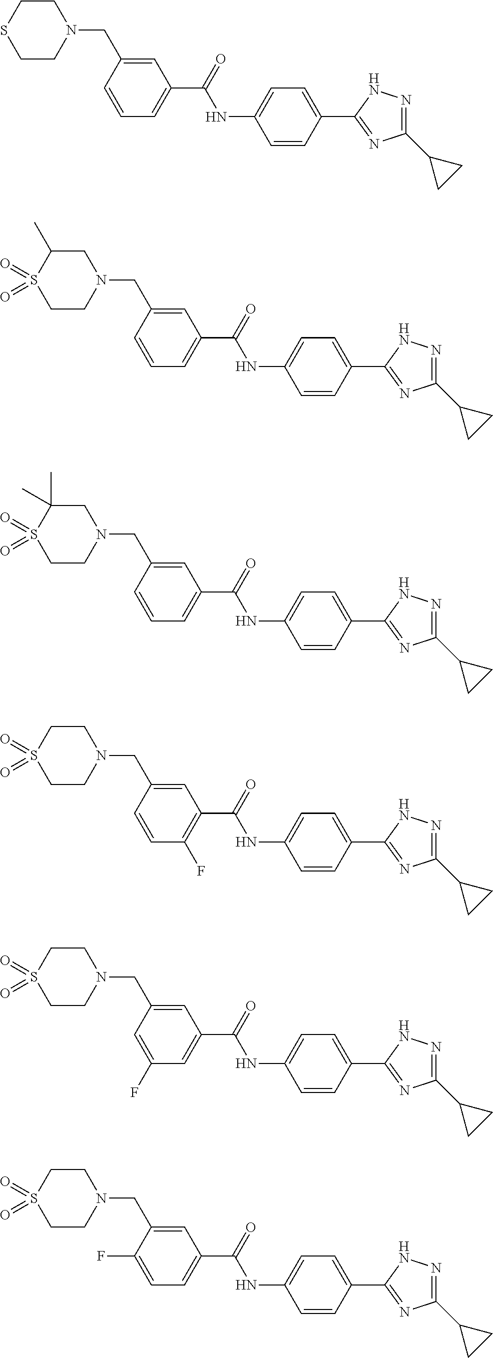 [SMILES: O=C(Nc1ccc(-c2nc(C3CC3)n[nH]2)cc1)c1cccc(CN2CCSCC2)c1 ; CC1CN(Cc2cccc(C(=O)Nc3ccc(-c4nc(C5CC5)n[nH]4)cc3)c2)CCS1(=O)=O ; CC1(C)CN(Cc2cccc(C(=O)Nc3ccc(-c4nc(C5CC5)n[nH]4)cc3)c2)CCS1(=O)=O ; O=C(Nc1ccc(-c2nc(C3CC3)n[nH]2)cc1)c1cc(CN2CCS(=O)(=O)CC2)ccc1F ; O=C(Nc1ccc(-c2nc(C3CC3)n[nH]2)cc1)c1cc(F)cc(CN2CCS(=O)(=O)CC2)c1 ; O=C(Nc1ccc(-c2nc(C3CC3)n[nH]2)cc1)c1ccc(F)c(CN2CCS(=O)(=O)CC2)c1]

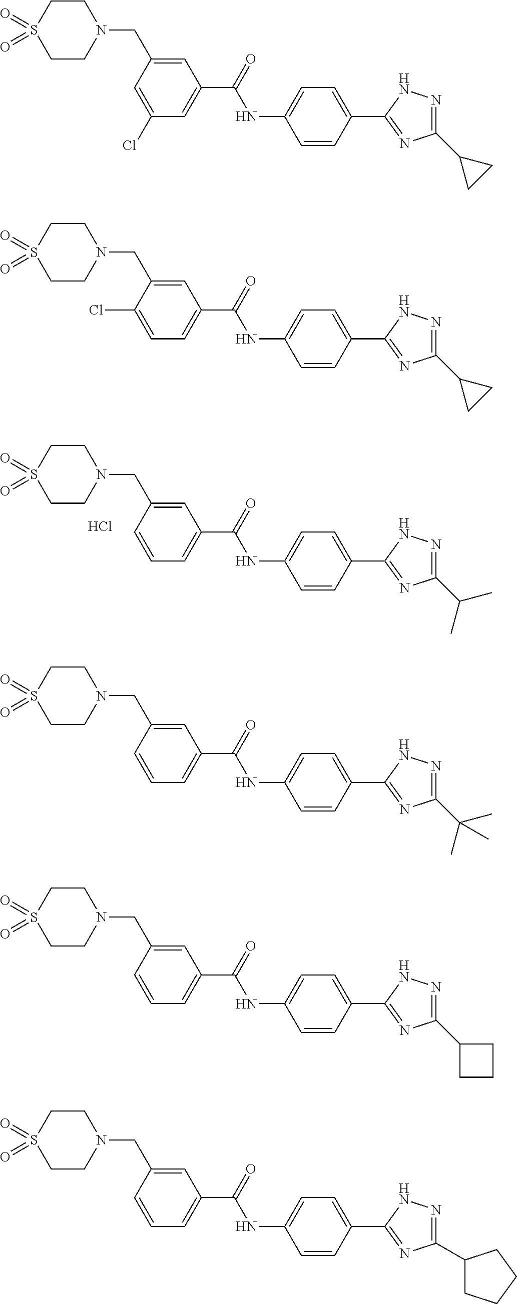

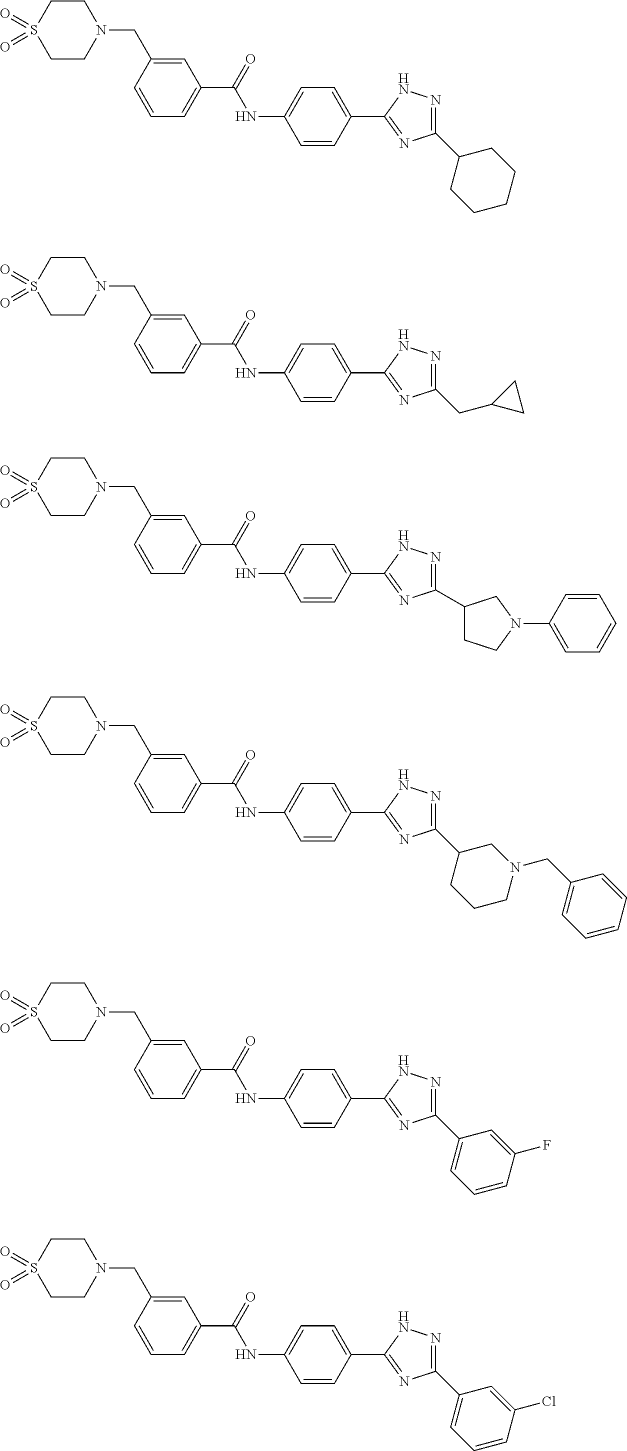 [SMILES: O=C(Nc1ccc(-c2nc(C3CCCCC3)n[nH]2)cc1)c1cccc(CN2CCS(=O)(=O)CC2)c1 ; O=C(Nc1ccc(-c2nc(CC3CC3)n[nH]2)cc1)c1cccc(CN2CCS(=O)(=O)CC2)c1 ; O=C(Nc1ccc(-c2nc(C3CCN(c4ccccc4)C3)n[nH]2)cc1)c1cccc(CN2CCS(=O)(=O)CC2)c1 ; O=C(Nc1ccc(-c2nc(C3CCCN(Cc4ccccc4)C3)n[nH]2)cc1)c1cccc(CN2CCS(=O)(=O)CC2)c1 ; O=C(Nc1ccc(-c2nc(-c3cccc(F)c3)n[nH]2)cc1)c1cccc(CN2CCS(=O)(=O)CC2)c1 ; O=C(Nc1ccc(-c2nc(-c3cccc(Cl)c3)n[nH]2)cc1)c1cccc(CN2CCS(=O)(=O)CC2)c1]

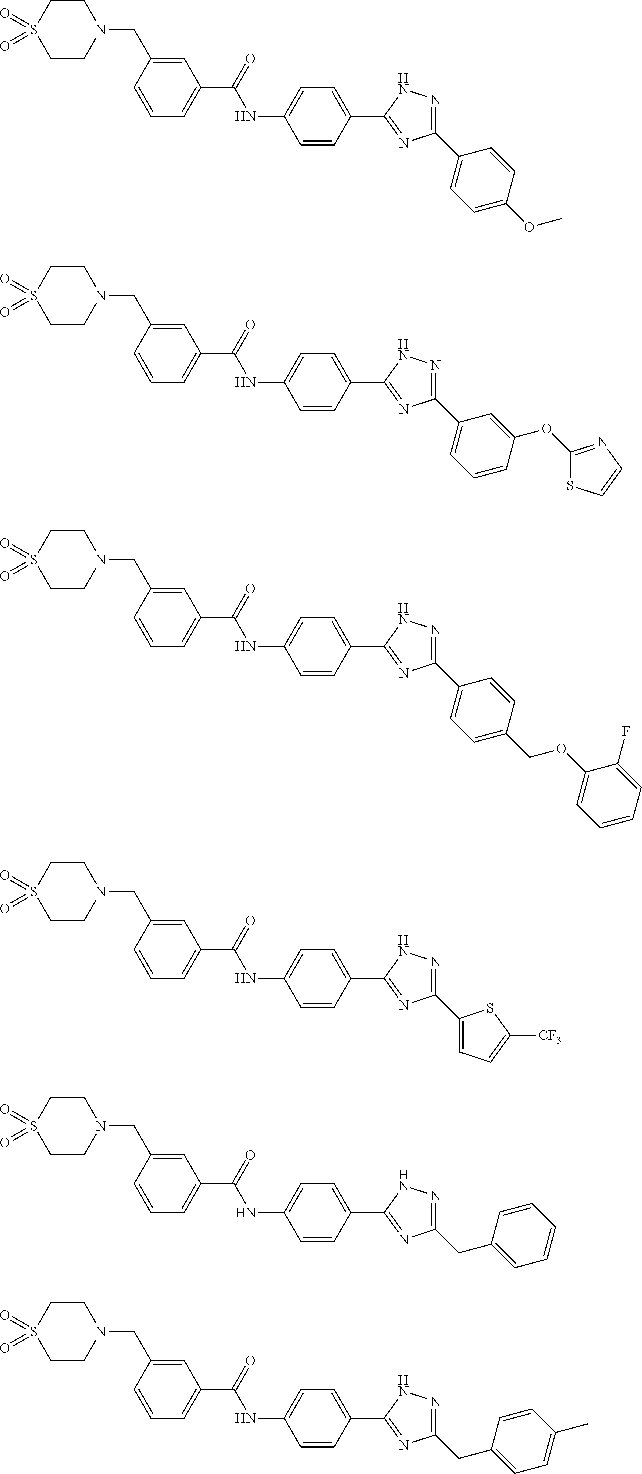 [SMILES: COc1ccc(-c2n[nH]c(-c3ccc(NC(=O)c4cccc(CN5CCS(=O)(=O)CC5)c4)cc3)n2)cc1 ; O=C(Nc1ccc(-c2nc(-c3cccc(Oc4nccs4)c3)n[nH]2)cc1)c1cccc(CN2CCS(=O)(=O)CC2)c1 ; O=C(Nc1ccc(-c2nc(-c3ccc(COc4ccccc4F)cc3)n[nH]2)cc1)c1cccc(CN2CCS(=O)(=O)CC2)c1 ; O=C(Nc1ccc(-c2nc(-c3ccc(C(F)(F)F)s3)n[nH]2)cc1)c1cccc(CN2CCS(=O)(=O)CC2)c1 ; O=C(Nc1ccc(-c2nc(Cc3ccccc3)n[nH]2)cc1)c1cccc(CN2CCS(=O)(=O)CC2)c1 ; Cc1ccc(Cc2n[nH]c(-c3ccc(NC(=O)c4cccc(CN5CCS(=O)(=O)CC5)c4)cc3)n2)cc1]

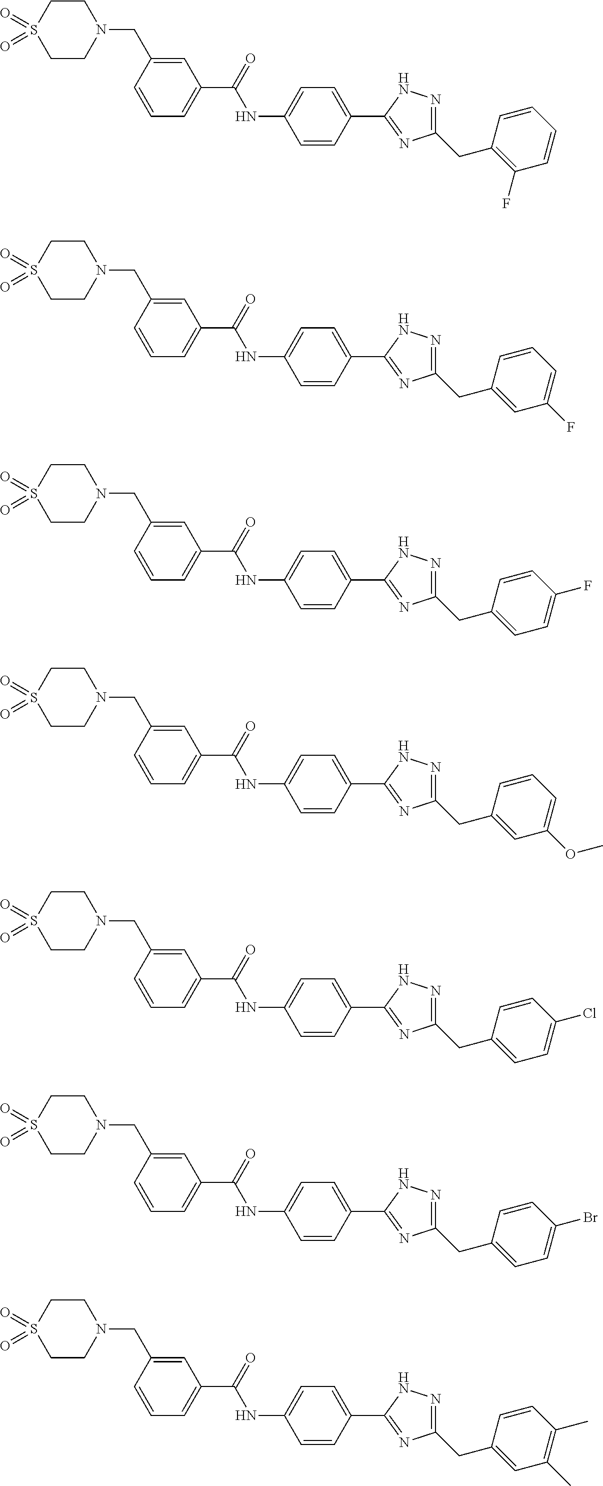

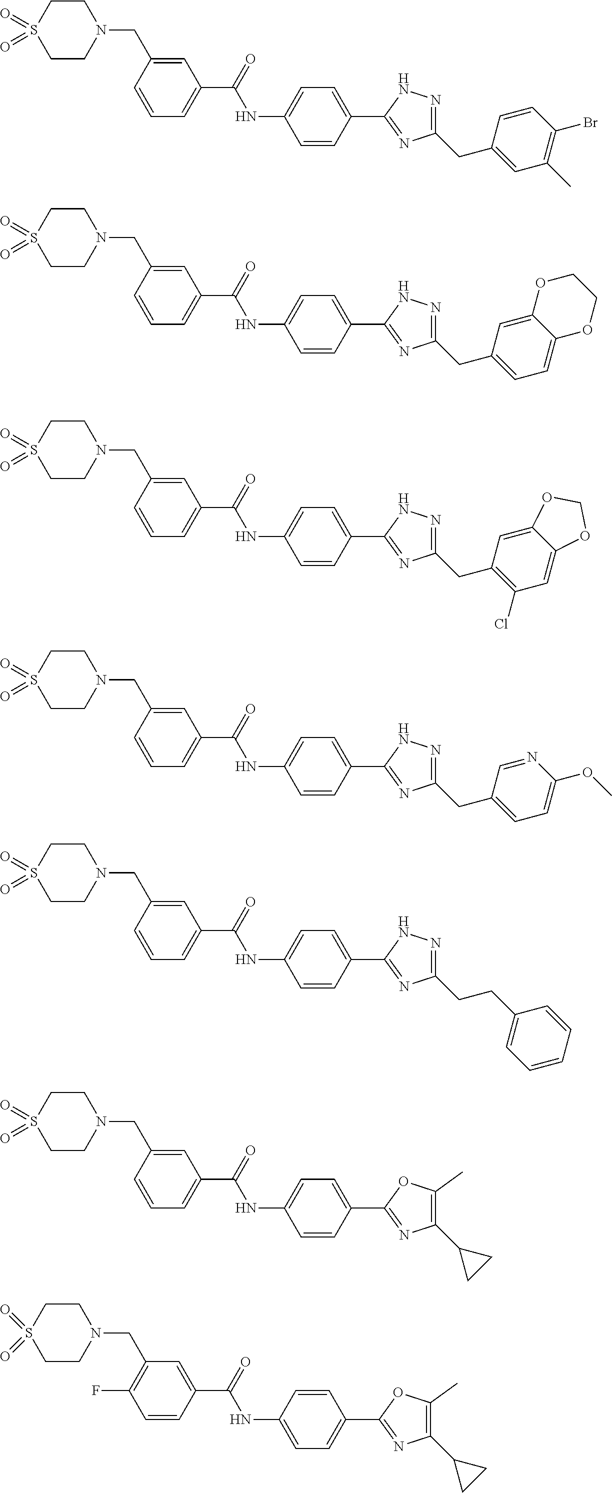

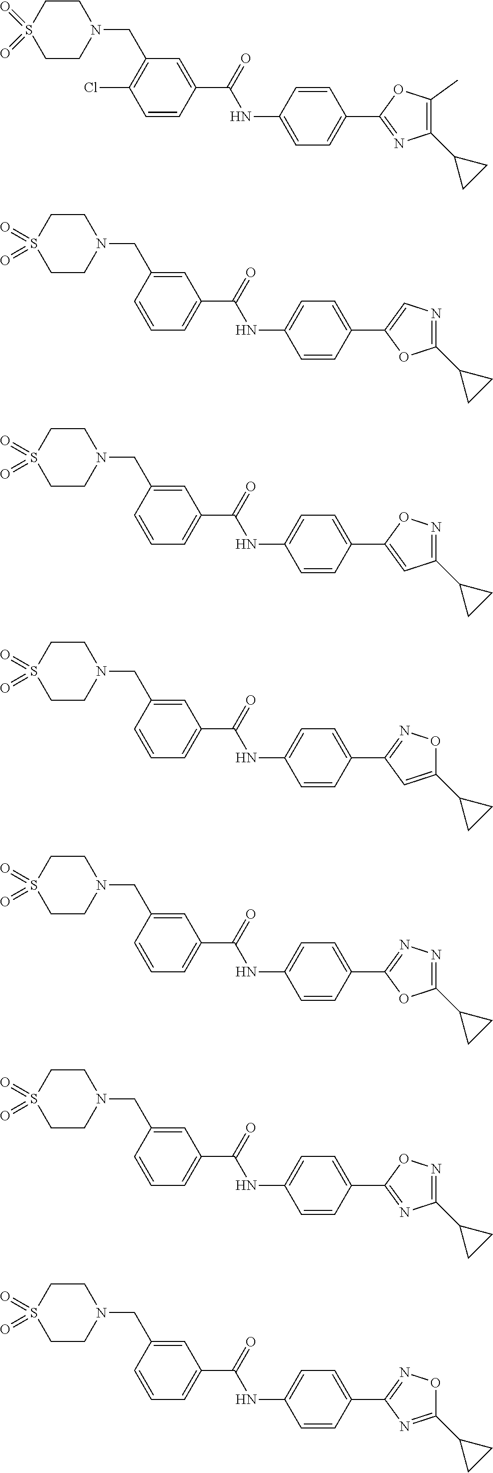

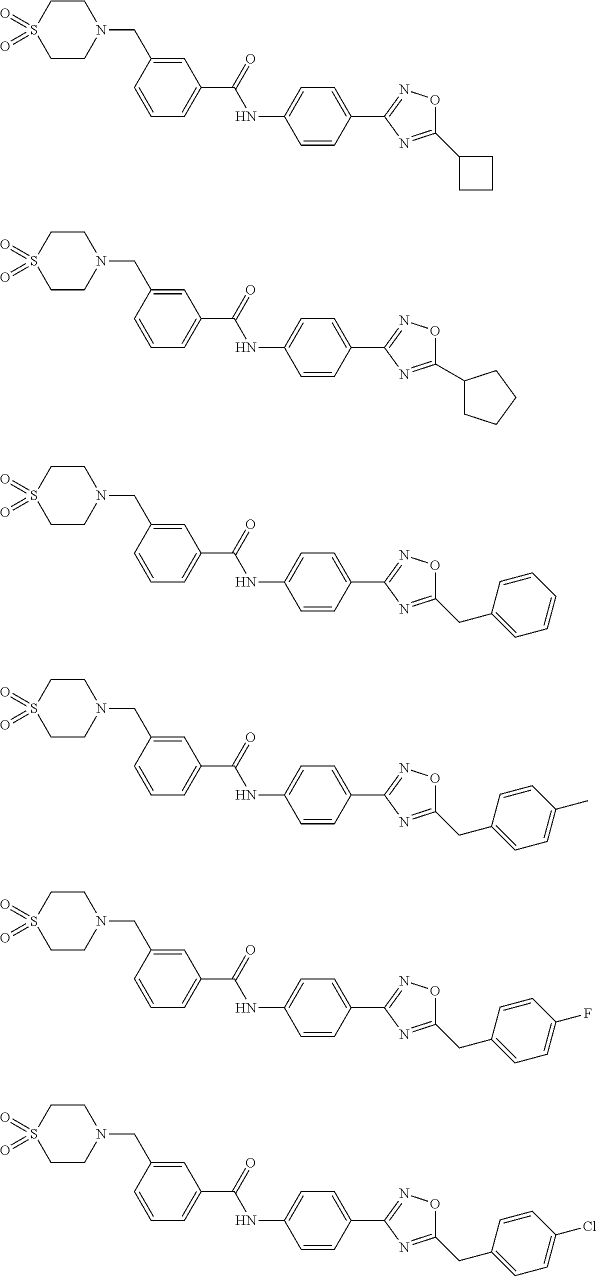 [SMILES: O=C(Nc1ccc(-c2noc(C3CCC3)n2)cc1)c1cccc(CN2CCS(=O)(=O)CC2)c1 ; O=C(Nc1ccc(-c2noc(C3CCCC3)n2)cc1)c1cccc(CN2CCS(=O)(=O)CC2)c1 ; O=C(Nc1ccc(-c2noc(Cc3ccccc3)n2)cc1)c1cccc(CN2CCS(=O)(=O)CC2)c1 ; Cc1ccc(Cc2nc(-c3ccc(NC(=O)c4cccc(CN5CCS(=O)(=O)CC5)c4)cc3)no2)cc1 ; O=C(Nc1ccc(-c2noc(Cc3ccc(F)cc3)n2)cc1)c1cccc(CN2CCS(=O)(=O)CC2)c1 ; O=C(Nc1ccc(-c2noc(Cc3ccc(Cl)cc3)n2)cc1)c1cccc(CN2CCS(=O)(=O)CC2)c1]

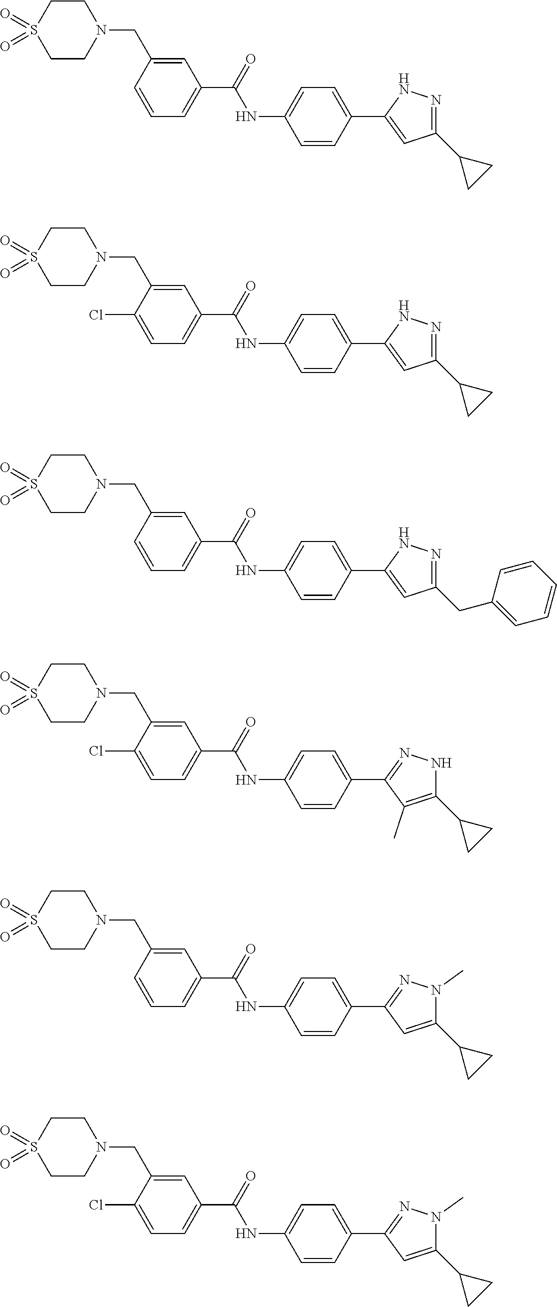 [SMILES: O=C(Nc1ccc(-c2cc(C3CC3)n[nH]2)cc1)c1cccc(CN2CCS(=O)(=O)CC2)c1 ; O=C(Nc1ccc(-c2cc(C3CC3)n[nH]2)cc1)c1ccc(Cl)c(CN2CCS(=O)(=O)CC2)c1 ; O=C(Nc1ccc(-c2cc(Cc3ccccc3)n[nH]2)cc1)c1cccc(CN2CCS(=O)(=O)CC2)c1 ; Cc1c(-c2ccc(NC(=O)c3ccc(Cl)c(CN4CCS(=O)(=O)CC4)c3)cc2)n[nH]c1C1CC1 ; Cn1nc(-c2ccc(NC(=O)c3cccc(CN4CCS(=O)(=O)CC4)c3)cc2)cc1C1CC1 ; Cn1nc(-c2ccc(NC(=O)c3ccc(Cl)c(CN4CCS(=O)(=O)CC4)c3)cc2)cc1C1CC1]

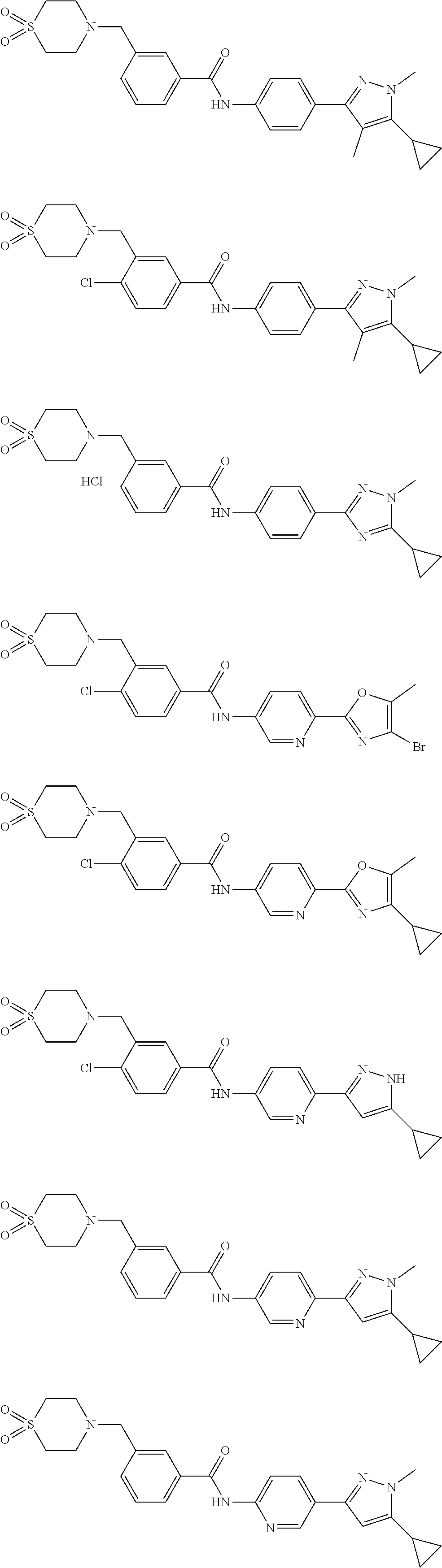

22. A pharmaceutical composition comprising a compound as defined in claim 1 and a pharmaceutically acceptable excipient.

23. The compound according to claim 1 for use in medicine.

24. A method of treating cancer, comprising administering a compound according to claim 1 to a patient in need thereof.

25. The method according to claim 24, wherein the cancer is colorectal cancer, pancreatic cancer, breast cancer, prostate cancer, esophageal cancer, gastric cancer, blood cancer, lung cancer, brain cancer, medulloblastic cancer, skin cancer, basal cell carcinoma, head and neck cancer, ovarian cancer, bladder cancer, kidney cancer, lung cancer, breast cancer, pancreatic cancer or stomach cancer.
